(19) United States
(12) Patent Application Publication
(10) Pub. No.: US 2025/0256007 A1
(43) Pub. Date: Aug. 14, 2025

(54) GELMA POLYMER COMPOSITIONS COMPRISING CELLS

(71) Applicant: GelMEDIX, Inc., Cambridge, MA (US)

(72) Inventors: Max COTLER, Littleton, MA (US); Noel VERA-GONZALEZ, Chelsea, MA (US); Arthur DRISCOLL, Reading, MA (US)

(21) Appl. No.: 18/857,683

(22) PCT Filed: Apr. 19, 2023
(86) PCT No.: PCT/US2023/019141

§ 371 Date: Oct. 17, 2024

Related U.S. Application Data
(60) Provisional application No. 63/332,963, filed on Apr. 20, 2022.
(60) Provisional application No. 63/410,041, filed on Sep. 26, 2022.

(51) Int. Cl. A61L 27/22; A61L 27/16; A61L 27/26; A61L 27/38; A61L 27/52; A61L 27/54; C08L 33/08

(52) U.S. Cl. CPC A61L 27/222; A61L 27/16; A61L 27/26; A61L 27/3808; A61L 27/3813; A61L 27/3834; A61L 27/52; A61L 27/54; C08L 33/08; A61L 2400/06; A61L 2430/16

(57) ABSTRACT

The present disclosure provides improved polymer compositions, such as GelMA polymer compositions. In certain embodiments, the improved polymer compositions can be used for delivering one or more therapeutic agents, such as cells, to a target therapeutic area, such as the eye of a subject. In certain embodiments, the improved polymer compositions are hydrogels which comprises gelatin methacryloyl (i.e., GelMA) or polymerically crosslinked derivatives thereof.

### RELATED APPLICATIONS

This application claims priority to U.S. Provisional Patent Application No. 63/332,963, filed Apr. 20, 2022, and U.S. Provisional Patent Application No. 63/410,041, filed Sep. 26, 2022. The entire contents of these applications are incorporated herein by reference.

### FIELD OF THE DISCLOSURE

The present disclosure provides improved polymer compositions, such as GelMA polymer compositions. In certain embodiments, the improved polymer compositions can be used for delivering one or more therapeutic agents, such as cells, to a target therapeutic area, such as the eye of a subject. In certain embodiments, the improved polymer compositions are hydrogels which comprises gelatin methacryloyl (i.e., GelMA) or polymerically crosslinked derivatives thereof.

### INTRODUCTION

GelMA polymer compositions have emerged as an effective material for use in sealing, repairing, and/or treating injuries, defects, or diseases in the soft tissues of subject. The design and production of improved GelMA polymer compositions for this purpose is an active field of study.

There remains a need for improved GelMA polymer compositions, methods for producing GelMA polymer compositions, and therapeutic applications for GelMA polymer compositions.

### SUMMARY

The details of various embodiments of the present disclosure are set forth in the description below.

In certain embodiments, the present disclosure provides polymer compositions which comprise at least one chemically modified gelatin. In certain embodiments, the polymer composition comprises at least one acrylated gelatin. In certain embodiments, the polymer composition comprises at least one methacrylated gelatin(GelMA). In certain embodiments, the polymer composition comprises between about 0.5% to about 5.0% w/v of chemically modified gelatin. In certain embodiments, the polymer composition comprises between about 0.5% to about 5.0% w/v of acrylated gelatin. In certain embodiments, the polymer composition comprises between about 0.5% to about 5.0% w/v of GelMA.

In certain embodiments, the polymer composition comprises at least one chemically modified gelatin (optionally an acrylated gelatin, such as GelMA) and at least one polymer crosslinking initiator (e.g., photoinitiator). In certain embodiments, the polymer composition comprises: (i) at least one chemically modified gelatin (optionally an acrylated gelatin); (ii) at least one polymer crosslinking initiator; and (iii) at least one therapeutic agent (e.g., a cell). In certain embodiments, the polymer composition comprises: (i) at least one chemically modified gelatin (optionally an methacrylated gelatin, such as GelMA); (ii) at least one polymer crosslinking initiator; and (iii) at least one cell In certain embodiments, the polymer composition is a precursor polymer composition. In certain embodiments, the polymer composition is a gel polymer composition.

In certain embodiments, the polymer composition comprises at least one crosslinking initiator. In certain embodiments, the crosslinking initiator comprises one or more light-activated photo-initiators, optionally one or more photo-initiators activated by visible light. In certain embodiments, the polymer crosslinking initiator comprises eosin Y, N-vinylcaprolactam, triethanolamine, or any combination thereof. In certain embodiments, the polymer crosslinking initiator comprises eosin Y disodium salt (EYDS), N-vinylcaprolactam (NVC), triethanolamine, or any combination thereof. In certain embodiments, the polymer crosslinking initiator comprises eosin Y disodium salt (EYDS), N-Vinylpyrrolidone (NVP), triethanolamine, or any combination thereof. In certain embodiments, the polymer crosslinking initiator comprises: (i) about 50 μM eosin Y or eosin Y disodium salt (EYDS); (ii) from about 3.5 to about 5.0 μL/mL of N-vinylcaprolactam (NVC) or N-Vinylpyrrolidone (NVP); and (iii) triethanolamine. In certain embodiments, the polymer crosslinking initiator comprises: (i) about 50 μM eosin Y disodium salt (EYDS); (ii) about 5.0 μL/mL N-Vinylpyrrolidone (NVP); and (iii) about 1.5% v/v of triethanolamine.

In certain embodiments, the chemically modified gelatin is an acrylated gelatin. In certain embodiments, the acrylated gelatin has a degree of acrylation from about 5-40%. In certain embodiments, the acrylated gelatin has a degree of acrylation from 5-20%. In certain embodiments, the acrylated gelatin has a degree of acrylation of about 5%, about 10%, or about 15%.

In certain embodiments, the chemically modified gelatin is methacrylated gelatin(GelMA). In certain embodiments, the GelMA has a degree of methacrylation from about 5-40%. In certain embodiments, the GelMA has a degree of methacrylation from 5-20%. In certain embodiments, the GelMA has a degree of methacrylation of about 5%, about 10%, or about 15%.

In certain embodiments, the polymer composition comprises from about 2% to about 5% w/v of the chemically modified gelatin (e.g., GelMA). In certain embodiments, the polymer composition comprises from about 3% to about 5% w/v of the chemically modified gelatin (e.g., GelMA). In certain embodiments, the polymer composition comprises from about 3% to about 4% w/v of the chemically modified gelatin (e.g., GelMA). In certain embodiments, the polymer composition includes about 3.5% w/v of the chemically modified gelatin (e.g., GelMA). In certain embodiments, the polymer composition comprises from about 3% to about 4% w/v of GelMA. In certain embodiments, the polymer composition includes about 3.5% w/v of GelMA.

In certain embodiments, the polymer composition comprises a combination of a first GelMA mixture and a second GelMA mixture. In certain embodiments, the polymer composition comprises a combination of a first GelMA mixture and a second GelMA mixture, wherein the total GelMA in the polymer composition is from about 0.5% to about 5% w/v. In certain embodiments, the total GelMA in the polymer composition is from about 2% to about 5% w/v. In certain embodiments, the total GelMA in the polymer composition is from about 2% to about 4% w/v. In certain embodiments, total GelMA in the polymer composition is from about 3% to about 5% w/v. In certain embodiments, the total GelMA in the polymer composition is from about 3% to about 4% w/v. In certain embodiments, the total GelMA in the polymer composition is from about 3.5% w/v.

In certain embodiments, the polymer composition comprises from about 0.5% to about 3% of the first GelMA mixture, and about 0.5% to about 3% of the second GelMA mixture. In certain embodiments, the polymer composition comprises from about 0.5% to about 1.5% of the first GelMA mixture, and about 2% to about 3% of the second GelMA mixture. In certain embodiments, the polymer composition comprises about 1% of the first GelMA mixture and about 2.5% of the second GelMA mixture.

In certain embodiments, the first GelMA mixture includes GelMA having a high average molecular weight and a low degree of methacrylation (DOM). In certain embodiments, the second GelMA mixture includes GelMA having a low average molecular weight and a high degree of methacrylation (DOM). In certain embodiments, the first GelMA mixture includes GelMA having a high average molecular weight and a low DOM; and the second GelMA mixture includes GelMA having a low average molecular weight and a high DOM. In certain embodiments, the first GelMA mixture includes GelMA having an average molecular weight from 140-180 kDa and a DOM from 5% to 40%; and the second GelMA mixture includes GelMA having an average molecular weight from 75-115 kDa and a DOM from 50% to 80%. In certain embodiments, the first GelMA mixture includes GelMA having an average molecular weight from 140-180 kDa and a DOM from 5% to 20%; and the second GelMA mixture includes GelMA having an average molecular weight from 80-100 kDa and a DOM from 50% to 70%. In certain embodiments, the first GelMA mixture includes GelMA having an average molecular weight of about 160 kDa and a DOM of about 10%; and the second GelMA mixture includes GelMA having an average molecular weight of about 90 kDa and a DOM of about 60%.

In certain embodiments, the polymer composition comprises: from about 0.5% to about 3% w/v of a first GelMA mixture, which includes GelMA having a high average molecular weight and a low DOM; and from about 0.5% to about 3% of a second GelMA mixture, which includes GelMA having a low average molecular weight and a high DOM. In certain embodiments, the polymer composition comprises: from about 0.5% to about 3% w/v of a first GelMA mixture which includes GelMA having an average molecular weight from 140-180 kDa and a DOM from 5% to 40%; and from about 0.5% to about 3% w/v of a second GelMA mixture which includes GelMA having an average molecular weight from 75-115 kDa and a DOM from 50% to 80%. In certain embodiments, the polymer composition comprises: from about 0.5% to about 1.5% w/v of a first GelMA mixture, which includes GelMA having an average molecular weight from 140-180 kDa and a DOM from 5% to 40%; and from about 2% to about 3% w/v of a second GelMA mixture which includes GelMA having an average molecular weight from 75-115 kDa and a DOM from 50% to 80%. In certain embodiments, the polymer composition comprises: from about 0.5% to about 1.5% w/v of a first GelMA mixture, which includes GelMA having an average molecular weight from 140-180 kDa and a DOM from 5% to 20%; and about 2% to about 3% w/v of a second GelMA mixture which includes GelMA having an average molecular weight from 85-100 kDa and a DOM from 50% to 70%.

In certain embodiments, the polymer composition comprises: about 1% w/v of a first GelMA mixture which includes GelMA having an average molecular weight of about 160 kDa and a DOM of about 10%; and about 2.5% w/v of a second GelMA mixture which includes GelMA having an average molecular weight of about 90 kDa and a DOM of about 60%.

In certain embodiments, at least one cell comprises an endothelial cell. In certain embodiments, at least one cell comprises a human umbilical vein endothelial cell (HUVEC). In certain embodiments, at least one cell comprises an epithelial cell. In certain embodiments, at least one cell comprises a human retinal pigment epithelium cell (HRPEC). In certain embodiments, at least one cell comprises a human retinal pigment epithelium cell (HRPEC) derived from induced pluripotent stem cells (iPSC). In certain embodiments, at least one cell comprises an ocular cell, or a pluripotent or embryonic stem cell derived ocular cell.

In certain embodiments, the polymer composition further comprises at least 0.1% (w/v) of a hydrophilic non-ionic surfactant. In certain embodiments, the hydrophilic non-ionic surfactant comprises at least one poloxamer surfactant such as Poloxamer 407. In certain embodiments, the composition comprises about 0.2% (w/v) of a poloxamer surfactant such as Poloxamer 407.

In certain embodiments, the present disclosure describes a precursor polymer composition, comprising a polymer composition of the present disclosure. In certain embodiments, the present disclosure describes a gel polymer composition formed by photocrosslinking a precursor polymer composition of the present disclosure. In certain embodiments, the present disclosure describes a hydrogel polymer composition formed by photocrosslinking a precursor polymer composition of the present disclosure.

In certain embodiments, the present disclosure provides a method for treating and/or repairing a defect, injury, and/or disease in a target soft tissue of a subject. In certain embodiments, the present disclosure provides a method for treating and/or repairing a defect, injury, and/or disease in a target soft tissue of a subject, said method comprising: providing a precursor polymer composition of the present disclosure; administering the precursor polymer composition onto or under a surface of the target soft tissue of the subject, optionally the location of the soft tissue defect, injury, and/or disease; and crosslinking the precursor polymer composition by exposing the polymer crosslinking initiator in the polymer composition to crosslinking conditions, wherein the crosslinking of the precursor polymer composition produces a gel polymer composition.

In certain embodiments, the present disclosure provides a method for treating a defect, injury, and/or disease in a target soft tissue of a subject, said method comprising: providing a gel polymer composition of the present disclosure; and administering the gel polymer composition onto, under, or near a surface of the target soft tissue of the subject. In certain embodiments, the gel polymer composition is administered at location of the soft tissue defect, injury, and/or disease.

In certain embodiments, the target soft tissue is ocular tissue. In certain embodiments, the target soft tissue is subconjunctival ocular tissue or retinal ocular tissue. In certain embodiments, the polymer composition is applied onto or under the surface of the ocular tissue by subconjunctival injection, subretinal injection, or suprachoroidal injection.

In certain embodiments, the defect, injury, and/or disease of the target soft tissue comprises an ocular defect, injury and/or disease; optionally an ocular ulcer such as a corneal ulcer from infections, injuries, perforations, or other defect. In certain embodiments, the ocular defect, injury and/or disease comprises a retinal degeneration disease. In certain embodiments, the ocular defect, injury and/or disease comprises age-related macular degeneration (AMD). In certain embodiments, the ocular defect, injury and/or disease comprises retinitis pigmentosa.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. TA provides an example of a reaction in which gelatin is modified with methacrylic anhydride (MA) to form a methacryloyl-substituted gelatin (GelMA). FIG. 1B provides an example of a reaction in which hyaluronic acid is modified with glycidyl methacrylate to form a methacrylated hyaluronic acid (MeHA). FIG. 1C provides an example of a reaction in which Poly(ethylene glycol) (PEG) is modified with acryloyl chloride to form Poly(ethylene glycol) diacrylate (PEGDA).

FIG. 2 provides a method 100 for producing gel polymer compositions of the present disclosure.

FIG. 3 provides an example of a series of reactions to produce a GelMA hydrogel polymer composition from gelatin methacryloyl polymer precursors using a photoinitiator element and light energy.

FIG. 4A and FIG. 4B present the results of a study on the correlation between the degree of crosslinking within hydrogels of the present disclosure as a function of photopolymerization time. FIG. 4A shows degree (%) of crosslinking for HAMA—only hydrogels; FIG. 4B shows the ratio of [ME methyl groups to lysine CH₂ groups] for GelMA-only hydrogels.

FIG. 5A, FIG. 58, FIG. 5C, and FIG. 5D present the results of a study on the swelling ratios of hydrogels of the present disclosure having various GelMA, HAMA, and PEGDA concentrations. FIG. 5A and FIG. 5B show Swelling Ratio measurements for four hydrogel formulations of the present disclosure; FIG. 5C shows Swelling Ratio measurements for four hydrogel formulations of the present disclosure under re-swelling conditions; FIG. 5D shows Swelling Ratio measurements for seven GelMA, PEGDA, and GelMA+PEGDA hydrogel formulations of the present disclosure.

FIG. 6A and FIG. 6B present the results of a study on the swelling ratios of hydrogels of the present disclosure prepared with an active agent and having various GelMA, HAMA, and PEGDA concentrations. FIG. 6A shows Swelling Ratio measurements for six hydrogel formulations of the present disclosure, both with and without an active agent; FIG. 6B shows Swelling Ratio measurements for six hydrogel formulations of the present disclosure, both with and without an active agent, under re-swelling conditions.

FIG. 7A, FIG. 7B, FIG. 7C, and FIG. 7D present the results of a study on the drug release profiles of hydrogels of the present disclosure having various GelMA, HAMA, and PEGDA concentrations. FIG. 7A shows drug release profiles for G4-H_{M}1-P1 and G4-H_{G}3-P1 hydrogels formulations of the present disclosure, up to 10-13 days; FIG. 7B and FIG. 7C show extended drug release profiles for G4-H_{M}1-P1 up to 35 days (FIG. 7B) and 65 days (FIG. 7C); FIG. 7D shows drug release profiles for G4-H_{M}1-P1, G4-P1 and G7-P1 hydrogels formulations of the present disclosure.

FIG. 8A and FIG. 8B present the results of a study on the effect of vacuum drying on the drug release profile of hydrogels of the present disclosure prepared with an active agent and having various GelMA, HAMA, and PEGDA concentrations. FIG. 8A shows drug release profiles for G4-H_{M}1-P1 hydrogel formulations of the present disclosure, both in “wet” and “vacuum-dried” forms; FIG. 8B shows drug release profiles for G7-P1 and G4-P1 hydrogel formulations of the present disclosure, both in “wet” and “vacuum-dried” forms.

FIG. 9A and FIG. 9B present the results of a study on the effect of hydrogel shape and hydration status on the drug release profile of hydrogels of the present disclosure prepared with an active agent and having various GelMA, HAMA, and PEGDA concentrations. FIG. 9A shows the Total Drug Release profiles for G4-H_{M}1-P1 hydrogel formulations of the present disclosure, both in “rod” and “disk” forms (including wet, vacuum dried, and freeze-dried rod forms); FIG. 9B shows the Percentage Drug Release profiles for G4-H_{M}1-P1 hydrogel formulations of the present disclosure, both in “rod” and “disk” forms (including wet, vacuum dried, and freeze-dried rod forms).

FIG. 10 provides the results of a study on the correlation between the release profile of a GelMA+PEGDA hydrogel of the present disclosure and the degree of GelMA methacrylation within the hydrogels.

FIG. 11A and FIG. 11B present the results of a cellular aggregation and viability study using certain embodiments of hydrogel of the present disclosure. FIG. 11A provides Calcein AM+ based imaging for certain hydrogel samples; FIG. 11B shows Calcein AM+ percentage (Live/Live+Dead) quantification data for certain hydrogel samples.

FIG. 12A, FIG. 12B, FIG. 12C, FIG. 12D, FIG. 12E, FIG. 12F, FIG. 12G, FIG. 12H, and FIG. 12I present GFP+ cell imaging results for a transwell underside cell growth study using Human Umbilical Vein Endothelial Cells (HUVECs) and certain embodiments of hydrogel of the present disclosure.

FIG. 13A, FIG. 133, FIG. 13C, FIG. 13D, and FIG. 13E present confocal imaging results for a transwell underside cell growth study using Human Umbilical Vein Endothelial Cells (HUVECs) and certain embodiments of hydrogel of the present disclosure.

FIG. 14A, FIG. 14B, FIG. 14C, FIG. 14D, FIG. 14E, FIG. 14F, FIG. 14G, FIG. 14H, FIG. 14I, and FIG. 14J present GFP+ cell imaging results for a transwell underside cell growth study using Human Umbilical Vein Endothelial Cells (HUVECs) and certain embodiments of hydrogel of the present disclosure.

FIG. 15A, FIG. 153, FIG. 15C, FIG. 15D, FIG. 15E, FIG. 15F, and FIG. 15G present Calcein AM+ based imaging results for a transwell underside cell growth study using Human Retinal Pigment Epithelium Cells (HRPEC) and certain embodiments of hydrogel of the present disclosure.

FIG. 16A, FIG. 16B, and FIG. 16C present Calcein AM+ based imaging results for a transwell topside cell growth study using Human Retinal Pigment Epithelium Cells (HRPEC) and certain embodiments of hydrogel of the present disclosure.

FIG. 17A provides live-cell Calcein AM imaging results related to in vitro retinal cell growth studies for hydrogel cell delivery formulations (˜20 million cells/mL) of the present disclosure. FIG. 17B provides measurement for fluid shear rates related to the extrusion of GelMA precursor polymer formulations through the small-diameter needle. FIG. 17C provides live-cell Calcein AM imaging results related to in vitro retinal cell growth studies for hydrogel cell delivery formulations (˜1 million cells/mL) of the present disclosure. FIG. 17D provides live-cell imaging results (anti-CD73 and anti-rhodopsin) related to in vitro retinal cell growth studies for hydrogel cell delivery formulations of the present disclosure.

FIG. 18A provides study results for cell localization and migration studies for hydrogel formulations of the present disclosure. FIG. 18B provides study results for hydrogel degradation studies for hydrogel formulations of the present disclosure. FIG. 18C and FIG. 18D show images of retinal detachment related to hydrogel formulations of the present disclosure. FIG. 18E is an image of human RPE cell deposition onto native pig RPE layers.

FIG. 19 provides study results for GelMA propagator studies for hydrogel formulations of the present disclosure.

FIG. 20 provides normalized, cell-viability study results related to hydrogel formulations of the present disclosure.

FIG. 21 provides cell growth study results related to hydrogel formulations of the present disclosure.

### DETAILED DESCRIPTION

### I. Polymer Compositions

### General

The present disclosure provides polymer compositions (e.g., GelMA polymer compositions) which have one or more advantages over compositions in current commercial use or known in the art. In certain embodiments, the polymer compositions have one or more of the following advantages relative to one or more composition in current commercial use or known in the art: (i) lower in cost; (ii) easier to produce; (iii) improved biocompatibility; (iv) faster and/or stronger crosslinking and stabilization; (v) easier and/or more stable application; (vi) stronger adhesion and/or retention to target surface; (vii) degradation characteristics which can be engineered and adjusted; (viii) a smooth surface once applied; and/or (ix) higher cell viability or improved delivery for an encapsulated cell. In certain embodiments, polymer compositions of the present disclosure permit controlled and sustained release of one or more therapeutic agents or cells over a period of time. As such, the polymer compositions of the present disclosure present clear improvements over compositions in current commercial use and currently known in the art.

The term “polymer composition” as used herein can refer to a precursor polymer composition (e.g., a polymer composition before crosslinking polymerization) and/or a gel polymer composition (e.g., a polymer composition after crosslinking polymerization), as provided by the corresponding context of the disclosure. Examples of gel polymer compositions include hydrogels, and polymer compositions with increased viscosity (e.g., soft gels) resulting from crosslinking polymerization in the polymer composition.

In general, reference to a polymer component in the present disclosure (e.g., GelMA, MeHA, PEGDA) can refer to a polymer precursor component (e.g., monomer or precursor oligomer), a crosslinked form of the polymer component in an oligomer (e.g., crosslinked oligomer), and/or a polymerized form of the polymer component in a gel polymer composition (e.g., hydrogel polymer), according to the context within the present disclosure.

In certain embodiments, the polymer compositions comprises a chemically-modified gelatin, such as gelatin methacryloyl (i.e., GelMA). In certain embodiments, the polymer compositions comprises chemically-modified gelatin (e.g., GelMA) and one or more crosslinking agents. In certain embodiments, the polymer compositions comprises chemically-modified gelatin (e.g., GelMA) and one or more polymer crosslinking initiators, such as light-activated photo-initiator elements. In certain embodiments, the polymer compositions comprises chemically-modified gelatin (e.g., GelMA), one or more crosslinking agents, and one or more polymer crosslinking initiators, such as light-activated photo-initiator elements.

In certain embodiments, the polymer compositions comprises chemically-modified gelatin (e.g., GelMA) and chemically modified hyaluronic acid (e.g., MeHA). In certain embodiments, the polymer compositions comprises chemically-modified gelatin (e.g., GelMA), chemically modified hyaluronic acid (e.g., MeHA), and one or more crosslinking agents. In certain embodiments, the polymer compositions comprises chemically-modified gelatin (e.g., GelMA), chemically modified hyaluronic acid (e.g., MeHA), and one or more polymer crosslinking initiators, such as light-activated photo-initiator elements. In certain embodiments, the polymer compositions comprises chemically-modified gelatin (e.g., GelMA), chemically modified hyaluronic acid (e.g., MeHA), one or more crosslinking agents, and one or more polymer crosslinking initiators, such as light-activated photo-initiator elements. In certain embodiments, the polymer composition comprises an unmodified HA. In certain embodiments, the polymer composition comprises an unmodified HA and a chemically modified HA (e.g., MeHA).

In certain embodiments, the polymer compositions comprises chemically-modified gelatin (e.g., GelMA) and chemically modified Poly(ethylene glycol) (PEG) (e.g., PEGDA). In certain embodiments, the polymer compositions comprises chemically-modified gelatin (e.g., GelMA), chemically modified PEG (e.g., PEGDA), and one or more crosslinking agents. In certain embodiments, the polymer compositions comprises chemically-modified gelatin (e.g., GelMA), chemically modified PEG (e.g., PEGDA), and one or more polymer crosslinking initiators, such as light-activated photo-initiator elements. In certain embodiments, the polymer compositions comprises chemically-modified gelatin (e.g., GelMA), chemically modified PEG (e.g., PEGDA), one or more crosslinking agents, and one or more polymer crosslinking initiators, such as light-activated photo-initiator elements. In certain embodiments, the polymer composition comprises an unmodified PEG. In certain embodiments, the polymer composition comprises an unmodified PEG and a chemically modified PEG (e.g., PEGDA).

In certain embodiments, the polymer compositions comprises chemically-modified gelatin (e.g., GelMA), chemically modified hyaluronic acid (e.g., MeHA), and chemically modified PEG (e.g., PEGDA). In certain embodiments, the polymer compositions comprises chemically-modified gelatin (e.g., GelMA), chemically modified hyaluronic acid (e.g., MeHA), chemically modified PEG (e.g., PEGDA), and one or more crosslinking agents. In certain embodiments, the polymer compositions comprises chemically-modified gelatin (e.g., GelMA), chemically modified hyaluronic acid (e.g., MeHA), chemically modified PEG (e.g., PEGDA), and one or more polymer crosslinking initiators, such as light-activated photo-initiator elements. In certain embodiments, the polymer compositions comprises chemically-modified gelatin (e.g., GelMA), chemically modified hyaluronic acid (e.g., MeHA), chemically modified PEG (e.g., PEGDA), one or more crosslinking agents, and one or more polymer crosslinking initiators, such as light-activated photo-initiator elements. In certain embodiments, the polymer composition comprises an unmodified HA and/or an unmodified PEG.

In certain embodiments, the polymer compositions do not comprise a hydrolyzing enzyme. In certain embodiments, the polymer compositions do not comprise a glycosidase hydrolyzing enzyme.

In certain embodiments, the gel polymer composition is a hydrogel. A hydrogel generally comprises a crosslinked polymeric framework which encompasses a network of pores filled with an interstitial solvent (e.g., a fluid) which includes water. In certain embodiments, a hydrogel polymer composition has a water content of about 80% or more. In certain embodiments, a hydrogel polymer composition has a water content of more than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or more than about 99%.

In certain embodiments, a polymer composition (e.g., hydrogel or hydrogel precursor) of the present disclosure (e.g., GelMA polymer composition) is a primary polymer composition, and can include or be combined with one of more secondary hydrogel-forming polymers components (i.e., polymers or precursors thereof). In certain embodiments, a polymer composition (e.g., hydrogel or hydrogel precursor) of the present disclosure (e.g., GelMA polymer composition) is a primary polymer composition, and can include or be combined with one of more secondary hydrogel-forming polymers components selected from acrylamide, acrylic acid, alginate, alginate methacrylate, cellulose, chitosan, chitosan methacrylate, dimethacrylamide, methylenebisacrylamide, fibronectin, gelatin, gelatin methacrylate, glycol chitosan, glycol chitosan methacrylate, hexyl methacrylate, hyaluronic acid, hyaluronic acid methacrylate, hydroxyethyl methacrylate, hydroxyethyl acrylate, isopropyl acrylamide, isopropyl methacrylamide, laminin, methacrylamide, methacrylic acid, polyamide, polycaprolactone, polyethylene-glycol (PEG), polyethylene-terephthalate, polylactic acid, polyurethane, polyvinyl alcohol, polyethyleneoxide dimethacrylate, siloxanes, polysiloxanes, or oligomer, polymers, and/or combinations thereof. In certain embodiments, a secondary hydrogel polymer (formed from the secondary hydrogel-forming polymer precursors) is covalently crosslinked with the primary gel polymer composition (e.g., GelMA polymer composition). In certain embodiments, the secondary hydrogel polymer (formed from the secondary hydrogel-forming polymer precursors) is not covalently crosslinked with the primary gel polymer composition (e.g., GelMA polymer composition), such, for example, the secondary hydrogel polymer forming a polymer network that is interwoven with the polymer network of the primary gel polymer composition.

In certain embodiments, a polymer composition of the present disclosure comprises one or more biocompatible polymer components or polysaccharides. In certain embodiments, a polymer composition of the present disclosure comprises one or more biocompatible polymer components or polysaccharides selected from agarose, alginates, amylopectin, amylose, carrageenan, cellulose, chitin, chitosans, chondroitin sulfate, collagen, dermatan sulfate, dextran, elastin, elastin-like polypeptides (ELPs), tropoelastin, fibrin, fibrinogen, fibronectin, gelatin, glycogen, heparan, heparan sulfate, heparin, heparin sulfate, hyaluronans, hyaluronic acid, keratan sulfate, laminin, pectin, polyglycerol sebacate (PGS), polyethylene glycol (PEG), polylactic acid (PLA), polylysine, starch, thrombin, derivatives thereof, or a combination thereof.

In certain embodiments, a polymer composition of the present disclosure comprises one or more cell-adhesion agents selected from fibronectin, laminin, vitronectin, RGD, vixapatin, derivatives thereof, or a combination thereof.

In certain embodiments, a polymer composition of the present disclosure comprises one or more synthetic polymer components, such as a biocompatible synthetic polymer component. In certain embodiments, a polymer composition comprises one or more synthetic polymer components selected from polyurethanes, polysiloxanes, silicones, polyethylenes, polyvinyl pyrrolidones, polyhydroxy ethylmethacrylates (poly-HEMA), polymethyl methacrylates, polyvinyl alcohols, polyacrylic acids, polyacrylamides, polyethylene-co-vinyl acetates, polyethylene glycols, polymethacrylic acids, polylactic acids, polyglycolic acids, polylactide-co-glycolides, nylons, polyamides, polyanhydrides, polyethylene-co-vinyl alcohols, polycaprolactones, polyvinyl acetates, polyvinylhydroxides, polyethylene oxides, polyorthoesters, polyallyl amines, polyethylene imines, polylysines, polyarginines, derivatives thereof, or combinations and/or copolymers thereof.

In certain embodiments, a polymer composition of the present disclosure comprises one or more polymer components (e.g., monomers, precursors, polymers) which include a crosslinkable group. In certain embodiments, a polymer composition of the present disclosure comprises one or more polymer components which include a crosslinkable group selected from (or formed from reaction with) anhydrides, acid halides, carboxylic acids, diols, acrylic anhydrides, methacrylic anhydrides, acryloyl chlorides, acryloyl bromides, methacryloyl chlorides, methacryloyl bromides, acrylic acids, glycidyl methacrylates, methacrylic acids, dopamines, derivatives thereof, or combinations thereof.

In certain embodiments, a hydroxy ethylmethacrylate (HEMA) or polymer thereof can be present in a polymer composition at a concentration from about 1% and about 60% weight per volume (w/v).

In certain embodiments, a polymer composition of the present disclosure comprises one or more stabilizers and/or enhancers. In certain embodiments, a polymer composition of the present disclosure comprises one or more stabilizers and/or enhancers selected from polar amino acids (e.g., tyrosine, cysteine, serine, threonine, asparagine, glutamine, aspartic acid, glutamic acid, arginine, lysine, and histidine), amino acid analogues, amino acid derivatives, collagen, divalent cation chelators (e.g., ethylenediaminetetraacetic acid (EDTA) or salts thereof), or a combination thereof.

In certain embodiments, a polymer composition of the present disclosure can be clear and/or translucent. In certain embodiments, a polymer composition can be partially translucent or partially opaque. In certain embodiments, a polymer composition can be opaque.

In certain embodiments, the polymer compositions of the present disclosure can include the polymeric or therapeutic components, can be produced, can be analyzed or can be used as disclosed in US 20140377326, US 20150274805, US 20160175488, US 20170232138, US 20190022280 A1, WO 2020051133, and WO 2020081673, each of which is incorporated herein by reference in its entirety, insofar as each describes the composition, production, analysis and use of acrylated gelatin polymeric compositions such as GelMA hydrogels.

### Formulations

In certain embodiments, polymer compositions of the present disclosure comprises chemically-modified gelatin (e.g., GelMA), chemically modified hyaluronic acid (e.g., MeHA), chemically modified PEG (e.g., PEGDA); or any combination thereof. In certain embodiments, the polymer composition comprises chemically-modified gelatin (e.g., GelMA). In certain embodiments, the polymer composition comprises chemically modified hyaluronic acid (e.g., MeHA). In certain embodiments, the polymer composition comprises chemically modified PEG (e.g., PEGDA). In certain embodiments, the polymer composition comprises chemically-modified gelatin (e.g., GelMA) and chemically modified hyaluronic acid (e.g., MeHA). In certain embodiments, the polymer composition comprises chemically-modified gelatin (e.g., GelMA) and chemically modified PEG (e.g., PEGDA). In certain embodiments, the polymer composition comprises chemically modified hyaluronic acid (e.g., MeHA) and chemically modified PEG (e.g., PEGDA). In certain embodiments, the polymer composition comprises chemically-modified gelatin (e.g., GelMA), chemically modified hyaluronic acid (e.g., MeHA), and chemically modified PEG (e.g., PEGDA).

In certain embodiments, polymer compositions of the present disclosure comprises combinations of precursor polymer components according from Table 1 (percentages are w/v concentration in the total precursor polymer formulation). Unless stated otherwise, GelMA materials in Table 1 are 160/80 (i.e., have 160 kDa molecular weight (MW) and 80% degree of methacrylation (DoM)). Unless stated otherwise, HAMA materials in Table 1 are 500/30 (i.e., have 500 kDa molecular weight (MW) and 30% degree of methacrylation (DoM)). Unless stated otherwise, PEGDA materials in Table 1 are formed from 35 kDa PEG materials. Poloxamer 407=Px 407.

TABLE 1
Examples of Precursor Polymer Compositions

| Formulation | Modified Gelatin | Modified HA | Modified PEG | Other |
|---|---|---|---|---|
| G10-H_{M}1.5-P0.5(2K) | 10% GelMA | 1.5% HAMA | 0.5% PEGDA | — |
| | | | (2 kDa) | |
| G4-H_{G}3-P1 | 4% GelMA | 3% HAGM | 1% PEGDA | — |
| G4-H_{M}1-P1 | 4% GelMA | 1% HAMA | 1% PEGDA | — |
| G4-H_{M} 1-P0.67 | 4% GelMA | 1% HAMA | 0.67% PEGDA | — |
| G7-H_{G}3 | 7% GelMA | 3% HAGM | — | — |
| G7-H_{M}1 | 7% GelMA | 1% HAMA | — | — |
| G4-H_{G}3 | 4% GelMA | 3% HAGM | — | — |
| G4-H_{M}1 | 4% GelMA | 1% HAMA | — | — |
| G7-P1 | 7% GelMA | — | 1% PEGDA | — |
| G7(160/40)-P1 | 7% GelMA | — | 1% PEGDA | — |
| | (160/40) | | | |
| G5-P1 | 5% GelMA | — | 1% PEGDA | — |
| G5(160/40)-P1 | 5% GelMA | — | 1% PEGDA | — |
| | (160/40) | | | |
| G4-P1 | 4% GelMA | — | 1% PEGDA | — |
| G4-P1(2K) | 4% GelMA | — | 1% PEGDA | — |
| | | | (2 kDa) | |
| G4(160/40)-P1 | 4% GelMA | — | 1% PEGDA | — |
| | (160/40) | | | |
| G4(160/40)-P0.1 | 4% GelMA | — | 0.1% PEGDA | — |
| | (160/40) | | | |
| G20 | 20% GelMA | — | — | — |
| G20(160/40) | 20% GelMA | — | — | — |
| | (160/40) | | | |
| G15 | 15% GelMA | — | — | — |
| G15(160/40) | 15% GelMA | — | — | — |
| | (160/40) | | | |
| G10 | 10% GelMA | — | — | — |
| G10(160/40) | 10% GelMA | — | — | — |
| | (160/40) | | | |
| G7 | 7% GelMA | — | — | — |
| G7(160/40) | 7% GelMA | — | — | — |
| | (160/40) | | | |
| G5 | 5% GelMA | — | — | — |
| G5(160/10) | 5% GelMA | | | |
| | (160/10) | | | |
| G5(160/40) | 5% GclMA | — | — | — |
| | (160/40) | | | |
| G4 | 4% GelMA | — | — | — |
| G4(160/40) | 4% GelMA | — | — | — |
| | (160/40) | | | |
| G4(160/10) | 4% GelMA | | | |
| | (160/10) | | | |
| G1(160/10)G2.5(90/60) | 1% Gelma | | | |
| | (160/10) + | | | |
| | 2.5% Gelma | | | |
| | (90/60) | | | |
| G1(160/10)G2.5(90/60)P0.5 | 1% Gelma | | 0.5% PEGDA | |
| | (160/10) + | | (2 kDa) | |
| | 2.5% Gelma | | | |
| | (90/60) | | | |
| GM2(160/45)G2.5(90/45) | 1% Gelma | | | |
| | [glycidyl | | | |
| | methacrylate- | | | |
| | functionalized] | | | |
| | (160/45) + | | | |
| | 2.5% Gelma | | | |
| | (90/45) [glycidyl | | | |
| | methacrylate- | | | |
| | functionalized] | | | |
| H_{G}3-P1 | — | 3% HAGM | 1% PEGDA | — |
| H_{M}1-P1 | — | 1% HAMA | 1% PEGDA | — |
| H_{M}1-P0.67 | — | 1% HAMA | 0.67% PEGDA | — |
| H_{G}3 | — | 3% HAGM | — | — |
| H_{M}1 | — | 1% HAMA | — | — |
| P20 | — | — | 20% PEGDA | — |
| P10 | — | — | 10% PEGDA | — |
| P8 | — | — | 8% PEGDA | — |
| P6 | — | — | 6% PEGDA | — |
| P5 | — | — | 5% PEGDA | — |
| P4 | — | — | 4% PEGDA | — |

In certain embodiments, the polymer composition comprises about 4-20% w/v of chemically-modified gelatin (e.g., GelMA); about 0-1.5% w/v of chemically modified hyaluronic acid (e.g., MeHA); and about 0-5% w/v of chemically modified PEG (e.g., PEGDA). In certain embodiments, the polymer composition comprises about 4-10% w/v of chemically-modified gelatin (e.g., GelMA); about 1-1.5% w/v of chemically modified hyaluronic acid (e.g., MeHA); and about 0.1-5% w/v of chemically modified PEG (e.g., PEGDA).

In certain embodiments, the polymer composition comprises GelMA having about 160 kDa molecular weight (MW). In certain embodiments, the polymer composition comprises GelMA having about 160 kDa molecular weight (MW). In certain embodiments, the polymer composition comprises GelMA having from about 80% to about 90%0 degree of methacrylation (DoM). In certain embodiments, the polymer composition comprises GelMA having about 85% degree of methacrylation (DoM). In certain embodiments, the polymer composition comprises GelMA having about 80% degree of methacrylation (DoM). In certain embodiments, the polymer composition comprises GelMA having from about 75% to about 85% degree of methacrylation (DoM). In certain embodiments, the polymer composition comprises GelMA having from about 70% to about 80% degree of methacrylation (DoM). In certain embodiments, the polymer composition comprises GelMA having about 75% degree of methacrylation (DoM). In certain embodiments, the polymer composition comprises GelMA having about 70% degree of methacrylation (DoM). In certain embodiments, the polymer composition comprises GelMA having from about 65% to about 75% degree of methacrylation (DoM). In certain embodiments, the polymer composition comprises GelMA having from about 60% to about 70% degree of methacrylation (DoM). In certain embodiments, the polymer composition comprises GelMA having about 65% degree of methacrylation (DoM). In certain embodiments, the polymer composition comprises GelMA having about 60% degree of methacrylation (DoM). In certain embodiments, the polymer composition comprises GelMA having from about 55% to about 65% degree of methacrylation (DoM). In certain embodiments, the polymer composition comprises GelMA having from about 50% to about 60% degree of methacrylation (DoM). In certain embodiments, the polymer composition comprises GelMA having about 55% degree of methacrylation (DoM). In certain embodiments, the polymer composition comprises GelMA having about 50% degree of methacrylation (DoM). In certain embodiments, the polymer composition comprises GelMA having from about 45% to about 55% degree of methacrylation (DoM). In certain embodiments, the polymer composition comprises GelMA having from about 40% to about 50% degree of methacrylation (DoM). In certain embodiments, the polymer composition comprises GelMA having about 45% degree of methacrylation (DoM). In certain embodiments, the polymer composition comprises GelMA having about 40% DoM. In certain embodiments, the polymer composition comprises GelMA having from about 35% to about 45% degree of methacrylation (DoM). In certain embodiments, the polymer composition comprises GelMA having from about 30% to about 40% degree of methacrylation (DoM). In certain embodiments, the polymer composition comprises GelMA having about 35% degree of methacrylation (DoM). In certain embodiments, the polymer composition comprises GelMA having about 30% degree of methacrylation (DoM). In certain embodiments, the polymer composition comprises GelMA having from about 25% to about 35% degree of methacrylation (DoM). In certain embodiments, the polymer composition comprises GelMA having from about 20% to about 30% degree of methacrylation (DoM). In certain embodiments, the polymer composition comprises GelMA having about 25% degree of methacrylation (DoM). In certain embodiments, the polymer composition comprises GelMA having about 20% DoM. In certain embodiments, the polymer composition comprises GelMA having from about 15% to about 25% degree of methacrylation (DoM). In certain embodiments, the polymer composition comprises GelMA having from about 10% to about 20% degree of methacrylation (DoM). In certain embodiments, the polymer composition comprises GelMA having about 15% DoM. In certain embodiments, the polymer composition comprises GelMA having about 10% DoM. In certain embodiments, the polymer composition comprises GelMA having about 5% DoM. In certain embodiments, the polymer composition comprises GelMA having about 10-40% DoM. In certain embodiments, the polymer composition comprises GelMA having about 50-80% DoM. In certain embodiments, the polymer composition comprises GelMA having about 10-20% DoM. In certain embodiments, the polymer composition comprises GelMA having about 7% DoM. In certain embodiments, the polymer composition comprises GelMA having about 5% DoM. In certain embodiments, the polymer composition comprises GelMA having about 5-40% DoM. In certain embodiments, the polymer composition comprises GelMA having about 5-20% DoM. In certain embodiments, the polymer composition comprises GelMA having about 1-10% DoM.

In certain embodiments, the polymer composition comprises a combination of a first GelMA mixture and a second GelMA mixture. In certain embodiments, the polymer composition comprises a combination of a first GelMA mixture and a second GelMA mixture, wherein the total GelMA in the polymer composition is from about 0.5% to about 5%. In certain embodiments, the polymer composition comprises a combination of a first GelMA mixture and a second GelMA mixture, wherein the total GelMA in the polymer composition is from about 2% to about 5%. In certain embodiments, the polymer composition comprises a combination of a first GelMA mixture and a second GelMA mixture, wherein the total GelMA in the polymer composition is from about 2% to about 4%. In certain embodiments, the polymer composition comprises a combination of a first GelMA mixture and a second GelMA mixture, wherein the total GelMA in the polymer composition is from about 3% to about 5%. In certain embodiments, the polymer composition comprises a combination of a first GelMA mixture and a second GelMA mixture, wherein the total GelMA in the polymer composition is from about 3% to about 4%.

In certain embodiments, the polymer composition comprises from about 0.5% to about 3% of the first GelMA mixture. In certain embodiments, the polymer composition comprises from about 0.5% to about 3% of the second GelMA mixture. In certain embodiments, the polymer composition comprises from about 0.5% to about 3% of the first GelMA mixture, and about 0.5% to about 3% of the second GelMA mixture. In certain embodiments, the polymer composition comprises from about 0.5% to about 1.5% of the first GelMA mixture, and about 2% to about 3% of the second GelMA mixture. In certain embodiments, the polymer composition comprises from about 2% to about 3% of the first GelMA mixture, and about 0.5% to about 1.5% of the second GelMA mixture. In certain embodiments, the polymer composition comprises about 1% of the first GelMA mixture and about 2.5% of the second GelMA mixture.

In certain embodiments, the first GelMA mixture includes GelMA having a high average molecular weight (e.g., from 140-180 kDa, or about 160 kDa). In certain embodiments, the first GelMA mixture includes GelMA having a low degree of methacrylation (DOM) (e.g., from 5% to 40%, or about 10%). In certain embodiments, the first GelMA mixture includes GelMA having a high average molecular weight (e.g., from 140-180 kDa, or about 160 kDa) and a low (DOM) (e.g., from 5% to 40%, or about 10%). In certain embodiments, the first GelMA mixture includes GelMA having an average molecular weight from 140-180 kDa and a DOM from 5% to 40%. In certain embodiments, the first GelMA mixture includes GelMA having an average molecular weight from 140-180 kDa and a DOM from 5% to 20%. In certain embodiments, the first GelMA mixture includes GelMA having an average molecular weight of about 160 kDa and a DOM of about 10%.

In certain embodiments, the second GelMA mixture includes GelMA having a low average molecular weight (e.g., from 75-115 kDa, or about 90 kDa). In certain embodiments, the second GelMA mixture includes GelMA having a high degree of methacrylation (DOM) (e.g., from 50% to 80%, or about 60%). In certain embodiments, the second GelMA mixture includes GelMA having a low average molecular weight (e.g., from 75-115 kDa, or about 90 kDa) and a high (DOM) (e.g., from 50% to 80%, or about 60%). In certain embodiments, the second GelMA mixture includes GelMA having an average molecular weight from 75-115 kDa and a DOM from 50% to 80%. In certain embodiments, the second GelMA mixture includes GelMA having an average molecular weight from 80-115 kDa and a DOM from 50% to 70%. In certain embodiments, the second GelMA mixture includes GelMA having an average molecular weight of about 90 kDa and a DOM of about 60%.

In certain embodiments, the first GelMA mixture includes GelMA having a high average molecular weight (e.g., from 140-180 kDa, or about 160 kDa) and a low (DOM) (e.g., from 5% to 40%, or about 10%); and the second GelMA mixture includes GelMA having a low average molecular weight (e.g., from 75-115 kDa, or about 90 kDa) and a high (DOM) (e.g., from 50% to 80%, or about 60%). In certain embodiments, the first GelMA mixture includes GelMA having an average molecular weight from 140-180 kDa and a DOM from 5% to 40%; and the second GelMA mixture includes GelMA having an average molecular weight from 75-115 kDa and a DOM from 50% to 80%. In certain embodiments, the first GelMA mixture includes GelMA having an average molecular weight from 140-180 kDa and a DOM from 5% to 20%; and the second GelMA mixture includes GelMA having an average molecular weight from 80-100 kDa and a DOM from 50% to 70%. In certain embodiments, the first GelMA mixture includes GelMA having an average molecular weight of about 160 kDa and a DOM of about 10%; and the second GelMA mixture includes GelMA having an average molecular weight of about 90 kDa and a DOM of about 60%.

In certain embodiments, the polymer composition comprises from about 0.5% to about 3% w/v of a first GelMA mixture, which includes GelMA having a high average molecular weight (e.g., from 140-180 kDa, or about 160 kDa) and a low (DOM) (e.g., from 5% to 40%, or about 10%). In certain embodiments, the polymer composition comprises from about 0.5% to about 3% w/v of a second GelMA mixture, which includes GelMA having a low average molecular weight (e.g., from 75-115 kDa, or about 90 kDa) and a high (DOM) (e.g., from 50% to 80%, or about 60%). In certain embodiments, the polymer composition comprises: from about 0.5% to about 3% w/v of a first GelMA mixture, which includes GelMA having a high average molecular weight (e.g., from 140-180 kDa, or about 160 kDa) and a low (DOM) (e.g., from 5% to 40%, or about 10%); and from about 0.5% to about 3% w/v of a second GelMA mixture, which includes GelMA having a low average molecular weight (e.g., from 75-115 kDa, or about 90 kDa) and a high (DOM) (e.g., from 50% to 80%, or about 60%).

In certain embodiments, the polymer composition comprises: from about 0.5% to about 1.5% w/v of a first GelMA mixture, which includes GelMA having an average molecular weight from 140-180 kDa and a DOM from 5% to 40%; and from about 2% to about 3% w/v of a second GelMA mixture which includes GelMA having an average molecular weight from 75-115 kDa and a DOM from 50% to 80%. In certain embodiments, the polymer composition comprises: from about 0.5% to about 1.5% w/v of a first GelMA mixture, which includes GelMA having an average molecular weight from 140-180 kDa and a DOM from 5% to 20%; and from about 2% to about 3% w/v of a second GelMA mixture which includes GelMA having an average molecular weight from 85-100 kDa and a DOM from 50% to 70%.

In certain embodiments, the polymer composition comprises: about 1% w/v of a first GelMA mixture which includes GelMA having an average molecular weight of about 160 kDa and a DOM of about 10%; and about 2.5% w/v of a second GelMA mixture which includes GelMA having an average molecular weight of about 90 kDa and a DOM of about 60%.

In certain embodiments, the polymer composition comprises a combination of GelMA having about 10% DoM (e.g., GelMA 160/10) and GelMA having about 80% DoM (e.g., GelMA 160/80). In certain embodiments, the polymer composition comprises a combination of GelMA having about 10% DoM and GelMA having about 80% DoM, wherein the polymer composition comprises about 4-10% w/v of GelMA. In certain embodiments, the polymer composition comprises a combination of GelMA having about 10% DoM (e.g., GelMA 160/10) and GelMA having about 80% DoM (e.g., GelMA 160/80), wherein the ratio of 10% DoM GelMA and 80% DoM GelMA is from about 1:9 to about 9:1. In certain embodiments, the ratio of 10% DoM GelMA and 80% DoM GelMA is about 1:9. In certain embodiments, the ratio of 10% DoM GelMA and 80% DoM GelMA is about 2:8 (i.e., about 1:4). In certain embodiments, the ratio of 10% DoM GelMA and 80% DoM GelMA is about 3:7. In certain embodiments, the ratio of 10% DoM GelMA and 80% DoM GelMA is about 4:6 (i.e., about 2:3). In certain embodiments, the ratio of 10% DoM GelMA and 80% DoM GelMA is about 5:5 (i.e., about 1:1). In certain embodiments, the ratio of 10% DoM GelMA and 80% DoM GelMA is about 4:6 (i.e., about 2:3). In certain embodiments, the ratio of 10% DoM GelMA and 80% DoM GelMA is about 3:7. In certain embodiments, the ratio of 10% DoM GelMA and 80% DoM GelMA is about 2:8 (i.e., about 1:4). In certain embodiments, the ratio of 10% DoM GelMA and 80% DoM GelMA is about 1:9.

In certain embodiments, the polymer composition comprises glycidyl methacrylate-functionalized GelMA having about 45% DoM (e.g., GelMA 160/45). In certain embodiments, the polymer composition comprises glycidyl methacrylate-functionalized GelMA having about 45% DoM and 160 kDa (GelMA 160/45). In certain embodiments, the polymer composition comprises glycidyl methacrylate-functionalized GelMA having about 45% DoM and 90 kDa (GelMA 90/45).

In certain embodiments, the polymer composition comprises MeHA having about 500 kDa molecular weight (MW). In certain embodiments, the polymer composition comprises MeHA having about 30% degree of methacrylation (DoM). In certain embodiments, the polymer composition comprises PEGDA formed from about 35 kDa PEG materials. In certain embodiments, the polymer composition comprises PEGDA formed from about 2 kDa PEG materials.

In certain embodiments, the polymer composition comprises a poloxamer surfactant (e.g., Poloxamer 407). In certain embodiments, the polymer composition comprises about 0.1-0.5% w/v (e.g., about 0.2% w/v) of a poloxamer surfactant (e.g., Poloxamer 407). In certain embodiments, the polymer composition comprises a tyloxapol surfactant. In certain embodiments, the polymer composition comprises about 0.1-0.5% w/v (e.g., about 0.1% w/v) of a tyloxapol surfactant.

In certain embodiments, the polymer composition comprises about 4% w/v of chemically-modified gelatin (e.g., GelMA). In certain embodiments, the polymer composition comprises about 4% w/v of chemically-modified gelatin (e.g., GelMA); about 1-1.5% w/v of chemically modified hyaluronic acid (e.g., MeHA); and about 0.1-5% w/v of chemically modified PEG (e.g., PEGDA). In certain embodiments, the polymer composition comprises about 4% w/v of chemically-modified gelatin (e.g., GelMA); about 1% w/v of chemically modified hyaluronic acid (e.g., MeHA); and about 0.1-5% w/v of chemically modified PEG (e.g., PEGDA). In certain embodiments, the polymer composition comprises about 4% w/v of chemically-modified gelatin (e.g., GelMA); about 1% w/v of chemically modified hyaluronic acid (e.g., MeHA); and about 0.1% w/v of chemically modified PEG (e.g., PEGDA). In certain embodiments, the polymer composition comprises about 4% w/v of chemically-modified gelatin (e.g., GelMA); about 1% w/v of chemically modified hyaluronic acid (e.g., MeHA); and about 0.5% w/v of chemically modified PEG (e.g., PEGDA). In certain embodiments, the polymer composition comprises about 4% w/v of chemically-modified gelatin (e.g., GelMA); about 1% w/v of chemically modified hyaluronic acid (e.g., MeHA); and about 0.67% w/v of chemically modified PEG (e.g., PEGDA). In certain embodiments, the polymer composition comprises about 4% w/v of chemically-modified gelatin (e.g., GelMA); about 1% w/v of chemically modified hyaluronic acid (e.g., MeHA); and about 1.0% w/v of chemically modified PEG (e.g., PEGDA). In certain embodiments, the polymer composition comprises about 4% w/v of chemically-modified gelatin (e.g., GelMA); about 1.5% w/v of chemically modified hyaluronic acid (e.g., MeHA); and about 0.1-5% w/v of chemically modified PEG (e.g., PEGDA). In certain embodiments, the polymer composition comprises about 4% w/v of chemically-modified gelatin (e.g., GelMA); about 1.5% w/v of chemically modified hyaluronic acid (e.g., MeHA); and about 0.1% w/v of chemically modified PEG (e.g., PEGDA). In certain embodiments, the polymer composition comprises about 4% w/v of chemically-modified gelatin (e.g., GelMA); about 1.5% w/v of chemically modified hyaluronic acid (e.g., MeHA); and about 0.5% w/v of chemically modified PEG (e.g., PEGDA). In certain embodiments, the polymer composition comprises about 4% w/v of chemically-modified gelatin (e.g., GelMA); about 1.5% w/v of chemically modified hyaluronic acid (e.g., MeHA); and about 0.67% w/v of chemically modified PEG (e.g., PEGDA). In certain embodiments, the polymer composition comprises about 4% w/v of chemically-modified gelatin (e.g., GelMA); about 1.5% w/v of chemically modified hyaluronic acid (e.g., MeHA); and about 1.0% w/v of chemically modified PEG (e.g., PEGDA).

In certain embodiments, the polymer composition comprises about 5% w/v of chemically-modified gelatin (e.g., GelMA). In certain embodiments, the polymer composition comprises about 5% w/v of chemically-modified gelatin (e.g., GelMA); about 1-1.5% w/v of chemically modified hyaluronic acid (e.g., MeHA); and about 0.1-5% w/v of chemically modified PEG (e.g., PEGDA). In certain embodiments, the polymer composition comprises about 5% w/v of chemically-modified gelatin (e.g., GelMA); about 1% w/v of chemically modified hyaluronic acid (e.g., MeHA); and about 0.1-5% w/v of chemically modified PEG (e.g., PEGDA). In certain embodiments, the polymer composition comprises about 5% w/v of chemically-modified gelatin (e.g., GelMA); about 1% w/v of chemically modified hyaluronic acid (e.g., MeHA); and about 0.1% w/v of chemically modified PEG (e.g., PEGDA). In certain embodiments, the polymer composition comprises about 5% w/v of chemically-modified gelatin (e.g., GelMA); about 1% w/v of chemically modified hyaluronic acid (e.g., MeHA); and about 0.5% w/v of chemically modified PEG (e.g., PEGDA). In certain embodiments, the polymer composition comprises about 5% w/v of chemically-modified gelatin (e.g., GelMA); about 1% w/v of chemically modified hyaluronic acid (e.g., MeHA); and about 0.67% w/v of chemically modified PEG (e.g., PEGDA). In certain embodiments, the polymer composition comprises about 5% w/v of chemically-modified gelatin (e.g., GelMA); about 1% w/v of chemically modified hyaluronic acid (e.g., MeHA); and about 1.0% w/v of chemically modified PEG (e.g., PEGDA). In certain embodiments, the polymer composition comprises about 5% w/v of chemically-modified gelatin (e.g., GelMA); about 1.5% w/v of chemically modified hyaluronic acid (e.g., MeHA); and about 0.1-5% w/v of chemically modified PEG (e.g., PEGDA). In certain embodiments, the polymer composition comprises about 5% w/v of chemically-modified gelatin (e.g., GelMA); about 1.5% w/v of chemically modified hyaluronic acid (e.g., MeHA); and about 0.1% w/v of chemically modified PEG (e.g., PEGDA). In certain embodiments, the polymer composition comprises about 5% w/v of chemically-modified gelatin (e.g., GelMA); about 1.5% w/v of chemically modified hyaluronic acid (e.g., MeHA); and about 0.5% w/v of chemically modified PEG (e.g., PEGDA). In certain embodiments, the polymer composition comprises about 5% w/v of chemically-modified gelatin (e.g., GelMA); about 1.5% w/v of chemically modified hyaluronic acid (e.g., MeHA); and about 0.67% w/v of chemically modified PEG (e.g., PEGDA). In certain embodiments, the polymer composition comprises about 5% w/v of chemically-modified gelatin (e.g., GelMA); about 1.5% w/v of chemically modified hyaluronic acid (e.g., MeHA); and about 1.0% w/v of chemically modified PEG (e.g., PEGDA).

In certain embodiments, the polymer composition comprises about 10% w/v of chemically-modified gelatin (e.g., GelMA). In certain embodiments, the polymer composition comprises about 10% w/v of chemically-modified gelatin (e.g., GelMA); about 1-1.5% w/v of chemically modified hyaluronic acid (e.g., MeHA); and about 0.1-5% w/v of chemically modified PEG (e.g., PEGDA). In certain embodiments, the polymer composition comprises about 10% w/v of chemically-modified gelatin (e.g., GelMA); about 1% w/v of chemically modified hyaluronic acid (e.g., MeHA); and about 0.1-5% w/v of chemically modified PEG (e.g., PEGDA). In certain embodiments, the polymer composition comprises about 10% w/v of chemically-modified gelatin (e.g., GelMA); about 1% w/v of chemically modified hyaluronic acid (e.g., MeHA); and about 0.1% w/v of chemically modified PEG (e.g., PEGDA). In certain embodiments, the polymer composition comprises about 10% w/v of chemically-modified gelatin (e.g., GelMA); about 1% w/v of chemically modified hyaluronic acid (e.g., MeHA); and about 0.5% w/v of chemically modified PEG (e.g., PEGDA). In certain embodiments, the polymer composition comprises about 10% w/v of chemically-modified gelatin (e.g., GelMA); about 1% w/v of chemically modified hyaluronic acid (e.g., MeHA); and about 0.67% w/v of chemically modified PEG (e.g., PEGDA). In certain embodiments, the polymer composition comprises about 10% w/v of chemically-modified gelatin (e.g., GelMA); about 1% w/v of chemically modified hyaluronic acid (e.g., MeHA); and about 1.0% w/v of chemically modified PEG (e.g., PEGDA). In certain embodiments, the polymer composition comprises about 10% w/v of chemically-modified gelatin (e.g., GelMA); about 1.5% w/v of chemically modified hyaluronic acid (e.g., MeHA); and about 0.1-5% w/v of chemically modified PEG (e.g., PEGDA). In certain embodiments, the polymer composition comprises about 10% w/v of chemically-modified gelatin (e.g., GelMA); about 1.5% w/v of chemically modified hyaluronic acid (e.g., MeHA); and about 0.1% w/v of chemically modified PEG (e.g., PEGDA). In certain embodiments, the polymer composition comprises about 10% w/v of chemically-modified gelatin (e.g., GelMA); about 1.5% w/v of chemically modified hyaluronic acid (e.g., MeHA); and about 0.5% w/v of chemically modified PEG (e.g., PEGDA). In certain embodiments, the polymer composition comprises about 10% w/v of chemically-modified gelatin (e.g., GelMA); about 1.5% w/v of chemically modified hyaluronic acid (e.g., MeHA); and about 0.67% w/v of chemically modified PEG (e.g., PEGDA). In certain embodiments, the polymer composition comprises about 10% w/v of chemically-modified gelatin (e.g., GelMA); about 1.5% w/v of chemically modified hyaluronic acid (e.g., MeHA); and about 1.0% w/v of chemically modified PEG (e.g., PEGDA).

In certain embodiments, the polymer composition comprises about 20% w/v of chemically-modified gelatin (e.g., GelMA). In certain embodiments, the polymer composition comprises about 20% w/v of chemically-modified gelatin (e.g., GelMA); about 1-1.5% w/v of chemically modified hyaluronic acid (e.g., MeHA); and about 0.1-5% w/v of chemically modified PEG (e.g., PEGDA). In certain embodiments, the polymer composition comprises about 20% w/v of chemically-modified gelatin (e.g., GelMA); about 1% w/v of chemically modified hyaluronic acid (e.g., MeHA); and about 0.1-5% w/v of chemically modified PEG (e.g., PEGDA). In certain embodiments, the polymer composition comprises about 20% w/v of chemically-modified gelatin (e.g., GelMA); about 1% w/v of chemically modified hyaluronic acid (e.g., MeHA); and about 0.1% w/v of chemically modified PEG (e.g., PEGDA). In certain embodiments, the polymer composition comprises about 20% w/v of chemically-modified gelatin (e.g., GelMA); about 1% w/v of chemically modified hyaluronic acid (e.g., MeHA); and about 0.5% w/v of chemically modified PEG (e.g., PEGDA). In certain embodiments, the polymer composition comprises about 20% w/v of chemically-modified gelatin (e.g., GelMA); about 1% w/v of chemically modified hyaluronic acid (e.g., MeHA); and about 0.67% w/v of chemically modified PEG (e.g., PEGDA). In certain embodiments, the polymer composition comprises about 20% w/v of chemically-modified gelatin (e.g., GelMA); about 1% w/v of chemically modified hyaluronic acid (e.g., MeHA); and about 1.0% w/v of chemically modified PEG (e.g., PEGDA). In certain embodiments, the polymer composition comprises about 20% w/v of chemically-modified gelatin (e.g., GelMA); about 1.5% w/v of chemically modified hyaluronic acid (e.g., MeHA); and about 0.1-5% w/v of chemically modified PEG (e.g., PEGDA). In certain embodiments, the polymer composition comprises about 20% w/v of chemically-modified gelatin (e.g., GelMA); about 1.5% w/v of chemically modified hyaluronic acid (e.g., MeHA); and about 0.1% w/v of chemically modified PEG (e.g., PEGDA). In certain embodiments, the polymer composition comprises about 20% w/v of chemically-modified gelatin (e.g., GelMA); about 1.5% w/v of chemically modified hyaluronic acid (e.g., MeHA); and about 0.5% w/v of chemically modified PEG (e.g., PEGDA). In certain embodiments, the polymer composition comprises about 20% w/v of chemically-modified gelatin (e.g., GelMA); about 1.5% w/v of chemically modified hyaluronic acid (e.g., MeHA); and about 0.67% w/v of chemically modified PEG (e.g., PEGDA). In certain embodiments, the polymer composition comprises about 20% w/v of chemically-modified gelatin (e.g., GelMA); about 1.5% w/v of chemically modified hyaluronic acid (e.g., MeHA); and about 1.0% w/v of chemically modified PEG (e.g., PEGDA).

In certain embodiments, the polymer composition comprises about 4-20% w/v of chemically-modified gelatin (e.g., GelMA); and about 0-5% w/v of chemically modified PEG (e.g., PEGDA). In certain embodiments, the polymer composition comprises about 4-10% w/v of chemically-modified gelatin (e.g., GelMA); and about 0.1-5% w/v of chemically modified PEG (e.g., PEGDA). In certain embodiments, the polymer composition comprises about 4% w/v of chemically-modified gelatin (e.g., GelMA); and about 0.1-5% w/v of chemically modified PEG (e.g., PEGDA). In certain embodiments, the polymer composition comprises about 4% w/v of chemically-modified gelatin (e.g., GelMA); and about 0.1% w/v of chemically modified PEG (e.g., PEGDA). In certain embodiments, the polymer composition comprises about 4% w/v of chemically-modified gelatin (e.g., GelMA); and about 0.5% w/v of chemically modified PEG (e.g., PEGDA). In certain embodiments, the polymer composition comprises about 4% w/v of chemically-modified gelatin (e.g., GelMA); and about 0.67% w/v of chemically modified PEG (e.g., PEGDA). In certain embodiments, the polymer composition comprises about 4% w/v of chemically-modified gelatin (e.g., GelMA); and about 1.0% w/v of chemically modified PEG (e.g., PEGDA). In certain embodiments, the polymer composition comprises about 5% w/v of chemically-modified gelatin (e.g., GelMA); and about 0.1% w/v of chemically modified PEG (e.g., PEGDA). In certain embodiments, the polymer composition comprises about 5% w/v of chemically-modified gelatin (e.g., GelMA); and about 0.5% w/v of chemically modified PEG (e.g., PEGDA). In certain embodiments, the polymer composition comprises about 5% w/v of chemically-modified gelatin (e.g., GelMA); and about 0.67% w/v of chemically modified PEG (e.g., PEGDA). In certain embodiments, the polymer composition comprises about 5% w/v of chemically-modified gelatin (e.g., GelMA); and about 1.0% w/v of chemically modified PEG (e.g., PEGDA). In certain embodiments, the polymer composition comprises about 7% w/v of chemically-modified gelatin (e.g., GelMA); and about 0.1% w/v of chemically modified PEG (e.g., PEGDA). In certain embodiments, the polymer composition comprises about 7% w/v of chemically-modified gelatin (e.g., GelMA); and about 0.5% w/v of chemically modified PEG (e.g., PEGDA). In certain embodiments, the polymer composition comprises about 7% w/v of chemically-modified gelatin (e.g., GelMA); and about 0.67% w/v of chemically modified PEG (e.g., PEGDA). In certain embodiments, the polymer composition comprises about 7% w/v of chemically-modified gelatin (e.g., GelMA); and about 1.0% w/v of chemically modified PEG (e.g., PEGDA). In certain embodiments, the polymer composition comprises about 10% w/v of chemically-modified gelatin (e.g., GelMA); and about 0.1% w/v of chemically modified PEG (e.g., PEGDA). In certain embodiments, the polymer composition comprises about 10% w/v of chemically-modified gelatin (e.g., GelMA); and about 0.5% w/v of chemically modified PEG (e.g., PEGDA). In certain embodiments, the polymer composition comprises about 10% w/v of chemically-modified gelatin (e.g., GelMA); and about 0.67% w/v of chemically modified PEG (e.g., PEGDA). In certain embodiments, the polymer composition comprises about 10% w/v of chemically-modified gelatin (e.g., GelMA); and about 1.0% w/v of chemically modified PEG (e.g., PEGDA). In certain embodiments, the polymer composition comprises about 20% w/v of chemically-modified gelatin (e.g., GelMA); and about 0.1% w/v of chemically modified PEG (e.g., PEGDA). In certain embodiments, the polymer composition comprises about 20% w/v of chemically-modified gelatin (e.g., GelMA); and about 0.5% w/v of chemically modified PEG (e.g., PEGDA). In certain embodiments, the polymer composition comprises about 20% w/v of chemically-modified gelatin (e.g., GelMA); and about 0.67% w/v of chemically modified PEG (e.g., PEGDA). In certain embodiments, the polymer composition comprises about 20% w/v of chemically-modified gelatin (e.g., GelMA); and about 1.0% w/v of chemically modified PEG (e.g., PEGDA).

In certain embodiments, the polymer composition comprises: about 4% GelMA (10-40% DoM); and about 1% PEGDA (35 kDa). In certain embodiments, the polymer composition comprises: about 2% GelMA (10-40% DoM); about 2% Gelatin; and about 1% PEGDA (35 kDa). In certain embodiments, the polymer composition comprises: about 4% Gelatin Acrylate (10-40% DoM); and about 1% PEGDA (35 kDa). In certain embodiments, the polymer composition comprises: about 2% Gelatin Acrylate (10-40% DoM); about 2% Gelatin; and about 1% PEGDA (35 kDa). In certain embodiments, the polymer composition comprises: about 4% GelMA (10-40% DoM); about 1% PEGDA (35 kDa); and about 1-20% PEG Methyl Ether Acrylate (35 kDa). In certain embodiments, the polymer composition comprises: about 4% GelMA (10-40% DoM); about 1% HAMA (500 kDa, 5-40% DoM); and about 1% PEGDA (35 kDa). In certain embodiments, the polymer composition comprises: about 2% GelMA (10-40% DoM); about 2% Gelatin; about 1% HAMA (500 kDa, 5-40% DoM); and about 1% PEGDA (35 kDa). In certain embodiments, the polymer composition comprises: about 4% Gelatin Acrylate (10-40% DoM); about 1% HAMA (500 kDa, 5-40% DoM); and about 1% PEGDA (35 kDa). In certain embodiments, the polymer composition comprises: about 2% Gelatin Acrylate (10-40% DoM); about 2% Gelatin; about 1% HAMA (500 kDa, 5-40% DoM); and about 1% PEGDA (35 kDa). In certain embodiments, the polymer composition comprises: about 4% GelMA (10-40% DoM); about 1% HAMA (500 kDa, 5-40% DoM); about 1% PEGDA (35 kDa); and about 1-20% PEG Methyl Ether Acrylate (35 kDa). In certain embodiments, the polymer composition comprises: about 5-20% GelMA (10-40% DoM). In certain embodiments, the polymer composition comprises: about 5-20% GelMA (10-40% DoM); and about 1% HAMA (500 kDa, 5-40% DoM).

In certain embodiments, the polymer composition comprises: about 4% GelMA (80% DoM); about 1% PEGDA (2 kDa); and about 0.2% (w/v) of a poloxamer surfactant (e.g., Poloxamer 407); optionally with an active agent (e.g., corticosteroid). In certain embodiments, the polymer composition comprises: about 4% GelMA (40% DoM); about 1% PEGDA (35 kDa); and about 0.2% (w/v) of a poloxamer surfactant (e.g., Poloxamer 407); optionally with an active agent (e.g., corticosteroid). In certain embodiments, the polymer composition comprises: about 4% GelMA (10% DoM); about 1% PEGDA (35 kDa); and about 0.2% (w/v) of a poloxamer surfactant (e.g., Poloxamer 407); optionally with an active agent (e.g., corticosteroid). In certain embodiments, the polymer composition comprises: about 20% GelMA (40% DoM); and about 0.2% (w/v) of a poloxamer surfactant (e.g., Poloxamer 407); optionally with an active agent (e.g., corticosteroid).

### Chemically Modified Gelatin

Gelatin is a naturally-derived, biocompatible mixture of peptides and proteins derived from collagen, which is a primary structural component of animal tissue (including ocular tissue, bones, and skin). Natural matrix peptides and proteins (e.g., denatured collagen) which can be used in the production of gelatin materials of the present disclosure can include gelatin components derived from animals including, but not limited to, pig, cow, horse, chicken, and fish. In certain embodiments, gelatin materials can be derived from connective tissue proteins, such as collagen. In certain embodiments, gelatin materials can be derived from bone, skin, or ocular tissues. In certain embodiments, gelatin materials can be prepared by acid hydrolysis and/or base hydrolysis of connective tissue proteins (e.g., collagen).

In certain embodiments, polymer compositions of the present disclosure comprises a chemically-modified gelatin. In certain embodiments, the polymer compositions comprises acrylated gelatin. In certain embodiments, the polymer compositions comprises gelatin methacryloyl (i.e., GelMA). In certain embodiments, a chemically modified gelatin can be included in precursor polymer compositions of the present disclosure. In certain embodiments, the chemically-modified gelatin comprises a photo-crosslinkable derivative of gelatin. In certain embodiments, the chemically modified gelatin can be modified with an acrylic anhydride or acryloyl chloride (substituted or unsubstituted) to form an acryloyl-substituted gelatin. In certain embodiments, the chemically modified gelatin can be modified with one or more crosslinkable groups selected from methyl acrylate, ethyl acrylate, propyl acrylate, methyl methacrylate, ethyl methacrylate, methacryloyl, catechol, ethylene oxide, or propylene oxide. In certain embodiments, the chemically modified gelatin can be modified with methacrylic anhydride (MA) (also known as methacryloyl anhydride) to form a methacryloyl-substituted gelatin (commonly referred to as gelatin methacryloyl, or GelMA). FIG. TA provides an example of a reaction in which gelatin is modified with methacrylic anhydride to form a methacryloyl-substituted gelatin (GelMA).

In certain embodiments, acryloyl modification of gelatin can be performed by a synthesis reaction of gelatin with a functionalizing compound which comprises an acrylate group. In certain embodiments, methacryloyl modification of gelatin can be performed by a synthesis reaction of gelatin with methacrylic anhydride, methacryloyl chloride, 2-isocyanatoethyl methacrylate, 2-hydroxyethyl methacrylate, glycidyl methacrylate, methacrylic acid N-hydroxysuccinimide ester, allyl methacrylate, vinyl methacrylate, bis(2-methacryloyl)oxyethyl disulfide, 2-hydroxy-5-N-methacrylamidobenzoic acid, or combinations thereof.

As used herein, the terms “acryloyl-substituted gelatin” and “acrylated gelatin” can describe a gelatin having free amines (e.g., lysine, arginine, asparagine, or glutamine side chains) and/or free hydroxyls (e.g., serine, threonine, aspartic acid or glutamic acid side chains) that have been substituted with at least one acryloyl group. Generally, an acryloyl group is an a,b-unsaturated carbonyl compound represented by the formula H₂C═CR′—C(═O)—R, where R′ can be, but is not limited to: hydrogen, halogen, hydroxyl, C₁-C₅ alkoxy, C₁-C₅ alkyl, C₃-C₈ cycloalkyl, C₁-C₅ heteroalkyl, C₃-C₈ heterocycloalkyl, aryl, heteroaryl or amino group, each being optionally substituted with halogen, C₁-C₅ alkoxy, C₁-C₅ alkyl, C₃-C₈ cycloalkyl, C₁-C₅ heteroalkyl, C₃-C₈ heterocycloalkyl, aryl, heteroaryl or amino group. For acryloyl-substituted gelatins of the present disclosure, the R group represents a terminal amine and/or hydroxyl group on the gelatin which is subject to the acryloyl functionalization.

In certain embodiments, the R′ group of the acryloyl moiety is methyl, commonly referred to as a methacryloyl group. As used herein, the terms “methacryloyl-substituted gelatin”, “gelatin methacryloyl”, and “GelMA” can describe a gelatin having free amines (e.g., lysine, arginine, asparagine, or glutamine side chains) and/or free hydroxyls (e.g., serine, threonine, aspartic acid or glutamic acid side chains) that have been substituted with at least one methacryloyl group, such as methacrylamide groups (from free amines on the gelatin) and/or a methacrylate groups (from free hydroxyls on the gelatin).

In certain embodiments, a chemically-modified gelatin (e.g., GelMA) can be present in the polymer composition at a concentration from about 1% to about 60% weight per volume (w/v). In certain embodiments, a chemically-modified gelatin (e.g., GelMA) can be present in the polymer composition at a weight per volume concentration (w/v) of about 0.5%, about 1%, about 2%, about 3%, about 4%, about 5%, about 6%, about 7%, about 8% about 9%, about 10%, about 11% about 12%, about 13%, about 14%, about 15%, about 16%, about 17%, about 18% about 19%, about 20%, about 21% about 22%, about 23%, about 24%, about 25%, about 26%, about 27%, about 28% about 29%, about 30%, about 31% about 32%, about 33%, about 34%, about 35%, about 36%, about 37%, about 38% about 39%, about 40%, about 41% about 42%, about 43%, about 44%, about 45%, about 46%, about 47%, about 48% about 49%, about 50%, about 51% about 52%, about 53%, about 54%, about 55%, about 56%, about 57%, about 58% about 59%, or about 60%. In certain embodiments, a chemically-modified gelatin (e.g., GelMA) can be present in the polymer composition at a weight per volume concentration (w/v) of from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, about 45-50%, about 51-53%, about 53-56%, about 56-60%, about 50-60%, about 50-55%, or about 55-60%.

In certain embodiments, a polymer compositions comprises acrylated gelatin (i.e., GelMA) with a degree of acryloyl substitution (i.e., methacryloyl functionalization). As used herein, the term “degree of acryloyl substitution” can describe the percentage of free amines and hydroxyls in a gelatin that have been substituted with acryloyl groups. As used herein, the term “degree of methacryloyl substitution” can describe the percentage of free amines and hydroxyls in a gelatin that have been substituted with methacryloyl groups. In certain embodiments, a polymer composition comprises acrylated gelatin with a degree of acryloyl substitution of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%. In certain embodiments, a polymer composition comprises acrylated gelatin with a degree of acryloyl substitution from about 10-99%. In certain embodiments, the degree of acryloyl substitution is from about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-99%. In certain embodiments, a polymer compositions comprises GelMA with a degree of methacryloyl substitution of from about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-99%.

In certain embodiments, a polymer composition comprises GelMA with a degree of methacrylamide substitution (i.e., methacrylamide functionalization). In certain embodiments, a polymer composition comprises GelMA with a degree of methacrylamide substitution of at least about 20%, about 25%, about 30%, about 35%, about 40%, about 45%, about 50%, about 55%, about 60%, about 65%, about 70%, about 75%, about 80%, about 85%, or at least about 90%. In certain embodiments, a polymer composition comprises GelMA with a degree of methacrylamide substitution from about 20-90%. In certain embodiments, the degree of methacrylamide substitution is from about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, or about 85-90%. In certain embodiments, the degree of methacrylamide substitution can be measured using proton nuclear magnetic resonance. In certain embodiments, the degree of methacrylamide substitution can be measured using a fluoraldehyde assay.

In certain embodiments, a polymer composition comprises GelMA with a degree of methacrylate substitution (i.e., methacrylate functionalization). In certain embodiments, a polymer composition comprises GelMA with a degree of methacrylate substitution of at least about 20%, about 25%, about 30%, about 35%, about 40%, about 45%, about 50%, about 55%, about 60%, about 65%, about 70%, about 75%, about 80%, about 85%, or at least about 90%. In certain embodiments, a polymer composition comprises GelMA with a degree of methacrylate substitution from about 20-90%. In certain embodiments, the degree of methacrylate substitution is from about 1-5%, about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, or about 85-90%. In certain embodiments, the degree of methacrylate substitution can be measured using proton nuclear magnetic resonance. In certain embodiments, the degree of methacrylate substitution can be measured using a Fe(III)-hydroxamic acid-based assay. In certain embodiments, measurement of the degree of methacrylate substitution can include an aminolysis reaction (e.g., by exposure to a hydroxylamine solution) to convert methacrylate groups into N-hydroxymethacrylamide groups.

In certain embodiments, a polymer composition comprises GelMA with a degree of methacrylamide substitution and with a degree of methacrylate substitution. In certain embodiments, the ratio of methacrylamide substitution to methacrylate substitution in the GelMA is from about 1:1 to 99:1. In some embodiments, the ratio of methacrylamide substitution to methacrylate substitution is from about 1:1 to 2:1, about 2:1 to 3:1, about 3:1 to 4:1, about 4:1 to 5:1, about 1:1 to 5:1, about 5:1 to 10:1, about 10:1 to 15:1, about 15:1 to 20:1, about 20:1 to 25:1, about 25:1 to 30:1, about 30:1 to 35:1, about 35:1 to 40:1, about 40:1 to 45:1, about 45:1 to 50:1, about 50:1 to 55:1, about 55:1 to 60:1, about 60:1 to 65:1, about 65:1 to 70:1, about 70:1 to 75:1, about 75:1 to 80:1, about 80:1 to 85:1, about 85:1 to 90:1, about 90:1 to 95:1, or about 95:1 to 99:1. In certain embodiments, the ratio of methacrylate substitution to methacrylamide substitution in the GelMA is from about 1:1 to 99:1. In some embodiments, the ratio of methacrylate substitution to methacrylamide substitution is from about 1:1 to 2:1, about 2:1 to 3:1, about 3:1 to 4:1, about 4:1 to 5:1, about 1:1 to 5:1, about 5:1 to 10:1, about 10:1 to 15:1, about 15:1 to 20:1, about 20:1 to 25:1, about 25:1 to 30:1, about 30:1 to 35:1, about 35:1 to 40:1, about 40:1 to 45:1, about 45:1 to 50:1, about 50:1 to 55:1, about 55:1 to 60:1, about 60:1 to 65:1, about 65:1 to 70:1, about 70:1 to 75:1, about 75:1 to 80:1, about 80:1 to 85:1, about 85:1 to 90:1, about 90:1 to 95:1, or about 95:1 to 99:1.

In certain embodiments, the polymer composition comprises GelMA with a methacryloyl modification of gelatin performed by a reaction of gelatin with methacrylic anhydride. In certain embodiments, the polymer composition comprises GelMA with a methacryloyl modification of gelatin performed by a reaction of gelatin with glycidyl methacrylate.

In certain embodiments, a gelatin can be functionalized with anchoring integrins and/or proteins (e.g., proteins which bind to the surface proteins of a target surface). Said functionalization can occur with poly(ethylene glycol) (PEG) or other polymeric linkers between the gelatin and integrin and/or protein.

### Chemically Modified Hyaluronic Acid

Hyaluronic acid (HA) is a viscoelastic and biocompatible glycosaminoglycan which is naturally present in the cornea and other tissues. In certain embodiments, polymer compositions of the present disclosure comprises a chemically-modified hyaluronic acid (HA). In certain embodiments, a polymer composition comprises an acryloyl-substituted hyaluronic acid. In certain embodiments, a polymer composition comprises methacrylated hyaluronic acid (MeHA). In certain embodiments, a chemically modified HA can be included in precursor polymer compositions of the present disclosure. In certain embodiments, the chemically-modified HA comprises a photo-crosslinkable derivative of HA. In certain embodiments, the chemically-modified HA comprises methacrylated hyaluronic acid (MeHA). In certain embodiments, the chemically-modified HA comprises a methacrylated hyaluronic acid (MeHA) which comprises a methacrylic anhydride-hyaluronic acid (HAMA); i.e., MeHA formed by reaction of methacrylic anhydride with hyaluronic acid. In certain embodiments, the chemically-modified HA comprises a methacrylated hyaluronic acid (MeHA) which comprises a glycidyl methacrylate-hyaluronic acid (HAGM); i.e., MeHA formed by reaction of glycidyl methacrylate with hyaluronic acid. In certain embodiments, methacrylation of HA can be performed by ring opening reaction of the HA backbone in combination with a reversible transesterification reaction. FIG. 1B provides an example of a reaction in which hyaluronic acid is modified with glycidyl methacrylate to form a HAGM form of methacrylated hyaluronic acid (MeHA).

In certain embodiments, a chemically-modified HA (e.g., MeHA) can be present in a polymer composition at a concentration from about 1% and about 60% weight per volume (w/v). In certain embodiments, a chemically-modified HA (e.g., MeHA) can be present in a polymer composition at a weight per volume concentration (w/v) of about 0.5%, about 1%, about 2%, about 3%, about 4%, about 5%, about 6%, about 7%, about 8% about 9%, about 10%, about 11% about 12%, about 13%, about 14%, about 15%, about 16%, about 17%, about 18% about 19%, about 20%, about 21% about 22%, about 23%, about 24%, about 25%, about 26%, about 27%, about 28% about 29%, about 30%, about 31% about 32%, about 33%, about 34%, about 35%, about 36%, about 37%, about 38% about 39%, about 40%, about 41% about 42%, about 43%, about 44%, about 45%, about 46%, about 47%, about 48% about 49%, about 50%, about 51% about 52%, about 53%, about 54%, about 55%, about 56%, about 57%, about 58% about 59%, or about 60%. In certain embodiments, a chemically-modified HA (e.g., MeHA) can be present in a polymer composition at a weight per volume concentration (w/v) of from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, about 45-50%, about 51-53%, about 53-56%, about 56-60%, about 50-60%, about 50-55%, or about 55-60%.

In certain embodiments, a polymer composition of the present disclosure comprises acryloyl-substituted gelatin (e.g., GelMA) and acryloyl-substituted hyaluronic acid (e.g., MeHA) at a ratio from about 30:1 to about 1:30 w/w. In certain embodiments, a polymer composition of the present disclosure comprises acryloyl-substituted gelatin and acryloyl-substituted hyaluronic acid in a ratio (w/w) of about 30:1, about 29:1, about 28:1, about 27:1, about 26:1, about 25:1, about 24:1, about 23:1, about 22:1, about 21:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, or about 1:30.

In certain embodiments, and acryloyl-substituted hyaluronic acid (e.g., MeHA) can be synthesized as taught in Bencherif et al., Biomaterials 29, 1739-1749 (2008); or Prata et al., Biomacromolecules 11, 769-775 (2010); each of which is incorporated herein by reference in its entirety, insofar as each describes the composition, production, analysis and use of acryloyl-substituted hyaluronic acid polymer compositions such as MeHA.

### Chemically Modified Poly(Ethylene Glycol)

Poly(ethylene glycol) (PEG) is a synthetic linear polymer which is known to have high biocompatibility and immuno-tolerability in the human body, and is soluble in many aqueous and organic solvents. In certain embodiments, polymer compositions of the present disclosure comprises a chemically-modified PEG. In certain embodiments, a polymer composition comprises acryloyl substituted PEG. In certain embodiments, a polymer composition comprises one or more acryloyl substituted PEG selected from: PEG diacrylate (PEGDA), PEG monoacrylate, PEG dimethacrylate PEG monomethacrylate, methoxy PEG acrylate, methoxy PEG methacrylate, ethoxy PEG acrylate, ethoxy PEG methacrylate, propoxy PEG acrylate, or propoxy PEG methacrylate.

In certain embodiments, a polymer compositions comprises Poly(ethylene glycol) diacrylate (PEGDA). In certain embodiments, a chemically modified PEG can be included in precursor polymer compositions of the present disclosure. In certain embodiments, the chemically-modified PEG comprises a photo-crosslinkable derivative of PEG. In certain embodiments, the chemically-modified PEG comprises Poly(ethylene glycol) diacrylate (PEGDA). In certain embodiments, chemical modification of PEG can be performed by reacting PEG with acryloyl chloride or functionally-similar acrylating compound. FIG. 1C provides an example of a reaction in which Poly(ethylene glycol) (PEG) is modified with acryloyl chloride to form Poly(ethylene glycol) diacrylate (PEGDA).

In certain embodiments, the chemically-modified PEG has a molecular weight from about 5 kDa to about 200 kDa. In certain embodiments, the chemically-modified PEG can have molecular weight from about 5-10 kDa, about 10-15 kDa, about 15-20 kDa, about 20-25 kDa, about 25-30 kDa, about 30-35 kDa, about 35-40 kDa, about 40-45 kDa, about 45-50 kDa, about 50-55 kDa, about 55-60 kDa, about 60-65 kDa, about 65-70 kDa, about 70-75 kDa, about 75-80 kDa, about 80-85 kDa, about 85-90 kDa, about 90-95 kDa, about 95-100 kDa, about 100-105 kDa, about 105-110 kDa, about 110-115 kDa, about 115-120 kDa, about 120-125 kDa, about 125-130 kDa, about 130-135 kDa, about 135-140 kDa, about 140-145 kDa, about 145-150 kDa, about 150-155 kDa, about 155-160 kDa, about 160-165 kDa, about 165-170 kDa, about 170-175 kDa, about 175-180 kDa, about 180-185 kDa, about 185-190 kDa, about 190-195 kDa, or about 195-200 kDa.

In certain embodiments, a chemically-modified PEG (e.g., PEGDA) can be present in the polymer composition at a concentration from about 1% and about 60% weight per volume (w/v). In certain embodiments, a chemically-modified PEG (e.g., PEGDA) can be present in the polymer composition at a weight per volume concentration (w/v) of about 0.5%, about 1%, about 2%, about 3%, about 4%, about 5%, about 6%, about 7%, about 8% about 9%, about 10%, about 11% about 12%, about 13%, about 14%, about 15%, about 16%, about 17%, about 18% about 19%, about 20%, about 21% about 22%, about 23%, about 24%, about 25%, about 26%, about 27%, about 28% about 29%, about 30%, about 31% about 32%, about 33%, about 34%, about 35%, about 36%, about 37%, about 38% about 39%, about 40%, about 41% about 42%, about 43%, about 44%, about 45%, about 46%, about 47%, about 48% about 49%, about 50%, about 51% about 52%, about 53%, about 54%, about 55%, about 56%, about 57%, about 58% about 59%, or about 60%. In certain embodiments, a chemically-modified PEG (e.g., PEGDA) can be present in the polymer composition at a weight per volume concentration (w/v) of from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, about 45-50%, about 51-53%, about 53-56%, about 56-60%, about 50-60%, about 50-55%, or about 55-60%.

In certain embodiments, a polymer composition of the present disclosure comprises acryloyl-substituted gelatin (e.g., GelMA) and acryloyl-substituted PEG (e.g., PEGDA) at a ratio from about 30:1 to about 1:30 w/w. In certain embodiments, a polymer composition of the present disclosure comprises acryloyl-substituted gelatin and acryloyl-substituted PEG in a ratio (w/w) of about 30:1, about 29:1, about 28:1, about 27:1, about 26:1, about 25:1, about 24:1, about 23:1, about 22:1, about 21:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, or about 1:30.

In certain embodiments, a polymer composition of the present disclosure comprises acryloyl-substituted PEG (e.g., PEGDA) and acryloyl-substituted hyaluronic acid (e.g., MeHA) at a ratio from about 30:1 to about 1:30 w/w. In certain embodiments, a polymer composition of the present disclosure comprises acryloyl-substituted PEG and acryloyl-substituted hyaluronic acid in a ratio (w/w) of about 30:1, about 29:1, about 28:1, about 27:1, about 26:1, about 25:1, about 24:1, about 23:1, about 22:1, about 21:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, or about 1:30.

In certain embodiments, a polymer composition of the present disclosure comprises one or more synthetic polymer components (i.e., polymer or precursors) selected from methacrylate-oligolactide-PEO-oligolactide-methacrylate, Polyethylene glycol (PEG), polyglycerol sebacate(PGS), polylactic acid (PLA), polypropylene glycol (PPO), PEG-PPO-PEG copolymers (e.g., pluronics), polyphosphazene, polymethacrylates, poly(N-vinylpyrrolidone), and polyethyleneimine.

### Crosslinking Agents

In certain embodiments, a polymer composition of the present disclosure comprises a crosslinking agent. As used herein, the phrase “crosslinking agent” can describe a substance which forms, promotes, or regulates intermolecular bonding (covalent, ionic, hydrogen) between polymeric units or chains to create a network of polymeric chains. Crosslinking agents typically exhibit one or more, optionally two or more, bonding functionalities which can create chemical bonds between two or more polymer chains. Crosslinking agents can include, for example, two vinyl bonds (tetrafunctionality), or three amines (trifunctionality).

In certain embodiments, a polymer composition comprises a crosslinking agent which can be used to activate or facilitate polymerization, gelation, and solidification of the polymer composition from a precursor polymer composition to a gel polymer composition. In certain embodiments, exposure of a polymer composition of the present disclosure (e.g., precursor polymer composition) to crosslinking conditions (e.g. exposure to visible light in the presence of a photoinitiator) can result in one or more acryloyl groups in the polymer composition (e.g., acryloyl-substituted gelatin, acryloyl-substituted HA, acryloyl substituted PEG, and other acryloyl-based crosslinking agents) to react with other acryloyl groups to crosslink the polymer composition and form a gel polymer composition (e.g., GelMA hydrogel).

In certain embodiments, a polymer composition of the present disclosure (e.g., precursor polymer composition) comprises from about 1% and about 50% (w/v) of one or more crosslinking agents. In certain embodiments, the polymer composition comprises one or more crosslinking agents at a concentration (w/v) of at least about 5%, about 10%, about 15%, about 20%, about 25%, about 30%, about 35%, or about 40%. In certain embodiments, the polymer composition comprises one or more crosslinking agents at a concentration (w/v) of no more than about 50%, about 45%, about 40%, about 35%, or about 30%. In certain embodiments, the polymer composition comprises one or more crosslinking agents at a concentration (w/v) of from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, or about 45-50%.

In certain embodiments, a polymer composition of the present disclosure comprises one or more crosslinking agents selected from glutaraldehyde, epoxides (e.g., bis-oxiranes), oxidized dextran, p-azidobenzoyl hydrazide, N-(a-maleimidoacetoxy)succinimide ester, p-azidophenyl glyoxal monohydrate, bis-((4-azidosalicylamido)ethyl)disulfide, bis(sulfosuccinimidyl)suberate, dithiobis(succinimidyl proprionate), disuccinimidyl suberate, 1-ethyl-3-(3-dimethylaminopropyl)carbodiimide hydrochloride (EDC), ethoxylated trimethylpropane triacrylate, N-hydroxysuccinimide (NHS), derivatives thereof, or a combination thereof.

In certain embodiments, a polymer composition of the present disclosure comprises one or more crosslinking agents selected from polyethyleneoxide dimethacrylate, methylene bisacrylamide, methylene bis(2-methylacrylamide), methylene diacrylate, methylene bis(2-methylacrylate), diethylene glycol diacrylate, hexamethylene diacrylate, hexamethylene diisocyanate, oxybis(methylene) bis(2-methylacrylate), oxybis(ethane-2,1-diyl) bis(2-methylacrylate), trimethylolpropane triacrylate, pentaerythritol triacrylate, tris (2-hydroxy ethyl) isocyanurate triacrylate, isocyanuric acid tris(2-acryloyloxyethyl) ester, ethoxylated trimethylolpropane triacrylate, pentaerythrityl triacrylate and glycerol triacrylate, phosphinylidynetris(oxyethylene) triacrylatederivatives thereof, or a combination thereof

### Polymer Crosslinking Initiator/Photo-Initiator

In certain embodiments, a polymer composition comprises one or more polymer crosslinking initiators, such as photo-initiator elements. In certain embodiments, the polymer crosslinking initiator forms free-radicals when exposed to specific polymer crosslinking conditions (e.g., acidic conditions, basic conditions, high-salt conditions, low salt conditions, high temperature, agitation, solubility conditions, light exposure), wherein the free radicals can result in bond formation between reactive groups in the composition, such as vinyl-bond crosslinking between methacrylate groups in a GelMA polymer composition.

In certain embodiments, a polymer composition comprises one or more photo-initiator elements (i.e., a crosslinking initiator which is initiated or activated by absorbing a certain wavelength of light). In certain embodiments, precursor polymer compositions of the present disclosure comprises one or more photo-initiator elements. In certain embodiments, the photo-initiator element can be activated by exposure to light. In certain embodiments, light exposure can activate the photo-initiator to form free-radicals, wherein the free radicals can result in bond formation between reactive groups in the composition, such as vinyl-bond crosslinking between methacrylate groups in a GelMA polymer composition.

In certain embodiments, a photo-initiator element can be activated by exposure to one or more light sources selected from visible light sources (e.g., white or blue light), ultraviolet (UV) light sources, near-infrared (NIR) light sources, and fluorescent light sources. In certain embodiments, the photo-initiator element comprises a visible light-activated photo-initiator, such as a visible light-activated photo-initiator which is activated upon exposure to light having a wavelength from about 380 nm to about 740 nm. In certain embodiments, the visible light-activated photo-initiator can be activated upon exposure to light having a wavelength of from about 380-435 nm (i.e. violet light), about 435-500 nm (i.e. blue light), about 500-565 nm (i.e. green light), about 565-600 nm (i.e. yellow light), about 600-650 nm (i.e. orange light), or about 650-740 nm (i.e. red light). In certain embodiments, the photo-initiator element comprises an ultraviolet light-activated photo-initiator. In certain embodiments, the photo-initiator element comprises a near-infrared (NIR) light-activated photo-initiator. In certain embodiments, the photo-initiator element comprises a white light-activated photo-initiator. In certain embodiments, the photo-initiator element comprises a blue light-activated photo-initiator.

In certain embodiments, a polymer composition comprises one or more photo-initiator elements selected from: triethanolamine; 1-Vinyl-2-pyrrolidone (NVP); N-vinylcaprolactam (NVC); Ethylene Glycol Diacrylate (EGDA); riboflavin; azobisisobutyronitrile; benzoyl peroxide; 1-benzoylcyclohexanol; di-tert-butyl peroxide; Eosin Y (e.g., disodium salt), (2-(2,4,5,7-tetrabromo-6-oxido-3-oxo-3H-xanthen-9-yl) benzoate); 4,6-trimethylbenzoylphosphinate; triethanol amine; 2,3-diketo-1,7,7-trimethylnorcamphane; 1-phenyl-1,2-propadione; 2,4,6-trimethylbenzoyl-diphenylphosphine oxide; bis(2,6-dichlorobenzoyl)-(4-propylphenyl)phosphine oxide; 4,4′-bis(dimethylamino)benzophenone; 4,4′-bis(diethylamino)benzophenone; 2-chlorothioxanthen-9-one; 4-(dimethylamino)benzophenone; phenanthrenequinone; ferrocene; 2-hydroxy-4′-(2-hydroxyethoxy)-2-methylpropiophenone; 2-hydroxy-2-methylpropiophenone; diphenyl(2,4,6 trimethylbenzoyl)phosphine oxide/2-hydroxy-2-methylpropiophenone (blend); benzoin methyl ether; benzoin isopropyl ether; 2,2-diethoxyacetophenone; dibutoxyacetophenone; 2,2-dimethoxy-2-phenyl-1-phenylethanone; 2,2-dimethoxy-2-phenylacetophenone; dibenzosuberenone; (benzene)tricarbonylchromium; resazurin; resorufin; benzoyltrimethylgermane; lithium phenyl-2,4,6-trimethyl-benzoylphospinate; camphorquinone; 2-methyl-1-(4-(methylthio)phenyl)-2-morpholinopropan-2-one; 2-benzyl-2-(dimethylamino)-4′-morpholinobutyrophenone; 2-benzyl-2-dimethylamino-1-(4-morpholinophenyl)butan-1-one; methybenzoylformate; bis(2,4,6-trimethylbenzoyl)-phenylphosphineoxide; bis(2,4-cylcopentadien-1-yl)-bis(2,6-difluoro-3-(1H-pyrrol-1-yl)-phenyl)titanium; 5,7-diiodo-3-butoxy-6-fluorone; 2,4,5,7-tetraiodo-3-hydroxy-6-fluorone; 2,4,5,7-tetraiodo-3-hydroxy-9-cyano-6-fluorone; dimethoxyhydroxy-acetophenone; 2-naphthalene-sulfonyl chloride; 1-phenyl-1,2-propanedione-2-(O-ethoxy-carbonyl)oxime; 2-ethylthioxanthone; 2-isopropylthioxanthone; 2,4-diethyl thioxanthone; 2-tert-butyl thioxanthone; 2-chlorothioxanthone; 2-propoxy thioxanthone; methylphenylglycoxylate; phenyl 2-hydroxy-2-propyl ketone; 4-isopropylphenyl 2-hydroxy-2-propyl ketone; 4-n-dodecylphenyl 2-hydroxy-2propyl ketone; 4-(2-hydroxyethoxy)phenyl 2-hydroxy-2-propyl ketone; 1-[4-(2-hydroxyethoxy)-phenyl]-2-hydroxy-2-methyl-1-propane-1-one); 4-(2-acryloyloxyethoxy)phenyl 2-hydroxy-2-propyl ketone; derivatives thereof, or combinations thereof. In certain embodiments, a polymer composition comprises a combination of Eosin Y, triethanolamine, and/or vinyl caprolactam. In certain embodiments, the polymer crosslinking initiator comprises eosin Y disodium salt (EYDS), N-vinylcaprolactam (NVC), triethanolamine, or any combination thereof. In certain embodiments, the polymer crosslinking initiator comprises eosin Y disodium salt (EYDS), N-Vinylpyrrolidone (NVP), triethanolamine, or any combination thereof.

In certain embodiments, the polymer crosslinking initiator comprises: (i) about 50 μM eosin Y or eosin Y disodium salt (EYDS), optionally about 50 M eosin Y disodium salt (EYDS); (ii) from about 3.5 to about 5.0 μL/mL of N-vinylcaprolactam (NVC) or N-Vinylpyrrolidone (NVP), optionally from about 3.5 to about 5.0 μL/mL of NVP, optionally about 5.0 μL/mL of NVP; and (iii) triethanolamine, optionally about 1.5% v/v of triethanolamine.

In certain embodiments, a polymer composition comprises one or more photo-initiator elements selected from: acetophenone; anisoin; anthraquinone; anthraquinone-2-sulfonic acid, sodium salt monohydrate; (benzene) tricarbonylchromium; 4-(boc-aminomethyl)phenyl isothiocyanate; benzin; benzoin; benzoin ethyl ether; benzoin isobutyl ether; benzoin methyl ether; benzoic acid; benzophenyl-hydroxycyclohexyl phenyl ketone; 3,3′,4,4′-benzophenone tetracarboxylic dianhydride; 4-benzoylbiphenyl; 2-benzyl-2-(dimethylamino)-4′-morpholino butyrophenone; 4,4′-bis(diefhylamino)benzophenone; 4,4′-bis(dimethylamino)benzophenone; Michler's ketone; camphorquinone; 2-chlorothioxanthen-9-one; 5-dibenzosuberenone; (cumene)cyclopentadienyliron(II) hexafluorophosphate; dibenzosuberenone; 2,2-diefhoxyacetophenone; 4,4′-dihydroxybenzophenone; 2,2-dimethoxy2-phenylacetophenone; 4-(dimethylamino)benzophenone; 4,4′-dimethylbenzyl; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenvldithiocarbonate; 2,2′-azobisisobutyronitrile; 2,2′-azobis[2-methyl-N-(2-hydroxyethyl)propionamide; camphorquinone; eosin; dimethylaminobenzoate; dimethoxy-2-phenyl-acetophenone; Quanta-cure ITX photosensitizer; Irgacures (e.g., 907, 2959, 651); Darocur 2959; ethyl-4-N,N-dimethylaminobenzoate; 1-[-(4-benzoylphenylsulfanyl)phenyl]-2-methyl-2-(4-methylphenylsulfonyl)propan-1-one; 1-hydroxy-cyclohexyl-phenyl-ketone; 2,4,6-trimethylbenzoyldiphenylphosphine oxide; diphenyl(2,4,6-trimethylbenzoyl)phosphine; 2-ethylhexyl-4-dimethylaminobenzoate; 2-hydroxy-2-methyl-1-phenyl-1-propanone; oligo[2-hydroxy-2-methyl-1-[4-(methylvinyl)phenyl]propanone] and propoxylated glyceryl triacrylate; benzii dimethyl ketal; benzophenone; blend of benzophenone and α-hydroxy-cyclohexyl-phenylketone; blend of Esacure KIP 150 and Esacure TZT; blend of Esacure KIP150 and Esacure TZT; blend of Esacure KIP150 and TPGDA; blend of phosphine oxide. Esacure KIP 150 and Esacure TZT; difunctional α-hydroxy ketone; ethyl 4-dimethylaminobenzoate; isopropyl thioxanthone; 2-hydroxy-2methyl-phenylpropanone; 2,4,6,-trimethylbenzoyldiphenyl phosphine oxide; 2,4,6-trimethyl benzophenone; blend of 4-methylbenzophenone and benzophenone; oligo(2-hydroxy-2-methyl-1-(4(1-methylvinyl)phenyl)propanone; oligo(2-hydroxy-2-methyl-1-4(1-methylvinyl)phenyl propanone and 2-hydroxy-2-methyl-1-phenyl-1-propanone; 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone.

In certain embodiments, a polymer composition comprises one or more cationic and/or anionic photo-initiator elements selected from: titanium tetrachloride, vanadium tetrachloride, bis(cyclopentadienyl)titanium dichloride, ferrocene, cyclopentadienyl manganese tricarbonyl, manganese decacarbonyl, diazonium salts, diaryliodonium salts (e.g., 3,3′-dinitrodiphenyliodonium hexafluoroarsenate, diphenyliodonium fluoroborate, 4-methoxydiphenyliodonium fluoroborate) and triarylsulfonium salts.

Photoinitiated polymerizations and photoinitiators are discussed in detail in Rabek, Mechanisms of Photophysical Processes and Photochemical Reactions in Polymers, New York: Wiley & Sons, 1987; and Fouassier, Photoinitiation, Photopolymerization, and Photocuring, Cincinnati, Ohio: Hanser/Gardner; Fisher etal., 2001, Annu. Rev. Mater. Res., 31:171; each of which is incorporated herein by reference in its entirety, insofar as each describes the use of polymerization and photoinitiators in the production of polymer compositions, including acryloyl gelatins such as GelMA hydrogels.

In certain embodiments, a polymer composition comprises a crosslinking agent or initiator which comprises one or more metal²⁺ ions and/or metal³⁺ ions. In certain embodiments, a polymer composition comprises a crosslinking agent which comprises one or more metal²⁺ ions and/or metal³⁺ ions selected from Fe²⁺, Fe³⁺, Ni²⁺, Zn²⁺, Cu²⁺, Ag²⁺, Au³⁺, Co²⁺, Co³⁺, Cr²⁺, Cr³⁺, Cd²⁺, Mn²⁺, Mg²⁺, Pd²⁺, Pt²⁺, Al³⁺, or combinations thereof. In certain embodiments, a precursor polymer composition of the present disclosure comprises both one or more photoinitiators element and one or more metal^{2+/3+} ions.

In certain embodiments, a polymer composition comprises a crosslinking agent or initiator which uses Click bioconjugation chemistry for polymeric crosslinking. In certain embodiments, the polymer composition comprises a crosslinking agent or initiator which uses Click bioconjugation chemistry selected from metal-catalyzed azide-alkyne cycloaddition, strain-promoted azide-alkyne cycloaddition, strain-promoted alkyne-nitrone cycloaddition (e.g., Alkene/azide [3+2]cycloaddition, Alkene/tetrazine inverse-demand Diels-Alder, Alkene/tetrazole photoclick reaction), or a combination thereof.

### II. Physical, Mechanical and Structural Characteristics

### Viscosity, Shear Strength and Shear Resistance

The viscosity of a material is a measurement of the resistance of the material to deformation at a given rate. The viscosity of a fluid material is often correlated with the thickness and/or density of that material.

In certain embodiments, polymer compositions of the present disclosure can have a therapeutically-effective viscosity. In certain embodiments, a polymer composition can have a viscosity which provides for strong adhesion and high retention of the polymer composition on a target tissue of a subject. In certain embodiments, a precursor polymer composition of the present disclosure can have a viscosity which provides for strong adhesion and high retention of the polymer composition on a target tissue of a subject. In certain embodiments, a precursor polymer composition can have a viscosity which is greater than water. In certain embodiments, a precursor polymer composition can have a viscosity which is equivalent to a paste. In certain embodiments, a gel polymer composition of the present disclosure can have a viscosity which provides for strong adhesion and high retention of the polymer composition on a target tissue of a subject. In certain embodiments, a precursor polymer composition can have a viscosity which is equivalent to water. In certain embodiments, a gel polymer composition can retain its shape and/or consistency on the surface of a target tissue for one or more hours, one or more days, or one or more weeks.

In certain embodiments, a polymer composition can have a viscosity from about 0.5 Pascal-seconds (Pa·s) to about 300 Pa·s at a low shear rate (e.g., at a shear rate of about 0.001 s⁻¹ to about 1 s⁻¹). In certain embodiments, the polymer composition can have a viscosity from about 0.5-100 Pas at a low shear rate. In certain embodiments, the polymer composition can have a viscosity, at a low shear rate, of from about 0.5-5 Pa·s, about 5-10 Pa·s, about 10-15 Pa·s, about 15-20 Pa·s, about 20-25 Pa·s, about 25-30 Pa·s, about 30-35 Pa·s, about 35-40 Pa·s, about 40-45 Pa·s, about 45-50 Pa·s, about 50-55 Pa·s, about 55-60 Pa·s, about 60-65 Pa·s, about 65-70 Pa·s, about 70-75 Pa·s, about 75-80 Pa·s, about 80-85 Pa·s, about 85-90 Pa·s, about 90-95 Pa·s, about 95-100 Pa·s, about 100-125 Pa·s, about 125-150 Pa·s, about 150-175 Pa·s, about 175-200 Pa·s, about 200-225 Pa·s, about 225-250 Pa·s, about 250-275 Pa·s, or about 275-300 Pa·s.

Shear strength and/or resistance are measurements of the ability of a material to resist external shear stress (i.e., shear load) without failure (i.e. loss of adhesion or integrity). In certain embodiments, polymer compositions of the present disclosure can have a therapeutically-effective shear strength. In certain embodiments, a polymer composition can have a shear strength which provides for durable adhesion and high retention of the polymer composition on a target tissue of a subject. In certain embodiments, a gel polymer composition of the present disclosure can have a shear strength which provides for durable adhesion and high retention of the polymer composition on a target tissue of a subject. In certain embodiments, a gel polymer composition can have a shear strength which allows the polymer composition to retain its shape, adhesion, connectivity and/or consistency on the surface of a target tissue for one or more hours, one or more days, or one or more weeks.

In certain embodiments, a polymer composition can have shear strength from about 1 to about 360 kPa. In certain embodiments, the polymer composition can have shear strength from about 100-360 kPa. In certain embodiments, the polymer composition can have shear strength from about 200-360 kPa. In certain embodiments, the polymer composition can have a shear strength from about 1-20 kPa, about 20-40 kPa, about 40-60 kPa, about 60-80 kPa, about 80-100 kPa, 100-120 kPa, about 120-140 kPa, about 140-160 kPa, about 160-180 kPa, about 180-200 kPa, 200-220 kPa, about 220-240 kPa, about 240-260 kPa, about 260-280 kPa, about 280-300 kPa, 300-320 kPa, about 320-340 kPa, or about 340-360 kPa.

In certain embodiments, the shear strength of a polymer composition can be measured using ASTM F2255-05, or a modified Lap Shear test variation thereof.

### Swelling and Water Content

In certain embodiments, the polymer composition comprises a gel. A gel generally comprises a crosslinked polymeric framework which encompasses a network of pores filled with an interstitial solvent (e.g., a fluid). In certain embodiments, the polymer composition comprises a hydrogel, wherein the interstitial fluid comprises water. In certain embodiments, the polymer composition comprises an alcogel, wherein the interstitial fluid comprises an alcohol (e.g., methanol, ethanol).

Swelling (i.e., an increase in volume) can occur in a gel when the gel material absorbs and retains additional interstitial fluid within the pore network of the gel. Likewise, shrinkage (i.e., a decrease in volume) can occur in a gel when the gel material expels interstitial fluid from the pore network of the gel. The ability and/or tendency of a gel material to swell and/or shrink in certain solvent environments will depend on the chemical nature of the polymer and the solvent (e.g., solubility, hydrophobicity, pore structure, affinity) and the elasticity of the polymer network of the gel. The swelling ratio of a gel is a measurement of the fractional increase in the weight of the gel due to fluid absorption (e.g., weight increase of a hydrogel from the absorption of water).

In certain embodiments, polymer compositions of the present disclosure can have a therapeutically-effective swelling ratio and/or water content. In certain embodiments, a polymer composition can have a swelling ratio and/or water content which provides for strong adhesion and high retention of the polymer composition on a target tissue of a subject. In certain embodiments, a gel polymer composition of the present disclosure can have a swelling ratio and/or water content which provides for strong adhesion and high retention of the polymer composition on a target tissue of a subject. In certain embodiments, a gel polymer composition can have a swelling ratio and/or water content which allows the polymer composition to retain its shape, adhesion, connectivity and/or consistency on the surface of a target tissue for one or more hours, one or more days, or one or more weeks.

In certain embodiments, a polymer composition can have a swelling ratio from about 5% to about 50%. In certain embodiments, a polymer composition can have a swelling ratio of at least about 5%, about 10%, about 15%, about 20%, about 25%, about 30%, about 35%, or about 40%. In certain embodiments, a polymer composition can have a swelling ratio of no more than about 50%, about 45%, about 40%, about 35%, about 30%, about 25%, about 20%, about 15%, or about 10%. In certain embodiments, a polymer composition has a swelling ratio of about 25% or less, about 20% or less, about 15% or less, or about 10% or less. In certain embodiments, a polymer composition can have a swelling ratio from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, or about 45-50%. In certain embodiments, a polymer composition can have a short-term swelling ratio (i.e., a swelling ratio measured for about 1 to 24 hours) from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, or about 45-50%. In certain embodiments, a polymer composition can have a medium-term swelling ratio (i.e., a swelling ratio measured for about 1 to 7 days) from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, or about 45-50%. In certain embodiments, a polymer composition can have a long-term swelling ratio (i.e., a swelling ratio measured for about 1 to 4 weeks, or more) from about 1-3%, about 3-6%, about 6-10%, about 1-5%, about 1-10%, about 5-10%, about 11-13%, about 13-16%, about 16-20%, about 10-20%, about 10-15%, about 15-20%, about 21-23%, about 23-26%, about 26-30%, about 20-30%, about 20-25% about 25-30%, about 31-33%, about 33-36%, about 36-40%, about 30-40%, about 30-35% about 35-40%, about 41-43%, about 43-46%, about 46-50%, about 40-50%, about 40-45%, or about 45-50%.

In certain embodiments, a hydrogel polymer composition can have a water content from about 5% to about 99%. In certain embodiments, a hydrogel polymer composition can have a water content from about 50% to about 99%. In certain embodiments, a hydrogel polymer composition can have a water content from about 65% to about 85%. In certain embodiments, a polymer composition can have a water content of at least about 5%, about 10%, about 15%, about 20%, about 25%, about 30%, about 35%, about 40%, about 45%, about 50%, about 55%, about 60%, about 65%, about 70%, about 75%, or about 80%. In certain embodiments, a polymer composition can have a swelling ratio of about 99% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, or about 30% or less. In certain embodiments, a polymer composition can have a water content from about 1-3%, about 3-6%, about 6-10%, about 1-5%, about 5-10%, about 1-10%, about 11-13%, about 13-16%, about 16-20%, about 10-15%, about 15-20%, about 10-20%, about 21-23%, about 23-26%, about 26-30%, about 20-25%, about 25-30%, about 20-30%, about 31-33%, about 33-36%, about 36-40%, about 30-35% about 35-40%, about 30-40%, about 41-43%, about 43-46%, about 46-50%, about 40-45%, about 45-50%, about 40-50%, about 51-53%, about 53-56%, about 56-60%, about 50-55%, about 55-60%, about 50-60%, about 61-63%, about 63-66%, about 66-70%, about 60-65%, about 65-70%, about 60-70%, about 71-73%, about 73-76%, about 76-80%, about 70-75%, about 75-80%, about 70-80%, about 81-83%, about 83-86%, about 86-90%, about 80-85% about 85-90%, about 80-90%, about 91-93%, about 93-96%, about 96-99%, about 90-95%, about 95-99%, or about 90-99%.

In certain embodiments, a hydrogel polymer composition of the present disclosure permits controlled and sustained release of one or more therapeutic agents over a period of time. In certain embodiments, the hydrogel polymer composition allows for the release of at least 1 μg/day, at least 2 μg/day, at least 3 μg/day, at least 4 μg/day, at least 5 μg/day, at least 6 μg/day, at least 7 μg/day, at least 8 μg/day, at least 9 μg/day, at least 10 μg/day, at least 11 μg/day, at least 12 μg/day, at least 13 μg/day, at least 14 μg/day, at least 15 μg/day, at least 16 μg/day, at least 17 μg/day, at least 18 μg/day, at least 19 μg/day, at least 20 μg/day, at least 25 μg/day, at least 30 μg/day, at least 35 μg/day, at least 40 μg/day, at least 45 μg/day, at least 50 μg/day, at least 60 μg/day, at least 70 μg/day, at least 80 μg/day, at least 90 μg/day, at least 100 μg/day, at least 150 μg/day, at least 200 μg/day, at least 250 μg/day, at least 300 μg/day, at least 350 μg/day, at least 400 μg/day, at least 450 μg/day, at least 500 μg/day, at least 600 μg/day, at least 700 μg/day, at least 800 μg/day, at least 900 μg/day, or at least 1000 μg/day of a therapeutic agent. In certain embodiments, the hydrogel polymer composition allows for the release of at least 10 μg/day of a therapeutic agent.

### Durability and Degradation

In certain embodiments, polymer compositions of the present disclosure can have a therapeutically-effective rate of polymeric degradation (i.e. degradation rate). In certain embodiments, a polymer composition can have a degradation rate which provides for sustained adhesion and high retention of the polymer composition on a target tissue of a subject. In certain embodiments, a gel polymer composition of the present disclosure can have a degradation rate which provides for sustained adhesion and high retention of the polymer composition on a target tissue of a subject. In certain embodiments, a gel polymer composition can have a degradation rate which allows the polymer composition to retain its shape, adhesion, connectivity and/or consistency on the surface of a target tissue for one or more hours, one or more days, or one or more weeks.

In certain embodiments, a polymer composition can have a degradation rate from 1-50 days. In certain embodiments, a polymer composition can have a degradation rate from about 1-3 days, about 3-6 days, about 6-10 days, about 1-5 days, about 1-10 days, about 5-10 days, about 11-13 days, about 13-16 days, about 16-20 days, about 10-20 days, about 10-15 days, about 15-20 days, about 21-23 days, about 23-26 days, about 26-30 days, about 20-30 days, about 20-25 days about 25-30 days, about 31-33 days, about 33-36 days, about 36-40 days, about 30-40 days, about 30-35 days about 35-40 days, about 41-43 days, about 43-46 days, about 46-50 days, about 40-50 days, about 40-45 days, or about 45-50 days.

### Biocompatibility

In certain embodiments, the polymer compositions of the present disclosure have biocompatibility with a target tissue of a subject. In certain embodiments, the biomechanical properties of the polymer compositions are similar and/or biocompatible to the biomechanical properties of a target tissue of a subject (e.g., the cornea of a subject).

In certain embodiments, the biocompatibility of a polymer compositions can be evidenced by low inflammatory response in a target tissue or subject. In certain embodiments, the biocompatibility of a polymer compositions can be evidenced by the survival rate of cells from a target tissue which are implanted or incorporated into a portion of the polymer composition.

### Shape

In certain embodiments, polymer compositions of the present disclosure can be formed as molded, stamped, or shaped gel compositions. Molded, stamped or shaped hydrogels can be prepared using, for example, the methods set forth in US 20050008675 or US 20040258729, each of which is incorporated herein by reference in its entirety, insofar as each describes the composition, production (including molding), analysis and use of hydrogels, including acrylated gelatin polymeric compositions such as GelMA hydrogels.

In certain embodiments, polymer compositions (e.g., hydrogel polymer compositions) of the present disclosure can be formed into cylinders, each cylinder having a length and a diameter. In certain embodiments, polymer compositions (e.g., hydrogel polymer compositions) of the present disclosure can be conformed to the shape of the target surface. In certain embodiments, the polymer composition is conformed to the convex, concave, or curved shape of a target surface.

In certain embodiments, polymer compositions can be formed into cylindrical rods. As used herein, “cylindrical rods” or “rods” describe cylinders which have a cylinder-length at least 3-times (3×) the cylinder-diameter. As not limiting examples, a cylindrical rod can have: a length of about 3 mm and a diameter of about 0.75 mm; or a length of about 2.5 mm and a diameter of about 0.75 mm. In certain embodiments, hydrogel rods of the present disclosure can be about 3 mm in length and about 0.75 mm in diameter. In certain embodiments, hydrogel rods of the present disclosure can be about 6 mm in length and about 0.75 mm in diameter.

In certain embodiments, polymer compositions can be formed into cylindrical disks. As used herein, “cylindrical disks” or “disks” describe cylinders which have a cylinder-diameter at least 2-times (2×) the cylinder-length. As not limiting examples, a cylindrical disk can have: a length of about 2.5 mm and a diameter of about 6 mm; or a length of about 2 nm and a diameter of about 6 mm.

### III. Gel Production

In certain embodiments, polymeric compositions of the present disclosure (e.g., GelMA polymer compositions) can be produced as described in the art, including Nichol et al., Biomaterials, 2010 Jul., 31(21):5536-44; Assmann et al., Biomaterials, 2017, 140:115-127; Noshadi et al., Biomater. Sci., 2017, 5: 2093-2105; each of which is incorporated herein by reference in its entirety, insofar as each describes the production of polymeric compositions, including acryloyl gelatin polymeric compositions such as GelMA hydrogels.

In certain embodiments, a polymer composition of the present disclosure can be formed by crosslinking two or more chemically modified gelatin components in a precursor polymer composition to form a gel polymer composition. In certain embodiments, a polymer composition of the present disclosure can crosslink, polymerize and/or gel under wet, aqueous and/or biological conditions to form a gel polymer composition. In certain embodiments, the crosslinking of the two or more chemically modified gelatin components is initiated, facilitated, or enabled when exposed to specific crosslinking conditions (e.g., acidic conditions, basic conditions, high-salt conditions, low salt conditions, high temperature, agitation, solubility conditions). In certain embodiments, the crosslinking of the two or more chemically modified gelatin components is initiated, facilitated, or enabled by a crosslinking agent. In certain embodiments, the crosslinking of the two or more chemically modified gelatin components is initiated, facilitated, or enabled by a crosslinking agent under specific crosslinking conditions.

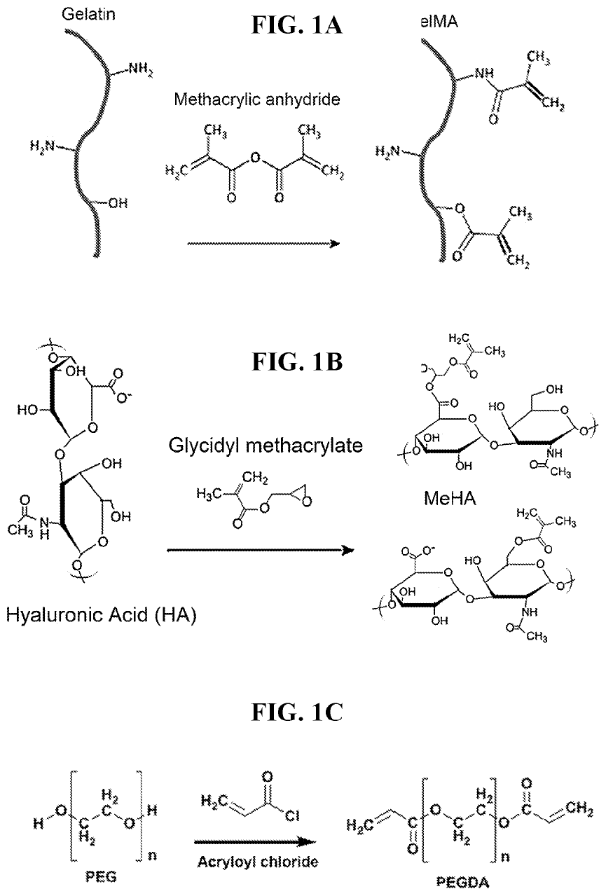

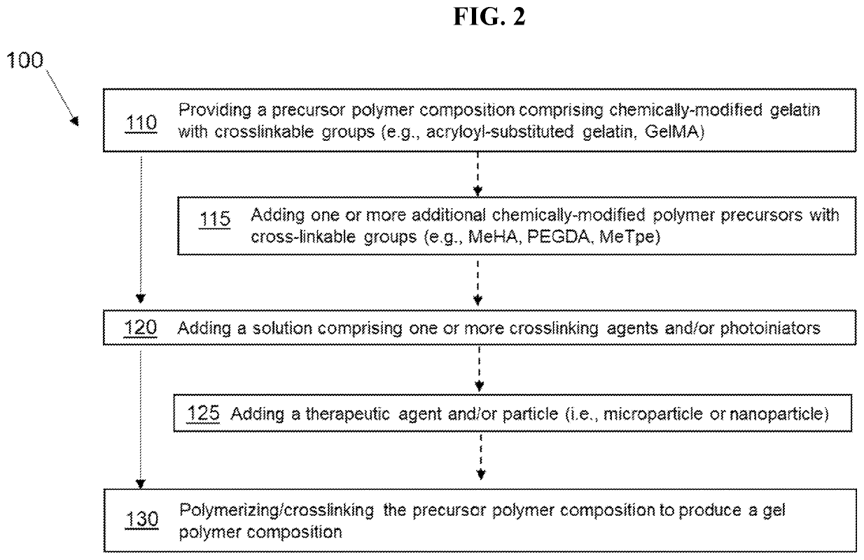

In certain embodiments, the present disclosure provides methods for producing a gel polymer composition, such as a hydrogel polymer composition. In certain embodiments, the present disclosure provides methods for producing a GelMA hydrogel polymer composition. FIG. 2 provides a method 100 for producing a gel polymer composition. In step 110, a precursor polymer composition comprising chemically-modified gelatin with crosslinkable groups (e.g., acryloyl-substituted gelatin, GelMA) is provided. In optional step 115, one or more additional chemically-modified polymer precursors with cross-linkable groups (e.g., MeHA, PEGDA) is added to the precursor polymer composition. In certain embodiments, the polymer composition comprises an unmodified HA and/or an unmodified PEG and/or an unmodified tropoelastin. In step 120, a solution comprising one or more crosslinking agents and/or photoiniators is added to the precursor polymer composition. In optional step 125, a therapeutic agent, cell, and/or particle (i.e., microparticle or nanoparticle) is added to the precursor polymer composition. In step 130, the precursor polymer composition is polymerized/crosslinked to produce a gel polymer composition.

In certain embodiments, methods for producing a gel polymer composition can include providing a precursor polymer composition comprising chemically-modified gelatin with crosslinkable groups (e.g., acryloyl-substituted gelatin, GelMA). In certain embodiments, the chemically-modified gelatin comprises acrylated gelatin. In certain embodiments, the chemically-modified gelatin comprises gelatin methacryloyl (i.e. GelMA).

In certain embodiments, the precursor polymeric composition comprises one or more solvents or liquid vehicles, diluents, dispersion media, dispersing agents, granulating agents, binding agents, disintegrating agents, suspension agents, surface active agents, emulsiflers or emulsifying agents, isotonic agents, thickening agents, preservatives, solid binders, buffering agents, lubricants, coloring agents, coating agents, sweeteners, flavourings, perfuming agents, or combinations thereof.

In certain embodiments, the precursor polymeric composition comprises one or more solvents. In certain embodiments, the solvent comprises an aqueous solvent. Examples of aqueous solvents include, but are not limited to, distilled water, deionized water, saline, Dulbecco's phosphate-buffered saline (DPBS), and Ringer's solution. In certain embodiments, the solvent comprises DPBS. In certain embodiments, the solvent comprises an organic solvent. Examples of organic solvents include, but are not limited to, hexanes, benzene, toluene, acetone, diethyl ether, chloroform, dichloromethane, isopropanol, methanol, ethanol, n-propanol, and n-butanol.

In certain embodiments, a precursor polymer composition can be in a sprayable form. In certain embodiments, a precursor polymer composition can be in a high-viscosity form (e.g., paste-like viscosity). In certain embodiments, a precursor polymer composition can be in a low-viscosity form (e.g., liquid-like viscosity).

In certain embodiments, methods for producing a gel polymer composition can include a step of adding one or more additional chemically-modified polymer precursors with cross-linkable groups to the precursor polymer composition. In certain embodiments, methods for producing a gel polymer composition can include: (i) providing a precursor polymer composition comprising chemically-modified gelatin with crosslinkable groups (e.g., acryloyl-substituted gelatin, GelMA); and (ii) adding one or more additional chemically-modified polymer precursors with cross-linkable groups to the precursor polymer composition. In certain embodiments, the one or more additional chemically-modified polymer precursors comprises a chemically-modified hyaluronic acid, such as an acryloyl-substituted hyaluronic acid. In certain embodiments, the chemically-modified hyaluronic acid comprises methacrylated hyaluronic acid (MeHA). In certain embodiments, the one or more additional chemically-modified polymer precursors comprises a chemically-modified Poly(ethylene glycol) (PEG), such as an acryloyl-substituted PEG. In certain embodiments, the chemically-modified hyaluronic acid comprises Poly(ethylene glycol) diacrylate (PEGDA).

In certain embodiments, methods for producing a gel polymer composition can include a step of adding one or more crosslinking agents and/or polymer crosslinking initiators (e.g., photoiniators) to the precursor polymer composition. In certain embodiments, methods for producing a gel polymer composition can include: (i) providing a precursor polymer composition comprising chemically-modified gelatin with crosslinkable groups (e.g., acryloyl-substituted gelatin, GelMA); and (ii) adding one or more crosslinking agents and/or polymer crosslinking initiators (e.g., photoiniators) to the precursor polymer, composition. In certain embodiments, methods for producing a gel polymer composition can include: (i) providing a precursor polymer composition comprising chemically-modified gelatin with crosslinkable groups (e.g., acryloyl-substituted gelatin, GelMA); (ii) adding one or more additional chemically-modified polymer precursors with cross-linkable groups to the precursor polymer composition; and (iii) adding one or more crosslinking agents and/or polymer crosslinking initiators (e.g., photoinitiators) to the precursor polymer.

In certain embodiments, one or more crosslinking agents and/or polymer crosslinking initiators (e.g., photoinitiators) can be added to the precursor polymer before one or more additional chemically-modified polymer precursors with cross-linkable groups are added to the precursor polymer composition. In certain embodiments, methods for producing a gel polymer composition can include: (i) providing a precursor polymer composition comprising chemically-modified gelatin with crosslinkable groups (e.g., acryloyl-substituted gelatin, GelMA); (ii) adding one or more crosslinking agents and/or polymer crosslinking initiators (e.g., photoinitiators) to the precursor polymer; and (iii) adding one or more additional chemically-modified polymer precursors with cross-linkable groups to the precursor polymer composition.

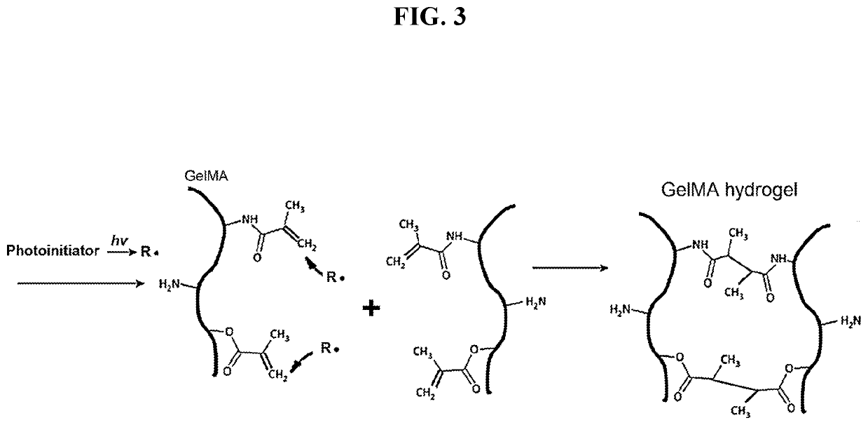

In certain embodiments, a polymer composition comprises one or more polymer crosslinking initiators, (e.g., crosslinking initiator which forms free-radicals when exposed to specific polymer crosslinking conditions, such as acidic conditions, basic conditions, high-salt conditions, low salt conditions, high temperature, agitation, solubility conditions, and light exposure). In certain embodiments, a polymer composition comprises one or more photo-initiator elements (i.e., a crosslinking initiator which is initiated or activated by absorbing a certain wavelength of light). In certain embodiments, precursor polymer compositions of the present disclosure comprises one or more photo-initiator elements (i.e., a crosslinking initiator which is initiated or activated by visible light). In certain embodiments, the photo-initiator element can be activated by exposure to light. In certain embodiments, light exposure can activate the photo-initiator to form free-radicals, wherein the free radicals can result in bond formation between reactive groups in the composition, such as vinyl-bond crosslinking between methacrylate groups in a GelMA polymer composition. FIG. 3 provides an example of a series of reactions to produce a GelMA hydrogel polymer composition, in which: (i) a photo-initiator element is activated by light energy (hv) to form free-radicals (R*), which then initiate bond formation between reactive groups on separate gelatin methacryloyl polymer precursors, thereby forming a crosslinked GelMA polymer network. The continued reaction between reactive groups on gelatin methacryloyl components will results in the formation of a broader GelMA hydrogel polymer composition.

In certain embodiments, a photo-initiator element can be activated by exposure to one or more light sources selected from visible light sources (e.g., white or blue light), ultraviolet (UV) light sources, near-infrared (NIR) light sources, and fluorescent light sources. In certain embodiments, the photo-initiator element comprises a visible light-activated photo-initiator, such as a visible light-activated photo-initiator which is activated upon exposure to light having a wavelength from about 380 nm to about 740 nm. In certain embodiments, the visible light-activated photo-initiator can be activated upon exposure to light having a wavelength of from about 380-435 nm (i.e., violet light), about 435-500 nm (i.e. blue light), about 500-565 nm (i.e. green light), about 565-600 nm (i.e. yellow light), about 600-650 nm (i.e. orange light), or about 650-740 nm (i.e. red light). In certain embodiments, the photo-initiator element comprises an ultraviolet light-activated photo-initiator. In certain embodiments, the photo-initiator element comprises a near-infrared (NIR) light-activated photo-initiator. In certain embodiments, the photo-initiator element comprises a white light-activated photo-initiator. In certain embodiments, the photo-initiator element comprises a blue light-activated photo-initiator.

In certain embodiments, methods for producing a gel polymer composition can include a step of adding one or more a therapeutic agent and/or particle (i.e., microparticle or nanoparticle) to the precursor polymer composition. In certain embodiments, one or more a therapeutic agent and/or particle can be added to the precursor polymer before one or more additional chemically-modified polymer precursors with cross-linkable groups are added to the precursor polymer composition. In certain embodiments, one or more a therapeutic agent and/or particle can be added to the precursor polymer before one or more crosslinking agents and/or polymer crosslinking initiators (e.g., photoiniators) are added to the precursor polymer composition. In certain embodiments, methods for producing a gel polymer composition can include: (i) providing a precursor polymer composition comprising chemically-modified gelatin with crosslinkable groups (e.g., acryloyl-substituted gelatin, GelMA); (ii) optionally adding one or more additional chemically-modified polymer precursors with cross-linkable groups to the precursor polymer composition; (iii) adding one or more crosslinking agents and/or polymer crosslinking initiators (e.g., photoiniators) to the precursor polymer; and (iv) optionally adding one or more therapeutic agent and/or particle.

In certain embodiments, a precursor polymer composition can be clarified, purified, or processed for quality and/or purity prior to any polymerizing/crosslinking step. In certain embodiments, a precursor polymer composition can be filtered. In certain embodiments, a precursor polymer composition can be lyophilized. In certain embodiments, a precursor polymer composition can be frozen for storage.

In certain embodiments, methods for producing a gel polymer composition can include a step of polymerizing/crosslinking the precursor polymer composition to produce a gel polymer composition. In certain embodiments, methods for producing a gel polymer composition can include: (i) providing a precursor polymer composition comprising chemically-modified gelatin with crosslinkable groups (e.g., acryloyl-substituted gelatin, GelMA); (ii) optionally adding one or more additional chemically-modified polymer precursors with cross-linkable groups to the precursor polymer composition; (iii) adding one or more crosslinking agents and/or polymer crosslinking initiators (e.g., photoiniators) to the precursor polymer; (iv) optionally adding one or more therapeutic agent and/or particle; and (v) polymerizing/crosslinking the precursor polymer composition to produce a gel polymer composition.

In certain embodiments, the crosslinking of chemically-modified gelatin components and any additional chemically-modified polymer precursors (e.g., MeHA, PEGDA) is initiated, facilitated, or enabled by exposure to UV or visible light in the presence of a photoinitiator component. In certain embodiments, exposure to UV or visible light in the presence of a photoinitiator causes acryloyl groups on one chemically modified gelatin molecule to react with acryloyl groups on other chemically modified gelatin molecules to crosslink the acryloyl-substituted gelatin components and produce a gel (e.g., hydrogel). In certain embodiments, exposure to visible light in the presence of a photoinitiator causes methacryloyl groups on one methacryloyl gelatin molecule to react with methacryloyl groups on other methacryloyl gelatin molecules to crosslink the methacryloyl-substituted gelatin components and produce a gelatin methacryloyl (GelMA) hydrogel.

In certain embodiments, the polymer composition is exposed to a light source for a duration from 1-60 minutes. In certain embodiments, the polymer composition is exposed to a light source for a duration of 1 minute or more, 5 minutes or more, 10 minute or more, 15 minutes or more, 20 minute or more, 25 minutes or more, or 30 minutes or more. In certain embodiments, the polymer composition is exposed to a light source for a duration of 1 minute or less, 5 minutes or less, 10 minute or less, 15 minutes or less, 20 minute or less, 25 minutes or less, or 30 minutes or less, 35 minutes or less, or 40 minutes or less. In certain embodiments, the polymer composition is exposed to a light source for a duration of about 5 seconds, about 10 seconds, about 15 seconds, about 20 seconds, about 25 seconds, about 30 seconds, about 35 seconds, about 40 seconds, about 45 seconds, about 50 seconds, about 55 seconds, about 60 seconds, about 65 seconds, about 70 seconds, about 75 seconds, about 80 seconds, about 85 seconds, about 90 seconds, about 95 seconds, about 100 seconds, about 105 seconds, about 110 seconds, about 115 seconds, about 120 seconds, about 3 minutes, about 4 minutes, about 5 minutes, about 6 minutes, about 7 minutes, about 8 minutes about 9 minutes, about 10 minutes, about 11 minutes about 12 minutes, about 13 minutes, about 14 minutes, about 15 minutes, about 16 minutes, about 17 minutes, about 18 minutes about 19 minutes, about 20 minutes, about 21 minutes about 22 minutes, about 23 minutes, about 24 minutes, about 25 minutes, about 26 minutes, about 27 minutes, about 28 minutes about 29 minutes, about 30 minutes, about 31 minutes about 32 minutes, about 33 minutes, about 34 minutes, about 35 minutes, about 36 minutes, about 37 minutes, about 38 minutes about 39 minutes, about 40 minutes, about 41 minutes about 42 minutes, about 43 minutes, about 44 minutes, about 45 minutes, about 46 minutes, about 47 minutes, about 48 minutes about 49 minutes, about 50 minutes, about 51 minutes about 52 minutes, about 53 minutes, about 54 minutes, about 55 minutes, about 56 minutes, about 57 minutes, about 58 minutes about 59 minutes, or about 60 minutes. In certain embodiments, the polymer composition is exposed to a light source for a duration of from about 1-3 minutes, about 3-6 minutes, about 6-10 minutes, about 1-5 minutes, about 1-10 minutes, about 5-10 minutes, about 11-13 minutes, about 13-16 minutes, about 16-20 minutes, about 10-20 minutes, about 10-15 minutes, about 15-20 minutes, about 21-23 minutes, about 23-26 minutes, about 26-30 minutes, about 20-30 minutes, about 20-25 minutes about 25-30 minutes, about 31-33 minutes, about 33-36 minutes, about 36-40 minutes, about 30-40 minutes, about 30-35 minutes about 35-40 minutes, about 41-43 minutes, about 43-46 minutes, about 46-50 minutes, about 40-50 minutes, about 40-45 minutes, about 45-50 minutes, about 51-53 minutes, about 53-56 minutes, about 56-60 minutes, about 50-60 minutes, about 50-55 minutes, or about 55-60 minutes.

In certain embodiments, a polymer composition can have a thickness from about 1 μm to about 10000 μm. In certain embodiments, a polymer composition can have a thickness from about 1-50 μm, about 50-100 μm, about 100-150 μm, about 150-200 μm, about 200-250 μm, about 250-300 μm, about 300-350 μm, about 350-400 μm, about 400-450 μm, about 450-400 μm, about 400-450 μm, about 450-500 μm, about 500-550 μm, about 550-600 μm, about 600-650 μm, about 650-700 μm, about 700-750 μm, about 750-800 μm, about 800-850 μm, about 850-900 μm, about 900-950 μm, about 950-1000 μm, about 1000-1500 μm, about 1500-2000 μm, about 2000-2500 μm, about 2500-3000 μm, about 3000-3500 μm, about 3500-4000 μm, about 4000-4500 μm, about 4500-4000 μm, about 4000-4500 μm, about 4500-5000 μm, about 5000-5500 μm, about 5500-6000 μm, about 6000-6500 μm, about 6500-7000 μm, about 7000-7500 μm, about 7500-8000 μm, about 8000-8500 μm, about 8500-9000 μm, about 9000-9500 μm, or about 9500-10000 μm.

In certain embodiments, a precursor polymer compositions can be cooled prior to or during crosslinking reactions. In certain embodiments, a precursor polymer compositions can be cooled to a temperature of from about 0° C. and about 30° C. prior to or during crosslinking reactions. In certain embodiments, a precursor polymer compositions can be cooled to a temperature of from about 0-5° C., about 5-10° C., about 0-10° C., about 10-15° C., about 15-20° C., about 10-20° C., about 20-25° C., about 25-30° C., or about 20-30° C. In certain embodiments, a precursor polymer compositions can be heated prior to or during crosslinking reactions. In certain embodiments, a precursor polymer compositions can be heated to a temperature of from about 30° C. and about 150° C. prior to or during crosslinking reactions. In certain embodiments, a precursor polymer compositions can be heated to a temperature of from about 30-35° C. about 35-40° C., about 30-40° C., about 40-45° C., about 45-50° C., about 40-50° C., about 50-55° C., about 55-60° C., about 50-60° C., about 60-65° C., about 65-70° C., about 60-70° C., about 70-75° C., about 75-80° C., about 70-80° C., about 80-85° C. about 85-90° C., about 80-90° C. about 90-95° C., about 95-100° C., about 90-100° C. about 100-105° C., about 105-110° C., about 100-110° C., about 110-115° C., about 115-120° C., about 110-120° C., about 130-135° C., about 135-140° C., about 130-140° C., about 140-145° C., about 145-150° C., or about 140-150° C.

Once crosslinking reactions are completed or halted, the resulting gel polymer materials can be clarified, purified, or processed for quality, purity, and/or therapeutic viability. In certain embodiments, a gel polymer composition can be dialyzed to remove any unreacted compounds from the gel mixture or structure. In certain embodiments, a gel polymer composition can be dialyzed with a dialysis buffer that comprises deionized water. In certain embodiments, a gel polymer composition can be filtered. In certain embodiments, a gel polymer composition can be dried. In certain embodiments, a gel polymer composition can be lyophilized. In certain embodiments, a gel polymer composition can be frozen for storage.

In certain embodiments, polymer compositions of the present disclosure can be formed, molded, extruded woven, or otherwise produced or processed into fibers, films, discs, fabrics, tubes, conduits, rods, rings, mesh, or any other form or shape for polymeric or gel materials known in the art. In certain embodiments, polymer compositions of the present disclosure can be formed, molded, extruded woven, or otherwise produced or processed into single layer structures or multi-layered structures (e.g., two layers, three layers, four layers, etc.).

In certain embodiments, a polymer composition of the present disclosure comprises macromolecular polymeric and/or fibrous elements which are interwoven or intertwined within the interstitial porous network of a polymer composition, but which are not chemically connected to the core crosslinked polymeric network. Non-limiting examples of such macromolecules include polycaprolactone, gelatin, gelatin methacrylate, alginate, alginate methacrylate, chitosan, chitosan methacrylate, glycol chitosan, glycol chitosan methacrylate, hyaluronic acid, hyaluronic acid methacrylate, and other non-crosslinked natural or synthetic polymeric chains. A gel materials which includes an interwoven macromolecular structure can be referred to as a composite structure or composite gel. Examples of hydrogel/fiber composites are described, for example, in Moutos et al. Nat. Mater., 2007, 6(2), p. 162-7; which is incorporated herein by reference in its entirety, insofar as it describes the composition, production, analysis and use of composite gel materials. In certain embodiments, a precursor polymer composition can be in a high-viscosity form (e.g., paste-like viscosity), and incorporated into a macromolecular polymeric matrix (e.g., fibrous mat or tissue matrix). In certain embodiments, a precursor polymer composition can be in a low-viscosity form (e.g., liquid-like viscosity), and incorporated into a macromolecular polymeric matrix (e.g., fibrous mat or tissue matrix).

In certain embodiments, a cross-linked polymer composition can have a substantially covalent matrix form. In certain embodiments, a cross-linked polymer composition can have an amorphous matrix form (i.e., matrix formed primarily through ionic and/or hydrogen bonding).

In certain embodiments, polymer compositions of the present disclosure can be formed as patterned gel compositions (e.g., a micropatterned hydrogel). Micropatterned hydrogels can be prepared using, for example, the methods set forth in U.S. Pat. No. 6,423,252, which is incorporated herein by reference in its entirety, insofar as it describes the composition, production (including micropatterning), analysis and use of hydrogels, including acrylated gelatin polymeric compositions such as GelMA hydrogels. For example, the method comprises: (i) contacting a precursor polymer composition with a mold or surface which comprises a three-dimensional negative configuration (i.e., template) of a micropattern; and (ii) crosslinking and/or polymerizing the precursor polymer composition to produce a crosslinked gel polymer composition (e.g., GelMA hydrogel) which includes the micropattern on at least on surface of the hydrogel.

In certain embodiments, polymer compositions of the present disclosure can be formed as molded, stamped, or shaped gel compositions. Molded, stamped or shaped hydrogels can be prepared using, for example, the methods set forth in US 20050008675 or US 20040258729, each of which is incorporated herein by reference in its entirety, insofar as each describes the composition, production (including molding), analysis and use of hydrogels, including acrylated gelatin polymeric compositions such as GelMA hydrogels.

### IV. Administration and Treatments

### General

Suturing, tissue transplantation, and the use of tissue adhesives are common treatments for defects and/or traumatic injuries to soft tissues (such as corneal or scleral tissues). However, each treatment carries risks and complications: (i) Suturing requires advanced surgical skill and early treatment, it often results in irregular stigmatisms, and can often lead to microbial entrapment and infection; (ii) Tissue grafting and transplantation require donor tissue (with associated high costs), advanced surgical skill, and present a high risk of immune reactions or full rejection of the grafted tissue; (iii) Tissue adhesives (such as cyanoacrylate glues, fibrin glues, or polyethylene-glycol (PEG)-based sealants) have limited effectiveness and adhesion (particularly in aqueous and physiological environments), have limited durability, can be difficult to apply and control texture, have a high probability of leaking, lack of biocompatibility (e.g., inflammatory) and possible toxicity, have a lack of translucence/transparency, have a high risk of infection (including risks related to high porosity), and have generally not received FDA safety approval for alleviating corneal defects or repairing corneal incisions, perforations or trauma.

There remains a need for improved polymer compositions which are effective in treating and/or sealing injuries, defects, and diseases to soft tissues in subjects (i.e., tissue of the body except bone).

In certain embodiments, polymer compositions of the present disclosure can be used as a sealant composition for treating or repairing soft tissue in a subject. In certain embodiments, polymer compositions of the present disclosure can be used as a delivery vehicle for administering a therapeutic agent for treating or repairing soft tissue in a subject. In certain embodiments, polymer compositions of the present disclosure can be used as a sealant composition for treating or repairing soft tissue in a subject, and as a delivery vehicle for administering a therapeutic agent for treating or repairing the soft tissue of the subject.

In certain embodiments, the methods and compositions of the present disclosure can be used to adhere, seal or treat target soft tissues of a subject. In certain embodiments, the methods and compositions of the present disclosure can be used to adhere, seal or treat one or more target soft tissues selected from: adipose tissue, bladder tissue, bone marrow, cardiovascular tissue (e.g., cardiac), dura mater, endocrine glands, gastrointestinal tissue, hair follicles, kidney tissue, liver tissue, lung tissue, lymph nodes, muscle tissue, neural/nerve tissue (e.g., peripheral nervous system), ocular tissue (e.g., corneal), oral tissue (e.g., craniofacial, odontic, periodontic), pancreatic tissue, renal tissue, skin tissue (e.g., for treatment of topical ulcers, such as diabetic ulcers), urethra tissue, vascular tissue. In certain embodiments, the methods and compositions of the present disclosure can be used to adhere, seal, or treat one or more target soft tissues in stressed and/or physiological environment, or similar applications which require elastic and/or adhesive compositions.

Polymer compositions (e.g., GelMA polymer compositions) of the present disclosure may be administered by any route which results in a therapeutically effective outcome.

In certain embodiments, the method includes applying a pre-gelation polymer composition to an applicator; placing the applicator containing the pre-gelation polymer composition onto a surface of the target tissue of the subject; and crosslinking (e.g., photo-crosslinking) the polymer composition by exposing the pre-gelation polymer composition to crosslinking conditions (e.g., visible light with a photoinitiator). In certain embodiments, the pre-gelation polymer composition is applied directly to the surface of the target tissue without an applicator. In certain embodiments, application to the surface of a target tissue comprises application to an external surface of a target tissue (e.g., topical application). In certain embodiments, application to the surface of a target tissue comprises application/injection to a space directly below the surface of a target tissue (e.g., subconjunctival application to ocular tissue, subretinal application to ocular tissue).

In certain embodiments, a target soft tissue can be treated or sealed by applying a first layer which comprises a first polymer composition of the present disclosure which is engineered to have specific physical, mechanical, structural, chemical and/or biological properties (e.g., elasticity, biodegradability, porosity); and then applying a second layer which comprises a second polymer composition which is engineered to have different physical, mechanical, structural, chemical and/or biological properties (e.g., elasticity, biodegradability, porosity). In certain embodiments, the method can include applying one or more additional layers (e.g., a third layer, a fourth layer, etc), each of which comprises a polymer composition of the present disclosure which is engineered to have specific physical, mechanical, structural, chemical and/or biological properties (e.g., elasticity, biodegradability, porosity).

In certain embodiments, a target soft tissue can be treated by: (i) forming a pre-formed polymer composition by polymerizing a polymer composition of the present disclosure; and (ii) applying the pre-formed polymer composition onto a surface or under the surface e.g., subconjunctival, subretinal) of the target tissue of the subject. In certain embodiments, application to the surface of a target tissue (e.g., onto or under the surface) comprises application/injection to a space directly below the surface of a target tissue (e.g., subconjunctival application to ocular tissue, subretinal application to ocular tissue). In certain embodiments, the pre-formed polymer composition can be engineered to have specific physical, mechanical, structural, chemical and/or biological properties (e.g., elasticity, biodegradability, porosity).

In certain embodiments, a target soft tissue can be treated by: (i) forming a pre-formed hydrogel polymer composition by polymerizing a polymer composition of the present disclosure; (ii) drying the hydrogel polymer by removing a substantial portion of interstitial fluid from the hydrogel (e.g., at least 50%, at least 60%, at least 70%, at least 80%, at least 90%, or at least 95% of interstitial fluid); (iii) applying the pre-formed polymer composition onto a surface or under the surface e.g., subconjunctival, subretinal) of the target tissue of the subject; and (iv) optionally rehydrating the dried hydrogel polymer to a substantially hydrated form (e.g., e.g., at least 50%, at least 60%, at least 70%, at least 80%, at least 90%, or at least 95% of interstitial fluid volume). In certain embodiments, application to the surface of a target tissue (e.g., onto or under the surface) comprises application/injection to a space directly below the surface of a target tissue (e.g., subconjunctival application to ocular tissue, subretinal application to ocular tissue). In certain embodiments, the pre-formed polymer composition can be engineered to have specific physical, mechanical, structural, chemical and/or biological properties (e.g., elasticity, biodegradability, porosity).

### Therapeutic Compositions

In certain embodiments, polymer compositions of the present disclosure can be prepared as, or comprised in, therapeutic compositions. In certain embodiments, hydrogel polymer compositions of the present disclosure can be prepared as, or comprised in, therapeutic compositions. In certain embodiments, GelMA hydrogel polymer compositions of the present disclosure can be prepared as, or comprised in, therapeutic compositions. Such compositions comprises one or more polymer composition of the present disclosure (including, optionally, one or more therapeutic agents or active ingredients) and one or more therapeutically acceptable excipients (e.g., carrier, solvent, or delivery vehicle).

Relative amounts of the polymer compositions (e.g., GelMA hydrogel polymer composition), a therapeutically acceptable excipient, and/or any additional ingredients in a therapeutic composition in accordance with the present disclosure may vary, depending upon the identity, size, and/or condition of the subject or tissue being treated and further depending upon the route by which the composition is to be administered or applied. In certain embodiments, a therapeutic composition comprises from 0.10% and 99% (w/v) of a polymer composition of the present disclosure in the volume of the therapeutic composition. In certain embodiments, a therapeutic composition comprises a polymer composition of the present disclosure at weight-per-volume concentration (w/v) of about 0.5%, about 1%, about 2%, about 3%, about 4%, about 5%, about 6%, about 7%, about 8%, about 9%, about 10%, about 11% about 12%, about 13%, about 14%, about 15%, about 16%, about 17%, about 18%, about 19%, about 20%, about 21% about 22%, about 23%, about 24%, about 25%, about 26%, about 27%, about 28%, about 29%, about 30%, about 31% about 32%, about 33%, about 34%, about 35%, about 36%, about 37%, about 38%, about 39%, about 40%, about 41% about 42%, about 43%, about 44%, about 45%, about 46%, about 47%, about 48%, about 49%, about 50%, about 51% about 52%, about 53%, about 54%, about 55%, about 56%, about 57%, about 58%, about 59%, about 60%, about 61% about 62%, about 63%, about 64%, about 65%, about 66%, about 67%, about 68%, about 69%, about 70%, about 71% about 72%, about 73%, about 74%, about 75%, about 76%, about 77%, about 88%, about 79%, about 80%, about 81% about 82%, about 83%, about 84%, about 85%, about 86%, about 87%, about 88%, about 89%, about 90%, about 91% about 92%, about 93%, about 94%, about 95%, about 96%, about 97%, about 98%, or about 99%. In certain embodiments, a therapeutic composition comprises a polymer composition of the present disclosure at weight-per-volume concentration (w/v) of from about 1-3%, about 3-6%, about 6-10%, about 1-5%, about 5-10%, about 1-10%, about 11-13%, about 13-16%, about 16-20%, about 10-15%, about 15-20%, about 10-20%, about 21-23%, about 23-26%, about 26-30%, about 20-25%, about 25-30%, about 20-30%, about 31-33%, about 33-36%, about 36-40%, about 30-35% about 35-40%, about 30-40%, about 41-43%, about 43-46%, about 46-50%, about 40-45%, about 45-50%, about 40-50%, about 51-53%, about 53-56%, about 56-60%, about 50-55%, about 55-60%, about 50-60%, about 61-63%, about 63-66%, about 66-70%, about 60-65%, about 65-70%, about 60-70%, about 71-73%, about 73-76%, about 76-80%, about 70-75%, about 75-80%, about 70-80%, about 81-83%, about 83-86%, about 86-90%, about 80-85% about 85-90%, about 80-90%, about 91-93%, about 93-96%, about 96-99%, about 90-95%, about 95-99%, or about 90-99%.

In certain embodiments, therapeutic compositions and formulations of the present disclosure comprises, without limitation, saline, liposomes (e.g., unilamellar vesicles, multilamellar vesicles), lipid particles (including microparticles and nanoparticles), and/or polymeric particles (including microparticles and nanoparticles). In certain embodiments, therapeutic compositions and formulations of the present disclosure comprises a polymeric composition of the present disclosure which incorporates, without limitation, saline, liposomes, lipid particles (including microparticles and nanoparticles), polymeric particles (including microparticles and nanoparticles) or a combination thereof.

In certain embodiments, therapeutic compositions and formulations of the present disclosure are aqueous formulations (i.e., formulations which comprise water). In certain embodiments, therapeutic compositions and formulations of the present disclosure comprise water, sanitized water, or Water-for-injection (WFI).

In certain embodiments, therapeutic compositions and formulations of the present disclosure comprises one or more of the following: pH buffered solutions (e.g., phosphate buffered saline (PBS), HEPES, TES, MOPS), isotonic saline, Ringer's solution, polyols (e.g., glycerol, propylene glycol, liquid polyethylene glycol), alginic acid, ethyl alcohol, and therapeutically acceptable mixtures thereof. In certain embodiments, therapeutic compositions and formulations of the present disclosure comprises phosphate buffered saline (PBS).

Formulations of the present disclosure can be used in any step of producing, processing, preparing, storing, expanding, or administering polymer compositions of the present disclosure.

In certain embodiments, therapeutic compositions of the present disclosure comprises one or more therapeutically acceptable excipient (e.g., a vehicle capable of suspending or dissolving the polymeric compound. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: acetic acid, aluminium stearate, butylated hydroxytoluene (BHT), calcium carbonate, calcium chloride, calcium phosphate (dibasic), calcium stearate, carboxymethyl celluloses, croscarmellose, crosslinked polyvinyl pyrrolidone, citric acid, crospovidone, cysteine, ethylcellulose, gelatin, glucose, glucuronic acid, gluconic acid, hydroxypropyl cellulose, hydroxypropyl methylcellulose, hydroxyl-butanedioic acid, inosite, lactose, magnesium chloride, magnesium stearate, maltitol, mannitol, methionine, methylcellulose, methyl paraben, microcrystalline cellulose, phosphoric acid, polyethylene glycol, polyvinyl pyrrolidone, povidone, pregelatinized starch, propyl paraben, retinyl palmitate, saccharose, shellac, silicon dioxide, sodium acetate, sodium carbonate, sodium bicarbonate, sodium carboxymethyl cellulose, sodium chloride, sodium citrate, sodium hydroxide, sodium phosphate, sodium starch glycolate, sorbitol, starch (corn), stearic acid, sucrose, talc, titanium dioxide, vitamin A, vitamin E, vitamin C, xylitol, zinc stearate, and combinations thereof.

### Therapeutic Agents

In certain embodiments, the polymer compositions of the present disclosure can include a therapeutic agent. In certain embodiments, the polymer compositions of the present disclosure can include a therapeutic agent as a delivery payload.

In certain embodiments, a polymer compositions of the present disclosure can include a therapeutic agent at a concentration (w/v) from about 0% and about 40%. In certain embodiments, a precursor polymer compositions of the present disclosure can include a therapeutic agent at a concentration (w/v) from about 0% and about 40%. In certain embodiments, a gel polymer compositions of the present disclosure can include a therapeutic agent at a concentration (w/v) from about 0% and about 40%. In certain embodiments, a polymer compositions of the present disclosure can include a therapeutic agent at a concentration (w/v) from about 1-2%, about 2-4%, about 4-6%, about 6-8%, about 8-10%, about 1-5%, about 5-10%, about 1-10%, 10-12%, about 12-14%, about 14-16%, about 16-18%, about 18-20%, about 10-15%, about 15-20%, about 10-20%, about 20-22%, about 22-24%, about 24-26%, about 26-28%, about 28-30%, about 20-25%, about 25-30%, about 20-30%, about 30-32%, about 32-34%, about 34-36%, about 36-38%, about 38-40%, about 30-35%, about 35-40%, or about 30-40%.

In certain embodiments, a precursor polymer compositions of the present disclosure can include a therapeutic agent at a concentration from about 0.1 mg/mL and about 500 mg/mL. In certain embodiments, a polymer compositions of the present disclosure can include a therapeutic agent at a concentration from about 0.1-0.5 mg/mL, about 0.5-1.0 mg/mL, about 1.0-2.5 mg/mL, about 2.5-5.0 mg/mL, about 5.0-10.0 mg/mL, about 10.0-25.0 mg/mL, about 25.0-50.0 mg/mL, about 50.0-100.0 mg/mL, about 100-150 mg/mL, about 150-200 mg/mL, about 200-250 mg/mL, about 250-300 mg/mL, about 300-350 mg/mL, about 350-400 mg/mL, about 400-450 mg/mL, about 450-500 mg/mL, about 500-550 mg/mL, about 550-600 mg/mL, about 600-650 mg/mL, about 650-700 mg/mL, about 700-750 mg/mL, about 750-800 mg/mL, about 800-850 mg/mL, about 850-900 mg/mL, about 900-950 mg/mL, or about 950-1000 mg/mL.

In certain embodiments, a polymer composition can deliver a therapeutic agent to a peak concentration in less than 1 hour. In certain embodiments, a polymer composition can deliver a therapeutic agent to a peak concentration in less than 1 day. In certain embodiments, a polymer composition can deliver a therapeutic agent to a peak concentration in from about 0-2 hours, about 2-4 hours, about 4-6 hours, about 6-8 hours, about 8-10 hours, about 10-12 hours, about 12-16 hours, about 16-20 hours, about 20-24 hours, about 24-30 hours, about 30-36 hours, about 36-42 hours, or about 42-48 hours. In certain embodiments, a polymer composition can deliver a therapeutic agent to a peak concentration in less than 1 week. In certain embodiments, a polymer composition can deliver a therapeutic agent to a peak concentration in from about 0-2 days, about 2-4 days, about 4-6 days, about 6-8 days, about 8-10 days, about 10-12 days, about 12-16 days, about 16-20 days, about 20-24 days, about 24-30 days, about 30-35 days, about 35-40 days, about 40-45 days, about 45-50 days, about 50-55 days, about 55-60 days. In certain embodiments, a polymer composition can deliver a therapeutic agent to a peak concentration in less than 1 month. In certain embodiments, a polymer composition can deliver a therapeutic agent to a peak concentration in less than 12 months. In certain embodiments, a polymer composition can deliver a therapeutic agent to a peak concentration in from about 0-1 months, about 1-2 months, about 2-3 months, about 3-4 months, about 4-5 months, about 5-6 months, about 6-7 months, about 7-8 months, about 8-9 months, about 9-10 months, about 10-11 months, or about 11-12 months.

In certain embodiments, the therapeutic agent comprises one or more of a growth factor, a hemostatic agent, analgesics, anesthetics, antifungals, antibiotics, antibacterials, antiinflammatories, antimicrobials, anthelmintics, antidotes, antiemetics, antihistamines, antihypertensives, antimalarials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, immunosuppressants, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, organic molecules, small molecule inhibitors, glycosaminoglycans, organometallic agents, chelated metals or metal salts, peptide-based drugs, vitamins, nutritional supplements, glycoproteins (e.g., collagen), extracellular matrix proteins or fragments thereof, fibronectin, peptides and/or proteins, polysaccharides, carbohydrates (both simple and/or complex), proteoglycans, antigens, oligonucleotides (sense and/or antisense DNA and/or RNA), antibodies, nucleic acid sequences, gene therapy agents, triamcinolone acetonide, ovalbumin, or a combination thereof.

In certain embodiments, the therapeutic agent comprises one or more anti-acanthamoebal, antiviral and/or antibacterial agents. In certain embodiments, the therapeutic agent comprises one or more agent selected from acyclovir, valacyclovir, famciclovir, penciclovir, trifluridine, vidarabine, hydroxychloroquine, gatifloxacin, daptomicin, tigecycline, telavancin, chloramphenicol, fusidic acid, chlorohexidine, polyhexamethylen biguanide, propamidine, hexamidine, bacitracin, metronidazole, rifampin, ethambutol, streptomycin, isoniazid, silver nanoparticles, copper oxide nanoparticles, glicopeptides (e.g., teicoplanin, vancomycin), aminoglicosydes (e.g., gentamycin, tobramycin, amikacin, netimicin), cephalosporins (e.g., cefazolin, cefoxitin, cefotaxime, cefuroxime, moxalactam), macrolids (e.g., erythromycin), oxazolidinones (e.g., linezolid), quinolones, polymixins, sulfonamides, tetracyclines, penems, carbapenems, monobactams, lincosides, spectinomycin, clindamycin, ansamycins, daptomycin, nitrofurans, trimethoprim sulfamethoxazole, chitosan, penicillin, ciprofloxacin, or a combination thereof.

In certain embodiments, the therapeutic agent comprises one or more anti-fungal agents. In certain embodiments, the therapeutic agent comprises one or more agent selected from amphotericin B, natamycin, candicin, filipin, hamycin, nystatin, rimocidin, voriconazole, imidazoles, triazoles, thiazoles, allylamines, echinocandins, benzoic acid, ciclopirox, flucytosine, griseofulvin, haloprogin, tolnaftate, undecylenic acid, and povidone iodine, or a combination thereof.

In certain embodiments, the therapeutic agent comprises one or more antimicrobial agents. In certain embodiments, the therapeutic agent comprises one or more antimicrobial agents selected from polymyxin B, vancomycin, cholera toxin, diphtheria toxin, lysostaphin, hemolysin, bacitracin, boceprevir, albavancin, daptomycin, enfuvirtide, oritavancin, teicoplanin, telaprevir, telavancin, guavanin 2, Maximin H5, dermcidin, cecropins, andropin, moricin, ceratotoxin, melittin, magainin, dermaseptin, brevinin-1, esculentins, buforin II CAP18, LL37, baecin, apidaecins, prophenin, indolicidin, antimicrobial peptide (AMP) (e.g., Tet213), chlorhexidine, a chlorhexadine salt, triclosan, polymyxin, tetracycline, an amino glycoside (e.g., gentamicin, Tobramycin), rifampicin, erythromycin, neomycin, chloramphenicol, miconazole, a quinolone, penicillin, fusidic acid, cephalosporin, mupirocin, metronidazole, secropin, protegrin, bacteriolcin, defensin, nitrofurazone, mafenide, aracyclovir, clindamycin, lincomycin, sulfonamide, norfloxacin, pefloxacin, nalidizic acid, cinnamycin, anti-DEFA5, duramycin, nisin, pediocin, Abaecin, Ct-AMP1, Apidaecin IA, Apidaecin IB, Bactenecin, Bactenecin 5, Bactenecin 7, Bactericidin B-2, Aurein family, SMAP-29, Temporin B, Pleurocidin, Tachyplesin III, LL-37, Citropin 1.1, BMAP-27, BMAP-28, Agelaia-MP, Temporin 101a, NA-CATH, Histatins, Latarcin, Halocidin, Bombinin, Cathelicidin, Malacidin, MP196, MS100a7a15, Murepavadin, Myticin, Mytilin, Paenibacterin, Pardaxin, Peptaibol, SAAP-148, Sarcotoxin, Stomoxyn, Tachyplesin, thioester-containing protein 1, Thionin, Alamethicin, Arenicin, dermorphins, deltorphins, dermaseptins, pseudin, bombesins, maculatins, LEAP2, Efrapeptin, Arylomycins, Capreomycin, Gramicidin B, Antiamoebin, Bacillomycin, Teixobactin, Tyrothricin, Viomycin, oxalic acid, or combinations thereof.

In certain embodiments, the therapeutic agent comprises one or more anti-inflammatory agents. In certain embodiments, the therapeutic agent comprises one or more anti-inflammatory agent selected from steroidal anti-inflammatory drugs (e.g., prednisolone), corticosteroids (e.g., loteprednol etabonate), salicylates, non-steroidal anti-inflammatory drugs (e.g., bromfenac), mTOR inhibitors, calcineurin inhibitors, synthetic or natural anti-inflammatory proteins, dexamethasone, 5-fluorouracil, daunomycin, paclitaxel, curcumin, resveratrol, mitomycin, methylprednisolone, prednisolone, hydrocortisone, fludrocortisone, prednisone, celecoxib, ketorolac, piroxicam, diclorofenac, ibuprofen, and ketoprofen, rapamycin, cyclosporin, tacrolimus/FK-506, or a combination thereof.

In certain embodiments, the therapeutic agent comprises one or more growth factors. In certain embodiments, the therapeutic agent comprises a growth factor which comprises a recombinant hepatocyte growth factor or recombinant nerve growth factor. In certain embodiments, the therapeutic agent comprises one or more growth factors selected from Activins (e.g., Activin A, Activin B, Activin AB), Adrenomedullin (AM), albumin, alpha-2 macroglobulin, annexin, Angiopoietin (Ang), Artemin, Autocrine motility factor, Bone morphogenetic proteins (BMPs) (e.g., BMP-1, BMP-2, BMP-3, BMP-4, BMP-5, BMP-6, BMP-7, BMP-8, BMP-9), Brain-derived neurotrophic factor (BDNF), Ciliary neurotrophic factor family, Ciliary neurotrophic factor (CNTF), connective tissue activated peptides (CTAPs), Epidermal growth factor (EGF), Ephrins (e.g., Ephrin A1, Ephrin A2, Ephrin A3, Ephrin A4, Ephrin A5, Ephrin B1, Ephrin B2, Ephrin B3), Erythropoietin (EPO), Fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), basic fibroblast growth factor (bFGF), acidic fibroblast growth factor (aFGF), Fetal Bovine Somatotrophin (FBS), Glial cell line-derived neurotrophic factor (GDNF), Granulocyte colony-stimulating factor (G-CSF), Granulocyte macrophage colony-stimulating factor (GM-CSF), Growth differentiation factors (GDF) (e.g., GDF1, GDF9), Heparin-binding growth factors, Hepatocyte growth factor (HGF), Hepatocyte growth factor-like protein (HGFLP), Hepatoma-derived growth factor (HDGF), Inhibins (e.g., Inhibin A, Inhibin B), Insulin, Insulin-like growth factor (IGF) (e.g., IGF-1, IGF-2), Interleukins (IL) (e.g., IL-1, IL-2, IL-3, IL-4, IL-5, IL-6, IL-7, IL-8, IL-11, and IL-13), Keratinocyte growth factor (KGF), Leukemia inhibitory factor (LIF), Macrophage colony-stimulating factor (M-CSF), Macrophage-stimulating protein (MSP), Migration-stimulating factor (MSF), Myostatin, Neuregulins (NRG) (e.g., NRG1, NRG2, NRG3, NRG4), Neurotrophins (NT) (e.g., NT-1, NT-2, NT-3, NT-4), Neurturin, Nerve growth factor (NGF), osteogenic factors, Persephin, Placental growth factor (PGF), Platelet-derived growth factor (PDGF), Renalase (RNLS), stromal cell-derived factor-1, T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF), anti-vascular endothelial growth factor (anti-VEGF) (e.g., bevacizumab, ranibizumab, aflibercept), and biologically active analogs, fragments, derivatives of such growth factors, or a combination thereof.

In certain embodiments, the therapeutic agent comprises one or hormone. In certain embodiments, the therapeutic agent comprises one or more hormones selected from: antimullerian hormone, mullerian inhibiting factor or hormone), adiponectin, adrenocorticotropic hormone, corticotropin, angiotensinogen, angiotensin, antidiuretic hormone, vasopressin, arginine vasopressin, atrial-natriuretic peptide, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, somatomedin, leptin, luteinizing hormone, melanocyte stimulating hormone, orexin, oxytocin, parathyroid hormone, prolactin, relaxin, secretin, somatostatin, thrombopoietin, thyroid-stimulating hormone, thyrotropin, thyrotropin-releasing hormone, or combinations thereof.

In certain embodiments, a polymer compositions of the present disclosure can include one or more growth factors at a concentration (w/v) from about 0.001 μg/mL and about 2 μg/mL. In certain embodiments, a polymer compositions of the present disclosure can include one or more growth factors at a concentration (w/v) from about 0.001 μg/mL and about 1000 μg/mL. In certain embodiments, a polymer compositions can include one or more growth factors at a concentration (w/v) from about 0.01 μg/mL and about 500 μg/mL. In certain embodiments, a polymer compositions can include one or more growth factors at a concentration (w/v) from about 0.1 μg/mL and about 200 μg/mL. In certain embodiments, a polymer compositions can include one or more growth factors at a concentration (w/v) from about 0.1-0.5 μg/mL, about 0.5-1.0 μg/mL, about 1-2 μg/mL, about 2-4 μg/mL, about 4-6 μg/mL, about 6-8 μg/mL, about 8-10 μg/mL, about 10-12 μg/mL, about 12-14 μg/mL, about 14-16 μg/mL, about 16-18 μg/mL, about 18-20 μg/mL, about 20-22 μg/mL, about 22-24 μg/mL, about 24-26 μg/mL, about 26-28 μg/mL, about 28-30 μg/mL, about 30-35 μg/mL, about 35-40 μg/mL, about 40-45 μg/mL, about 45-50 μg/mL, about 50-55 μg/mL, about 55-60 μg/mL, about 60-65 μg/mL, about 65-70 μg/mL, about 70-75 μg/mL, about 75-80 μg/mL, about 80-85 μg/mL, about 85-90 μg/mL, about 90-95 μg/mL, about 95-100 μg/mL, about 100-125 μg/mL, about 125-150 μg/mL, about 150-175 μg/mL, about 175-200 μg/mL, about 200-225 μg/mL, about 225-250 μg/mL, about 250-275 μg/mL, about 275-300 μg/mL, about 300-325 μg/mL, about 325-350 μg/mL, about 350-375 μg/mL, about 375-400 μg/mL, about 400-425 μg/mL, about 425-450 μg/mL, about 450-475 μg/mL, about 475-500 μg/mL, about 500-550 μg/mL, about 550-600 μg/mL, about 600-650 μg/mL, about 650-700 μg/mL, about 700-750 μg/mL, about 750-800 μg/mL, about 800-850 μg/mL, about 850-900 μg/mL, about 900-950 μg/mL, about 950-1000 μg/mL, about 1000-1100 μg/mL, about 1100-1200 μg/mL, about 1200-1300 μg/mL, about 1300-1400 μg/mL, about 1400-1500 μg/mL, about 1500-1600 μg/mL, about 1600-1700 μg/mL, about 1700-1800 μg/mL, about 1800-1900 μg/mL, or about 1900-2000 μg/mL,

In certain embodiments, the therapeutic agent comprises one or more hemostatic agents (i.e. a material that promotes hemostasis) and/or immunosuppressive agents. In certain embodiments, the therapeutic agent comprises one or more agents selected from blood platelets, platelet-like nanoparticles (e.g., silicate nanoparticles), blood coagulation factors (e.g., thrombin, prothrombin), alkylating agents, antimetabolites, mycophenolate, cyclosporine, tacrolimus, rapamycin, or combinations thereof. In certain embodiments, the therapeutic agent comprises an anticoagulant or blood thinner (e.g., heparin).

In certain embodiments, a polymer compositions of the present disclosure can incorporate or be coated with cells or cell-precursors of a target tissue. In certain embodiments, a polymer compositions can incorporate or be coated with one or more cells or cell-precursors of a target tissue selected from nerve cells, muscle cells, myocytes, cardiomyocytes, hepatocytes, keratinocytes, melanocytes, ameloblasts, fibroblasts, preosteoblasts, osteoblasts, osteoclasts, endothelial cells, epithelial cells, mesenchymal stem cells, neurolemmocytes (i.e., Schwann cells), embryonic stem cells, adult stem cells, pluripotent stem cells, multipotent stem cells, hematopoietic stem cells, adipose derived stem cells, bone marrow derived stem cells, osteocytes, neurocytes, or a combination thereof. In certain embodiments, a polymer composition can incorporate or be coated with endothelial cells (e.g., corneal endothelial cells). In certain embodiments, a polymer composition can incorporate or be coated with ocular cells. In certain embodiments, a polymer composition can incorporate or be coated with adherent cell types (i.e., cells that form cell-to-cell network, 3D vasculature). In certain embodiments, a polymer composition can incorporate or be coated with monolayer cell types (i.e., 2D). In certain embodiments, a polymer composition can incorporate or be coated with epithelial cells, endothelial cells, keratocytes, and combinations thereof. In certain embodiments, a polymer composition can incorporate or be coated with human umbilical vein endothelial cells (HUVEC) or vascular endothelial cells. In certain embodiments, a polymer composition can incorporate or be coated with human retinal pigment epithelium cells (HRPEC), human neuroepithelial cells, human photoreceptor cells, human corneal endothelial cells, human neural crest cells, human retinal ganglion cells, human limbal cells, human cardiomyocytes, human hepatocytes, human dermal cells, human gastrointestinal epithelial cells, human neurons, and human islet cells, human immune cells, and other therapeutic human cells.

In certain embodiments, cells or cell-precursors can be incorporated into or onto a polymer gel matrix by placing the polymer gel composition in a cell culture mixture for a duration of time. The culture time may differ depending upon the cells used, but can generally be 1 to 21 days. In certain embodiments, exposure of the polymer gel composition to cell cultures is repeated to increase the cell density in or on the gel matrix.

In certain embodiments, a polymer compositions of the present disclosure can incorporate cells or cell-precursors according to the procedures disclosed in WO 2013040559; or Loessner et al., Nature protocols. 2016 April; 11(4):727. A1; each of which is incorporated herein by reference in its entirety, insofar as each describes the incorporation of cells or cell-precursors onto or into a gel matrix, such as a GelMA hydrogel.

### Therapeutic Applications

In certain embodiments, polymer compositions of the present disclosure can be used as a sealant and/or therapeutic composition for treating and/or repairing soft tissue in a subject. In certain embodiments, polymer compositions of the present disclosure can be used as a delivery vehicle for administering a therapeutic agent for treating and/or repairing soft tissue in a subject. In certain embodiments, polymer compositions of the present disclosure can be used as a sealant and/or therapeutic composition for treating and/or repairing soft tissue in a subject, and as a delivery vehicle for administering a therapeutic agent for treating and/or repairing the soft tissue of the subject.

In certain embodiments, the methods and compositions of the present disclosure can be used to adhere, seal or treat one or more target soft tissues selected from ocular tissue (i.e. eyes), lung, cardiovascular, skin, kidney, bladder, urethra, dura mater, liver, gastrointestinal, or oral (i.e. mouth) tissue. In certain embodiments, the methods and compositions of the present disclosure can be used to adhere, seal or treat one or more target soft tissues in a stressed and/or physiological environment, or similar applications which require elastic and/or adhesive compositions.

In certain embodiments, the present disclosure provides methods for treating and/or repairing soft tissue in a subject using a polymer compositions of the present disclosure. In certain embodiments, the present disclosure provides methods for treating and/or repairing a defect, injury, and/or disease in the soft tissue of a subject using a polymer compositions of the present disclosure. In certain embodiments, the method includes: applying a pre-gelation polymer composition of the present disclosure (e.g., a polymer composition comprising acryloyl-substituted gelatin) to an applicator; placing the applicator containing the pre-gelation polymer composition onto a surface of a target soft tissue of the subject (e.g., location of soft tissue defect, injury, and/or disease); and crosslinking (e.g., photo-crosslinking) the polymer composition by exposing the pre-gelation polymer composition to a crosslinking initiator (e.g., photoinitiator and visible light). In certain embodiments, the method includes removing the applicator from the gel polymer composition and/or soft tissue surface after the polymeric crosslinking and/or gelation of the polymer composition is complete. In certain embodiments, the pre-gelation polymer composition is applied directly to the surface of the target soft tissue without an applicator. In certain embodiments, the pre-gelation polymer composition is applied on or near (e.g., on the same tissue or under the tissue) the target soft tissue. In certain embodiments, the pre-gelation polymer composition can have a strong, sustained adhesion and high retention on the target soft tissue of the subject. In certain embodiments, the gel polymer composition can have a strong, sustained adhesion and high retention on the target soft tissue of the subject. In certain embodiments, the polymer composition is engineered to present physical, mechanical, structural, chemical and/or biological properties (elasticity, water content) to match or resemble the target soft tissue. In certain embodiments, the polymer composition is engineered to distribute a therapeutic agent to the target soft tissue.

### Ocular Injuries and Diseases

In certain embodiments, polymer compositions of the present disclosure can be used as a sealant and/or therapeutic composition for treating and/or repairing ocular soft tissue in the eye of a subject. In certain embodiments, polymer compositions of the present disclosure can be used as a sealant and/or therapeutic composition for treating and/or repairing an ocular defect, ocular surface injury, or an ocular disease in the eye of a subject. In certain embodiments, the ocular defect, injury or disease is a corneal or scleral defect, injury or disease. In certain embodiments, the corneal or scleral injury is a laceration (partial- or full-thickness), perforation, incision (e.g., surgical incision), or similar surface trauma (such as trauma from a foreign object or projectile). In certain embodiments, the ocular defect, injury or disease is an ocular ulcer, such as a corneal ulcer from severe infections, injuries, perforations, or other defects. In certain embodiments, the target soft tissue is ocular tissue; optionally subconjunctival ocular tissue or retinal ocular tissue.

In certain embodiments, the present disclosure provides methods for treating an ocular defect, ocular surface injury, or an ocular disease in a subject with the polymer compositions of the present disclosure. In certain embodiments, the method includes: applying a pre-gelation polymer composition of the present disclosure (e.g., a polymer composition comprising acryloyl-substituted gelatin) to an applicator; placing the applicator containing the pre-gelation polymer composition onto a surface of the eye of the subject; and crosslinking (e.g., photo-crosslinking) the polymer composition by exposing the pre-gelation polymer composition to a crosslinking initiator (e.g., visible light). In certain embodiments, the method includes removing the applicator from the gel polymer composition and/or ocular surface after the polymeric crosslinking and/or gelation of the polymer composition is complete. In certain embodiments, the pre-gelation polymer composition is applied directly to the surface of the target ocular tissue without an applicator. In certain embodiments, the pre-gelation polymer composition can have a strong, sustained adhesion and high retention on the ocular tissue of the subject. In certain embodiments, the gel polymer composition can have a strong, sustained adhesion and high retention on the ocular tissue of the subject. In certain embodiments, the polymer composition is engineered to present physical, mechanical, structural, chemical and/or biological properties (elasticity, water content) to match or resemble the target ocular tissue (e.g., corneal tissue).

In certain embodiments, the applicator is a curved, concave surface. In certain embodiments, the applicator is a curved lens (e.g., contact lens). In certain embodiments, the curvature of the applicator is similar to the curvature of the target ocular surface.

In certain embodiments, an ocular defect, ocular surface injury, or an ocular disease in a target ocular tissue can be treated by: (i) forming a pre-formed polymer composition by polymerizing a polymer composition of the present disclosure; and (ii) applying the pre-formed polymer composition onto a surface or under the surface (e.g., subconjunctival, subretinal) of the target tissue of the subject. In certain embodiments, application to the surface of a target tissue comprises application/injection to a space directly below the surface of a target tissue (e.g., subconjunctival application to ocular tissue, subretinal application to ocular tissue). In certain embodiments, the pre-formed polymer composition can be engineered to have specific physical, mechanical, structural, chemical and/or biological properties (e.g., elasticity, biodegradability, porosity).

In certain embodiments, an ocular defect, ocular surface injury, or an ocular disease in a target ocular tissue can be treated by: (i) forming a pre-formed hydrogel polymer composition by polymerizing a polymer composition of the present disclosure; (ii) drying the hydrogel polymer by removing a substantial portion of interstitial fluid from the hydrogel (e.g., at least 50%, at least 60%, at least 70%, at least 80%, at least 90%, or at least 95% of interstitial fluid); (iii) applying the pre-formed polymer composition onto a surface or under the surface (e.g., subconjunctival, subretinal) of the target tissue of the subject; and (iv) optionally rehydrating the dried hydrogel polymer to a substantially hydrated form (e.g., e.g., at least 50%, at least 60%, at least 70%, at least 80%, at least 90%, or at least 95% of interstitial fluid volume). In certain embodiments, application to the surface of a target tissue comprises application/injection to a space directly below the surface of a target tissue (e.g., subconjunctival application to ocular tissue, subretinal application to ocular tissue). In certain embodiments, the pre-formed polymer composition can be engineered to have specific physical, mechanical, structural, chemical and/or biological properties (e.g., elasticity, biodegradability, porosity).

### Oral Injuries and Diseases

In certain embodiments, polymer compositions of the present disclosure can be used as a sealant and/or therapeutic composition for treating and/or repairing soft tissue in the mouth of a subject. In certain embodiments, polymer compositions can be used for treating and/or repairing oral tissue associated with periodontal diseases, injuries or ailments. In certain embodiments, the periodontal disease, injury or ailment can include those associated with periodontal implants, including peri-implant diseases (PIDs) such as peri-implant mucositis (PIM) and peri-implantitis (PI). These ailments are often associated with inflammation (from bacterial accumulation and biofilm formation) of the soft tissues surrounding a periodontal implant, resulting in bleeding suppuration, eryfthema, swelling, and infection of the oral tissues, as well as possible progressive bone loss that can lead to implant failure.

In certain embodiments, polymer compositions of the present disclosure can be used to seal an area of soft tissue surrounding a periodontal implant. In certain embodiments, polymer compositions of the present disclosure can be used to deliver a therapeutic agent (e.g., antimicrobial or anti-inflammatory) to an area of soft tissue surrounding a periodontal implant. In certain embodiments, the polymer compositions comprises an osteoinductive agent. In certain embodiments, the polymer compositions comprises one or more osteoinductive agents selected from silicate nanoparticles (SNs), calcium salts, bioglass, hydroxyapatite, demineralized bone matrix (DBM), or combinations thereof. In certain embodiments, the polymer compositions comprises one or more silicate nanoparticles, including SNs that include one or more metals, such as calcium, aluminum, silver, gold, platinum, palladium, lithium, magnesium, sodium, titanium, vanadium, chromium, manganese, iron, cobalt, nickel, copper, zinc, iridium, or combinations thereof. In certain embodiment, the silicate nanoparticles include laponite nanoparticles. In certain embodiments, the polymer compositions comprises one or more calcium salts, such as calcium phosphate, calcium sulfate, calcium hydroxide, calcium bromide, calcium fluoride, calcium iodide, calcium hydride, or combinations thereof.

In certain embodiments, the present disclosure provides methods for treating a defect, injury, or disease in the oral soft tissue of a subject, with the polymer compositions of the present disclosure. In certain embodiments, the method includes: applying a pre-gelation polymer composition of the present disclosure (e.g., a polymer composition comprising acryloyl-substituted gelatin) to an applicator; placing the applicator containing the pre-gelation polymer composition onto a surface of the oral soft tissue of the subject (e.g., soft tissue surrounding a periodontal implant); and crosslinking (e.g., photo-crosslinking) the polymer composition by exposing the pre-gelation polymer composition to a crosslinking initiator (e.g., visible light). In certain embodiments, the method includes removing the applicator from the gel polymer composition and/or oral soft tissue surface after the polymeric crosslinking and/or gelation of the polymer composition is complete. In certain embodiments, the pre-gelation polymer composition is applied directly to the surface of the target oral soft tissue without an applicator. In certain embodiments, the pre-gelation polymer composition can have a strong, sustained adhesion and high retention on the oral soft tissue of the subject. In certain embodiments, the gel polymer composition can have a strong, sustained adhesion and high retention on the oral soft tissue of the subject. In certain embodiments, the polymer composition is engineered to present physical, mechanical, structural, chemical and/or biological properties (elasticity, water content) to match or resemble the target oral soft tissue (e.g., soft tissue surrounding a periodontal implant).

### Nerve Injuries and Diseases

In certain embodiments, polymer compositions of the present disclosure can be used as a sealant and/or therapeutic composition for treating and/or repairing soft tissue in the nervous system (e.g., central nervous system (CNS), peripheral nervous system (PNS)) of a subject. In certain embodiments, polymer compositions can be used for treating and/or repairing nerve tissue associated with traumatic injury or surgical damage, including Peripheral Nerve Injuries (PNI). Typical surgical interventions for these ailments (including suturing and/or commercial adhesives) are often associated with inflammation, heightened foreign body response (FBR), scaring, slower nerve regeneration, or loss of nerve function (partial or complete).

In certain embodiments, nerve tissue can be treated or sealed by applying a polymer composition of the present disclosure to the target nerve tissue. In certain embodiments, nerve tissue can be treated or sealed by applying a polymer composition of the present disclosure to the lumen of nerve conduits in the location of nerve injury.

In certain embodiments, the present disclosure provides methods for treating a defect, injury, or disease in the nerves or CNS tissue of a subject, with the polymer compositions of the present disclosure. In certain embodiments, the method includes: applying a pre-gelation polymer composition of the present disclosure (e.g., a polymer composition comprising acryloyl-substituted gelatin) to an applicator; placing the applicator containing the pre-gelation polymer composition onto a surface of the nerves or CNS tissue of the subject (e.g., nerves of the peripheral nervous system); and crosslinking (e.g., photo-crosslinking) the polymer composition by exposing the pre-gelation polymer composition to a crosslinking initiator (e.g., visible light). In certain embodiments, the method includes removing the applicator from the gel polymer composition and/or nerves/CNS tissue surface after the polymeric crosslinking and/or gelation of the polymer composition is complete. In certain embodiments, the pre-gelation polymer composition is applied directly to the surface of the target nerves or CNS tissue without an applicator. In certain embodiments, the pre-gelation polymer composition can have a strong, sustained adhesion and high retention on the target nerves or CNS tissue of the subject. In certain embodiments, the gel polymer composition can have a strong, sustained adhesion and high retention on the target nerves or CNS tissue of the subject. In certain embodiments, the polymer composition is engineered to present physical, mechanical, structural, chemical and/or biological properties (elasticity, water content) to match or resemble the target nerves or CNS tissue (e.g., nerves of the peripheral nervous system).

In certain embodiments, the polymer compositions of the present disclosure can include the polymeric or therapeutic components, or can be produced, analyzed or used by the methods (including for the treatment of nerve injuries) as disclosed in US 20190070338, which is incorporated herein by reference in its entirety, insofar as it describes the composition, production, analysis and use of acrylated gelatin polymeric compositions, such as GelMA hydrogels.

### Cardiovascular Injuries and Diseases

In certain embodiments, polymer compositions of the present disclosure can be used as a sealant and/or therapeutic composition for treating and/or repairing soft tissue in the cardiovascular system (e.g., heart) of a subject. In certain embodiments, polymer compositions can be used for treating and/or repairing cardiovascular tissue associated with traumatic injury or surgical damage, including cardiac tissue. Typical surgical interventions for these ailments (including suturing and/or commercial adhesives) are often associated with inflammation and infection, scaring, slower tissue regeneration, or loss of function (partial or complete).

In certain embodiments, vascular/cardiovascular tissue can be treated or sealed by applying a polymer composition of the present disclosure to the target vascular/cardiovascular tissue. In certain embodiments, vascular/cardiovascular tissue can be treated or sealed by applying a cell-laden hydrogel composition of the present disclosure to the target vascular/cardiovascular tissue. In certain embodiments, a cell-laden hydrogel composition comprises cells or cellular precursors which encourage or facilitate the repair, restoration, replacement, or regeneration of vascular/cardiovascular tissue (e.g., cardiac tissue). In certain embodiments, a cell-laden hydrogel composition comprises one or more cells or cellular precursors selected from: smooth muscle cells, cardiomyocytes, fibroblasts, mesenchymal stem cells, bone marrow stem cells, or a combination thereof. In certain embodiments, the cell-laden hydrogel composition is in the form of a mat, fabric, mesh, or other shape which is amenable to being used as a covering or transplant.

In certain embodiments, the present disclosure provides methods for treating a defect, injury, or disease in the cardiovascular tissue of a subject, with the polymer compositions of the present disclosure. In certain embodiments, the method includes: applying a pre-gelation polymer composition of the present disclosure (e.g., a polymer composition comprising acryloyl-substituted gelatin) to an applicator; placing the applicator containing the pre-gelation polymer composition onto a surface of the cardiovascular tissue of the subject (e.g., heart tissue); and crosslinking (e.g., photo-crosslinking) the polymer composition by exposing the pre-gelation polymer composition to a crosslinking initiator (e.g., visible light). In certain embodiments, the method includes removing the applicator from the gel polymer composition and/or cardiovascular tissue surface after the polymeric crosslinking and/or gelation of the polymer composition is complete. In certain embodiments, the pre-gelation polymer composition is applied directly to the surface of the target cardiovascular tissue without an applicator. In certain embodiments, the pre-gelation polymer composition can have a strong, sustained adhesion and high retention on the cardiovascular tissue of the subject. In certain embodiments, the gel polymer composition can have a strong, sustained adhesion and high retention on the cardiovascular tissue of the subject. In certain embodiments, the polymer composition is engineered to present physical, mechanical, structural, chemical and/or biological properties (elasticity, water content) to match or resemble the target cardiovascular tissue (e.g., heart tissue).

In certain embodiments, the polymer compositions of the present disclosure can include the polymeric or therapeutic components, or can be produced, analyzed or used by the methods (including for the treatment of cardiovascular injuries) as disclosed in WO2014063194, which is incorporated herein by reference in its entirety, insofar as it describes the composition, production, analysis and use of acrylated gelatin polymeric compositions, such as GelMA hydrogels.

### Lung Injuries and Diseases

In certain embodiments, polymer compositions of the present disclosure can be used as a sealant and/or therapeutic composition for treating and/or repairing soft tissue in the lungs of a subject. In certain embodiments, polymer compositions can be used for treating and/or repairing lung tissue associated with traumatic injury or surgical damage. Typical surgical interventions for these ailments (including suturing and/or commercial adhesives) are often associated with inflammation and infection, scaring, slower tissue regeneration, or loss of function (partial or complete).

In certain embodiments, lung tissue can be treated or sealed by applying a polymer composition of the present disclosure to the target lung tissue. In certain embodiments, lung tissue can be treated or sealed by applying a cell-laden hydrogel composition of the present disclosure to the target vascular/cardiovascular tissue. In certain embodiments, a cell-laden hydrogel composition comprises cells or cellular precursors which encourage or facilitate the repair, restoration, replacement, or regeneration of lung tissue. In certain embodiments, the cell-laden hydrogel composition is in the form of a mat, fabric, mesh, or other shape which is amenable to being used as a covering or transplant.

In certain embodiments, the polymer compositions comprises acryloyl-substituted gelatin (e.g., GelMA) and acryloyl-substituted PEG (e.g., PEGDA) at a ratio from about 30:1 to about 1:30 w/w. In certain embodiments, the polymer compositions comprises acryloyl-substituted gelatin (e.g., GelMA) and acryloyl-substituted Hyaluronic acid (e.g., MeHA) at a ratio from about 30:1 to about 1:30 w/w. In certain embodiments, the polymer compositions comprises acryloyl-substituted gelatin (e.g., GelMA), acryloyl-substituted PEG (e.g., PEGDA), and acryloyl-substituted Hyaluronic acid (e.g., MeHA).

In certain embodiments, the present disclosure provides methods for treating a defect, injury, or disease in the lung tissue of a subject, with the polymer compositions of the present disclosure. In certain embodiments, the method includes: applying a pre-gelation polymer composition of the present disclosure (e.g., a polymer composition comprising acryloyl-substituted gelatin) to an applicator; placing the applicator containing the pre-gelation polymer composition onto a surface of the lung tissue of the subject; and crosslinking (e.g., photo-crosslinking) the polymer composition by exposing the pre-gelation polymer composition to a crosslinking initiator (e.g., visible light). In certain embodiments, the method includes removing the applicator from the gel polymer composition and/or lung tissue surface after the polymeric crosslinking and/or gelation of the polymer composition is complete. In certain embodiments, the pre-gelation polymer composition is applied directly to the surface of the target lung tissue without an applicator. In certain embodiments, the pre-gelation polymer composition can have a strong, sustained adhesion and high retention on the lung tissue of the subject. In certain embodiments, the gel polymer composition can have a strong, sustained adhesion and high retention on the lung tissue of the subject. In certain embodiments, the polymer composition is engineered to present physical, mechanical, structural, chemical and/or biological properties (elasticity, water content) to match or resemble the target lung tissue.

### V. Definitions

At various places in the present disclosure, substituents, or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

GelMA polymer compositions: The term “GelMA polymer compositions” as used herein refers to.

Administering: As used herein, the term “administering” refers to providing a composition to a subject.

Amelioration: As used herein, the term “amelioration” or “ameliorating” refers to a lessening of severity of at least one indicator of a condition or disease.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Approximately: As used herein, the term “approximately” or “about,” as applied to one or more values of interest, refers to a value that is similar to a stated reference value. The term may refer to +/−10% of the recited value. In certain embodiments, the term refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Biocompatible: As used herein, the term “biocompatible” refers to a material which produces minimal or zero toxic, injurious, or immunological response in living tissue.

Biodegradable: As used herein, the term “biodegradable” refers to a material which can decompose partially or fully under physiological conditions into biologically-processable byproducts. For example, a material can be considered biodegradable if at least 10%, at least 20%, at least 30%, at least 40%, at least 50%, at least 60%, at least 70%, at least 80%, or at least 90% of the material can decompose under physiological conditions within a desired period of time (e.g., minutes, hours, days, weeks, or months, depending on the nature of the material and physiological application). The term “biodegradable” can encompass the term “bioresorbable,” which describes a substance that decomposes under physiological conditions, breaking down to products that undergo bioresorption into the host subject (e.g., as metabolites of biochemical systems).

Biologically active: As used herein, the term “biologically active” refers to a characteristic of any substance or material that has activity in a biological system and/or organism. For instance, a material that, when administered to an organism, has a biological effect on that organism, is considered to be biologically active.

Compound: Compounds of the present disclosure comprise all of the isotopes of the atoms occurring in the intermediate or final compounds. “Isotopes” refers to atoms having the same atomic number but different mass numbers resulting from a different number of neutrons in the nuclei. For example, isotopes of hydrogen comprise tritium and deuterium. The compounds and salts of the present disclosure can be prepared in combination with solvent or water molecules to form solvates and hydrates by routine methods.

Cross-link: As used herein, the terms “cross-link” or “cross-linking” refer bond formation (e.g. covalent bond formation) that links one polymer unit to another polymer unit.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point or native molecule.

Effective Amount: As used herein, the term “effective amount” of an agent is an amount sufficient to effect beneficial or desired results, for example, clinical results, and, as such, an effective amount depends upon the context in which it is being applied. For example, in the context of administering an agent that treats an ocular trauma or disorder, an effective amount of an agent is, for example, an amount sufficient to achieve treatment of the ocular trauma or disorder, as compared to the response obtained without administration of the agent.

Feature: As used herein, a “feature” refers to a characteristic, a property, or a distinctive element.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Modified: As used herein “modified” refers to a changed state or structure of a molecule of the present disclosure. Molecules may be modified in many ways comprising chemically, structurally, and functionally. As used herein, embodiments of the disclosure are modified when they have or possess a feature or property, whether structural or chemical, that varies from a starting point or native molecule.

Non-human animal: As used herein, a “non-human animal” includes all animals (e.g., vertebrates) except Homo sapiens, including wild and domesticated species. Examples of non-human vertebrate animals include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak. Non-human animals include non-human primates.

Pharmaceutically acceptable: The terms “pharmaceutically acceptable” or “therapeutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The terms “pharmaceutically acceptable excipient” or “therapeutically acceptable excipient”, as used herein, refer to an ingredient other than the polymeric compositions described herein (e.g., a vehicle capable of suspending or dissolving the polymeric compound) and having the properties of being substantially nontoxic and non-inflammatory in a subject.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts comprise, but are not limited to, mineral or organic acid salts of basic residues such as amines; alkali or organic salts of acidic residues such as carboxylic acids; and the like. Representative acid addition salts comprise acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, 4-(2-hydroxyethyl)-1-piperazineethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts comprise sodium, lithium, potassium, calcium, magnesium, and the like, as well as nontoxic ammonium, quaternary ammonium, and amine cations, comprising, but not limited to ammonium, tetramethylammonium, tetraethylammonium, methylamine, dimethylamine, trimethylamine, triethylamine, ethylamine, and the like. The pharmaceutically acceptable salts of the present disclosure comprise the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used.

Subject: As used herein, the term “subject” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the arts will understand that biological and chemical phenomena rarely, if ever, go to completion and/or proceed to completeness or achieve or avoid an absolute result. The term “substantially” is therefore used herein to expressly capture the potential lack of completeness inherent in many biological and chemical phenomena. Likewise, the exclusion of the term “substantially” does not preclude the same potential lack of completeness inherent in many biological and chemical phenomena.

Synthetic: The term “synthetic” means produced, prepared, and/or manufactured by the hand of man. Synthesis of polynucleotides or polypeptides or other molecules of the present disclosure may be chemical or enzymatic.

Therapeutic Agent: The term “therapeutic agent” refers to any agent that, when administered to a subject, has a therapeutic, diagnostic, and/or prophylactic effect and/or elicits a desired biological and/or pharmacological effect. Examples of therapeutic agents include, but are not limited to: oligonucleotides (e.g., sense and/or antisense DNA and/or RNA), proteins and polypeptides (e.g., hormones, growth factors), small molecules and pharmaceuticals, and cells (e.g., stem cells, epithelium cells).

Treating: As used herein, the term “treating” refers to partially or completely alleviating, ameliorating, improving, relieving, preventing, delaying onset of: inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Those skilled in the art will recognize or be able to ascertain using no more than routine experimentation, many equivalents to the specific embodiments in accordance with the present disclosure described herein. The scope of the present disclosure is not intended to be limited to the above Description.

In the claims, articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure can include embodiments in which exactly one member of the group is present in, employed in, or otherwise relevant to a given product or process. The present disclosure can include embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

The abbreviation, “e.g.,” is derived from the Latin exempli gratia, and is used herein to indicate a non-limiting example. Thus, the abbreviation “e.g.,” is synonymous with the term “for example”.

The abbreviation, “i.e.,” is derived from the Latin id est, and is used herein to indicate a non-limiting rewording or clarification. Thus, the abbreviation “i.e.,” is synonymous with the term “that is”.

Where ranges are given, endpoints are comprised. Furthermore, it is to be understood that unless otherwise indicated or otherwise evident from the context of the disclosure and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, comprising definitions, will control. In addition, section headings, the materials, methods, and examples are illustrative only and not intended to be limiting.

### EXAMPLES

### Example 1. Preparation of Precursor Polymeric Compositions

### (a) Preparation of Gelatin Methacryloyl (GelMA) Precursor Polymeric Composition

GelMA precursor polymeric compositions can be synthesized as described in the art. For example, GelMA is synthesized by dissolving 10% (w/v) gelatin (e.g., porcine gelatin) in phosphate-buffered saline (PBS), and then heated at 60° C. for 20 minutes. The heating is followed by dropwise addition of 8% (v/v) methacrylic anhydride at 50° C. for 3 hours (under continuous stirring), followed by dilution with PBS and dialysis at 40-50° C. for about 7 days (using deionized water). The resulting mixture is filtered and lyophilized for 4 days. The resulting GelMA precursor polymeric composition can be stored at −80° C. until further use.

In one alternative, GelMA is synthesized by dissolving 10 grams of gelatin from fish skin in 100 ml DPBS at 60° C. for 30 minutes. 8% (v/v) methacrylic anhydride is then added to the solution drop-wise under stirring at 60° C. for 3 hours. An additional 300 ml DPBS is added to halt the reaction. The resulting mixture is dialyzed using a deionized water bath at 50° C. for about 5 days to remove the unreacted methacrylic anhydride. The resulting solution is filtered and lyophilized for about 4 days.

### (b) Preparation of Methacrylated Hyaluronic Acid (MeHA) Precursor Polymeric Composition

MeHA precursor polymeric compositions can be synthesized as described in the art, such as those presented in: Bencherif et al., Biomaterials 29, 1739-1749 (2008); Prata et al., Biomacromolecules 11, 769-775 (2010). For example, MeHA is synthesized by dissolving about 2 grams of hyaluronic acid sodium salt in 200 ml of deionized water, followed by the sequential addition of 8.0 mL triethylamine, 8.0 mL glycidyl methacrylate, and 4.0 g of tetrabutyl ammonium bromide (with 1 hour of stirring between each sequential addition). The resulting mixture is incubated at 55° C. for 1 hour, then cooled (ice bath) and precipitated in acetone (4 L) to form a white solid precipitate. The precipitate is rinsed with fresh acetone, dissolved in pure water, dialyzed for 2 days, then frozen and lyophilized for storage.

### (c) Preparation of Polyethylene Glycol Diacrylate (PEGDA) Precursor Polymeric Composition

PEGDA precursor polymeric compositions can be synthesized as described in the art. For example, PEGDA is synthesized by reacting 10 grams of PEG in dichloromethane (10% w/v) with triethylamine and acryloyl chloride (1:4:4 molar ratio) at 4° C. under inert conditions (stirred overnight). The resulting mixture is filtered and then precipitated using ice-cold ether. The resulting precipitated product is filtered and dried in vacuum desiccator overnight to remove residual materials.

In one alternative, PEGDA is synthesized by dissolving PEG diol in benzene, followed by azeotropic distillation in toluene using a Dean-Stark trap to remove water and ensure dry acrylation conditions. PEG acrylation is carried out by dissolving PEG in dichloromethane solution (under argon), followed by the addition of acryloyl chloride and triethylamine at a molar ratio of 2:3:3 of OH-groups of PEG:acryloyl chloride:triethylamine. The resulting mixtures stirred at room temperature (dark room conditions) overnight. The resulting product is then precipitated using diethyl ether and chilled to 4° C., followed by filtration recovery and vacuum oven drying.

### Example 2: Preparation of Hydrogel Polymeric Composition

Hydrogel polymeric compositions can be synthesized as described in the art. For Example, a freeze-dried GelMA precursor polymeric composition produced according to Example 1(a) is dissolved in PBS or (4-(2-hydroxyethyl)-1-piperazineethanesulfonic acid) buffered saline at concentrations of 5-25% (w/v). Either 2-hydroxy-4′-(2-hydroxyethoxy)-2-methylpropiophenone or Eosin Y or Eosin Y disodium salt is added as a photoinitiator, and the mixture is dissolved at 20-80° C. The resulting precursor polymeric composition is photocrosslinked via visible light irradiation (e.g., blue or white light) to form a GelMA hydrogel polymeric composition. In one alternative, a target concentration of MeHA precursor polymeric composition [Example 1(b)], and/or PEGDA precursor polymeric composition [Example 1(c)] can be added to the precursor polymeric solution, wherein the amount of each element is added based on the desired physical, mechanical, structural, chemical and/or biological properties of the Hydrogel polymeric composition.

In one alternative, a GelMA hydrogel polymeric composition is synthesized by first dissolving 7-15% w/v of gelatin methacryloyl from Example 1 into a solution containing at least one photoinitiator element, such as a mixture of triethanolamine (about 2% w/v) and N-vinyl caprolactam (about 1.25% w/v), in distilled water at room temperature. A solution of Eosin Y disodium salt (0.5 mM) is then added to the gelatin methacryloyl solution, and the resulting precursor polymeric composition is then photocrosslinked under exposure to visible light (420-480 nm) for 120 seconds. In one alternative, a target concentration of MeHA precursor polymeric composition [Example 1(b)], and/or PEGDA precursor polymeric composition [Example 1(c)] can be added to the precursor polymeric solution, wherein the amount of each element is added based on the desired physical, mechanical, structural, chemical and/or biological properties of the Hydrogel polymeric composition.

In one alternative, microparticles (e.g., micelles) which contain a therapeutic agent (e.g., ocular antibiotic such as ciprofloxacin) are incorporated into the GelMA precursor polymeric composition prior to photocrosslinking.

Porosity can be measured and analyzed by fabricating a freeze-dried, gold-sputter-coated hydrogel sample, which can then be imaged using a scanning electron microscope (SEM).

Samples can also be subjected to a range of mechanical tests, including elasticity, swelling, compression testing, texture, and tensile testing.

In one alternative, a GelMA hydrogel polymeric composition is formed on the surface of a target tissue. Resulting samples can be subjected to a range of mechanical and therapeutic tests, including adhesion, burst pressure, wound closure strength, shear strength, and durability/degradation rate.

### Example 3: Preparation of Hydrogel Polymeric Composition

Hydrogel polymeric compositions were prepared according to the follow steps.

A photopolymerization initiator mixture was prepared comprising: 0.35 mg/mL of eosin Y (20% v/v), 12.5 mg/mL N-vinylcaprolactam, and 18.75 mg/mL triethanolamine (80% v/v), in phosphate buffer saline (PBS; pH 7), with pH adjustment using concentrated HCl as needed.

Polymeric precursors were obtained from the following sources: (1) GelMA—Rousselot Biomedical (160P80 or GelMA 160P40 or GelMA 160P10); (2) HAMA—HTL Biotechnology (BLo-RD029-008); (3) HAGM—synthesized in-house according to methods known in the art (See, e.g., Example 1(b)); and (4) PEGDA—Jen Kem (ACLT-PEG35K-ACLT). Polymeric precursors were allowed to reach room temperature (RT) before their incorporation into a hydrogel polymeric precursor composition.

PEGDA precursor materials (when applicable for a target formulation) were added first at the desired concentration (e.g., 0.1-20% w/v) into the photopolymerization initiator mixture, and allowed to dissolve at 37° C. for about 5 minutes.

GelMA precursor materials (when applicable for a target formulation) were then added at the desired concentration (e.g., 4-20% w/v) into the hydrogel precursor mixture, and allowed to dissolve at 60° C. for about 2 hours with occasional vortexing.

MeHA (i.e., HAMA or HAGM) precursor materials (when applicable for a target formulation) were then added at the desired concentration (e.g., 1-3% w/v) into the hydrogel precursor mixture, and allowed to dissolve at 60° C. overnight with stirring (to prevent any phase separation).

Once all precursor materials were fully dissolved into the hydrogel precursor mixture, an active agent (when applicable for a target formulation) was added at the desired concentration (e.g., 1-350 mg/mL). The mixture was maintained under stirring at 37° C. until ready for polymerization.

Hydrogel disk samples were prepared by pipetting about 100 μL of hydrogel precursor mixture into individual poly(dimethylsiloxane) (PDMS) cylindrical molds positioned in wells of a 24-well non-treated plate. The polymer composition was then photocrosslinked using a Dolan-Jenner high-intensity LED illuminator (MI-LED-US-B1) equipped with a dual-arm gooseneck configuration (one arm above and one arm below, thus allowing for dual light exposure from top and bottom; incident ray 90°). Each arm outputs an average optical power of ˜100 mW/cm2 (λmax=450, 540 nm), with light exposure times varying from 5 seconds to 4 minutes.

Hydrogel rod samples were prepared by dipping 0.75 mm inner-diameter borosilicate glass capillaries into the hydrogel precursor mixture, and then oscillating the capillary tubes until filled up to about 10 mm from the opening. The polymer composition was then photocrosslinked using a Dolan-Jenner high-intensity LED illuminator (MI-LED-US-B1) equipped with a dual-arm gooseneck configuration (one arm above and one arm below, thus allowing for dual light exposure from top and bottom; incident ray 90°). Each arm outputs an average optical power of ˜100 mW/cm2 (λmax=450, 540 nm), with light exposure times of about 4 minutes. Hydrogel rods were extruded from the capillary tubes using a 0.5 mm diameter quartz rod, and then cut to size using calipers.

### Example 4: Study of Hydrogel Properties

### A) Degree of Crosslinking—Photopolymerization Time

Studies were completed to analyze the correlation between the degree of crosslinking within hydrogels as a function of photopolymerization time.

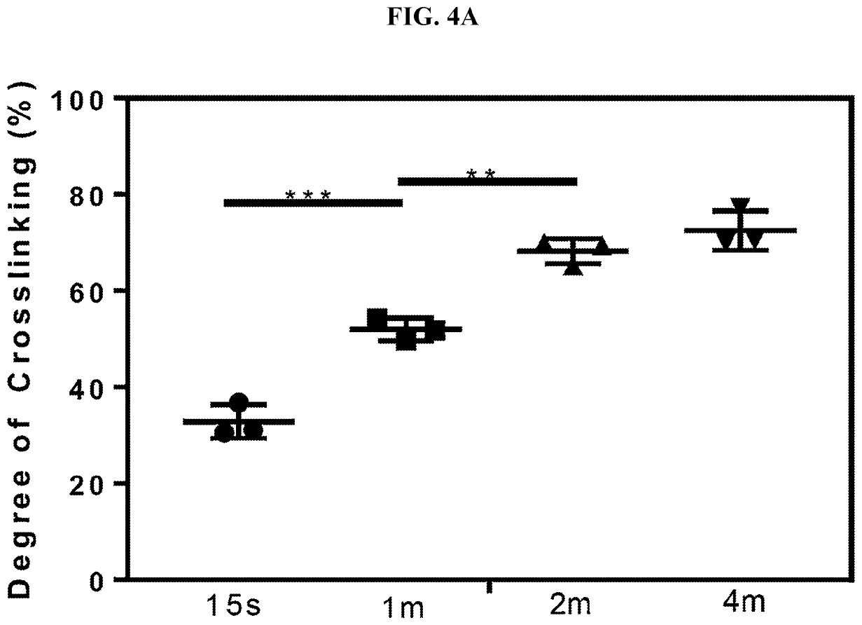

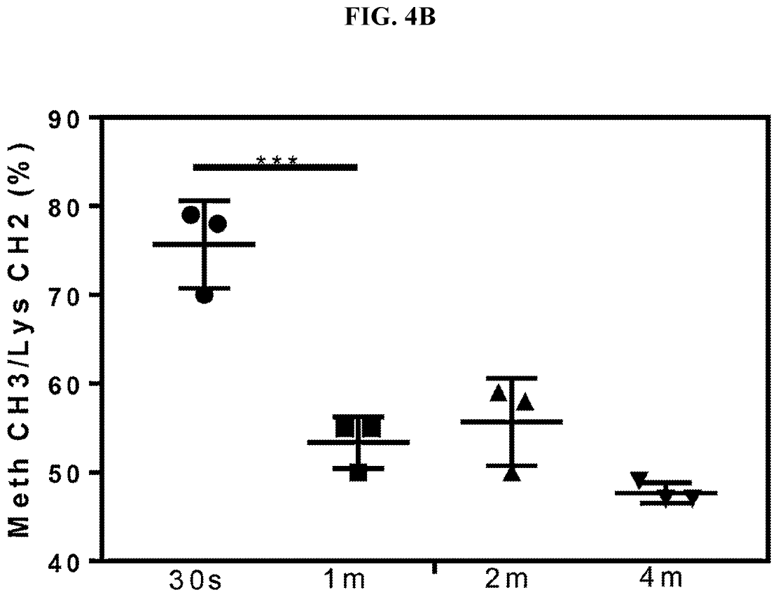

HAMA—only hydrogels were prepared according to the general procedures of Example 3, with photocrosslinking times of 15 seconds, 1 minute, 2 minutes, and 4 minutes. The resulting hydrogels were dried under vacuum, dissolved in deuterated DMSO, and then analyzed using proton NMR analysis (d-DMSO solvent). Other techniques can also be used, such as Fourier-transform infrared spectroscopy (FTIR) and Raman spectroscopy. For HAMA hydrogels, the change in proton ratio between the methacrylate methyl group and the HA carbonyl methyl group was quantified as a function of light exposure time and normalized to the ratio present in uncrosslinked HAMA to represent the degree (%) of crosslinking. Results in FIG. 4A show that degree of crosslinking increases as light exposure time increases.

GelMA-only hydrogels were prepared according to the general procedures of Example 3, with photocrosslinking times of 30 seconds, 1 minute, 2 minutes, and 4 minutes. The resulting hydrogels were dried under vacuum, dissolved in deuterated DMSO, and then analyzed using proton NMR analysis (d-DMSO solvent). Other techniques can also be used, such as Fourier-transform infrared spectroscopy (FTIR) and Raman spectroscopy. For GelMA hydrogels, the ratio of ME methyl groups to GelMA lysine CH₂ groups was analyzed. Results in FIG. 4B show that ratios of [ME methyl groups to lysine CH₂ groups] decrease as light exposure time increases.

### b) Swelling Ratio

Studies were completed to analyze the swelling ratios of hydrogels having various GelMA, HAMA, and PEGDA concentrations.

G4-H_{M}1-P1, G7-H_{M}1, G4-H_{M}1, and HMI-P1 hydrogels (as described in Table 1) were prepared according to the general procedures of Example 3, with photocrosslinking times of 4 minutes. Resulting hydrogel cylinders had diameter of 6 mm and a volume of 75 IL.

To assess swelling, two methods were employed. In the first method, hydrogel weight right after crosslinking was used as the “dry” hydrogel weight (Wd-1) while in the second method the dry polymer weight (hydrogels dried in vacuo) was used as the dry hydrogel weight (Wd-2). In both instances the “wet” hydrogel weight (Ws) referred to hydrogels incubated at 37° C. in 1×PBS for 48 hours. Swelling ratio was calculated as follows:

$$Swelling Ratio = ( Ws - Wd ) / Wd$$

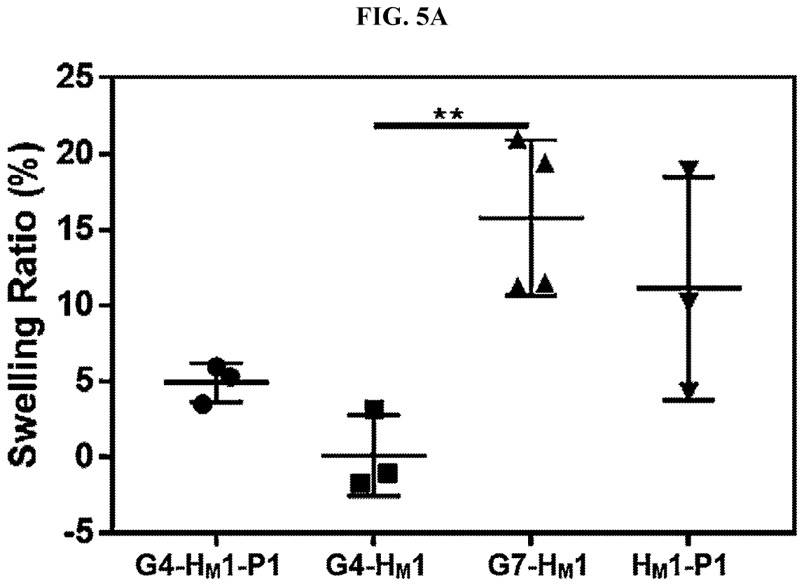

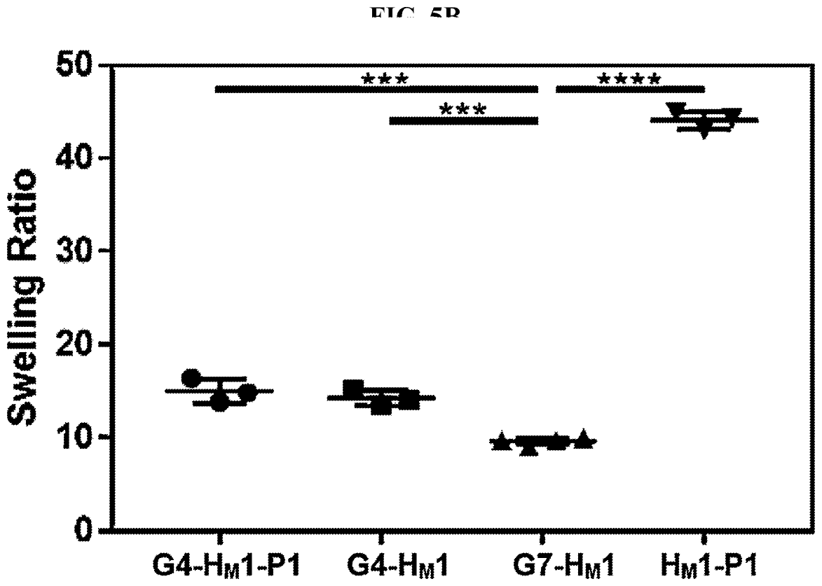

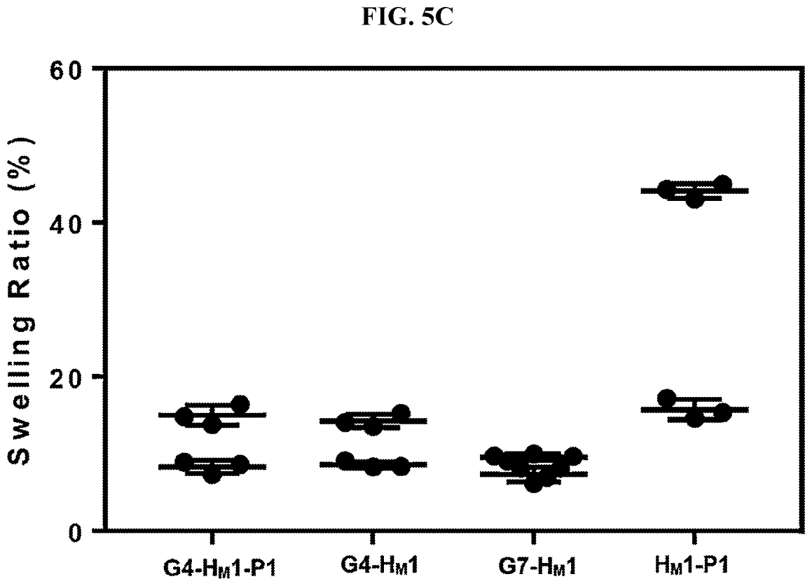

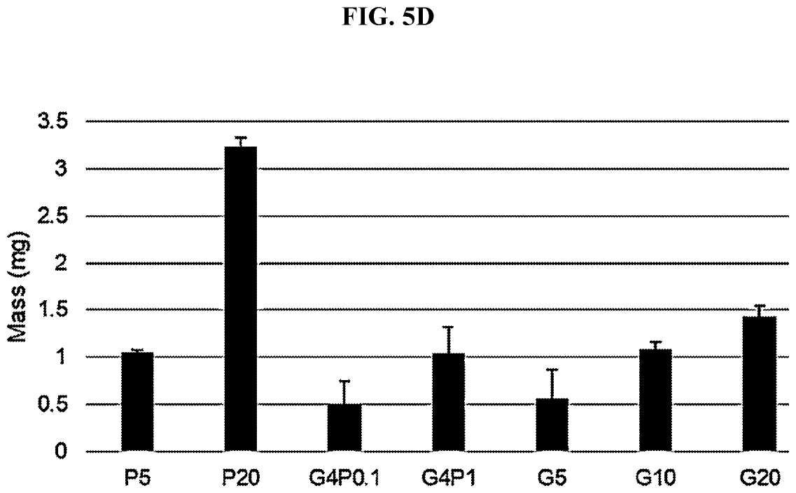

Results from the First measurement method were inconsistent, as shown in FIG. 5A. Results from the Second measurement method were more consistent, as shown in FIG. 5B, and shows that increased GelMA concentration plays a role in reducing the hydrogel swelling.

G4-H_{M}1-P1. G7-H_{M}1, G4-H_{M}1, and H_{M}1-P1 hydrogels were studied for swelling/reswelling effects. Samples were dried and swelled using the second method, and then re-dried and re-swelled a second time. Results presented in FIG. 5C show that swelling ratios are notably decreased when they hydrogel is exposed to more than one drying/swelling cycle.

G4-P1, G4-P0.1, G20, G10, G5, P20, and P5 hydrogels (as described in Table 1) were prepared according to the general procedures of Example 3, with photocrosslinking times of 4 minutes. Swelling was assessed using a dry polymer weight (hydrogels dried in vacuo) as the “dry” hydrogel weight (Wd), and with the “wet” hydrogel weight (Ws) referring to hydrogels incubated at 37° C. in 1×PBS for 48 hours. Results presented in FIG. 5D show that swelling mass are notably increased with the inclusion of PEGDA, and that increased GelMA concentrations also increase the swelling mass of the hydrogel.

C) Swelling Ratio with Active Agent

Studies were completed to analyze the swelling ratios of hydrogels loaded with an active agent and having various GelMA, HAMA, and PEGDA concentrations.

G4-H_{M}1-P1, G4-H_{M}1, G7-H_{M}1, H_{M}1-P1, G4-P1, and G7-P1 hydrogels (as described in Table 1) according to the general procedures of Example 3, with photocrosslinking times of 4 minutes. Samples of each hydrogel were also prepared with 13.2 mg/mL of a corticosteroid active agent.

Swelling was assessed using a dry polymer weight (hydrogels dried in vacuo) as the “dry” hydrogel weight (Wd), and with the “wet” hydrogel weight (Ws) referring to hydrogels incubated at 37° C. in 1×PBS for 48 hours. Swelling ratio was calculated as follows:

$$Swelling Ratio = ( Ws - Wd ) / Wd$$

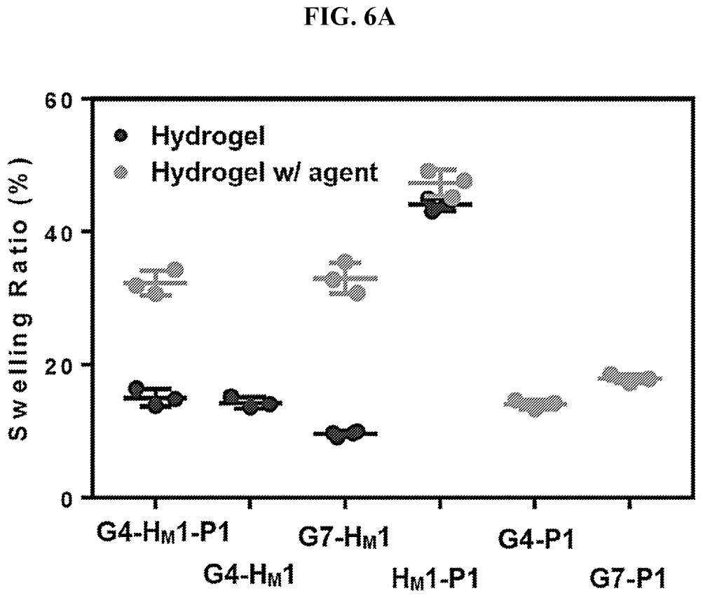

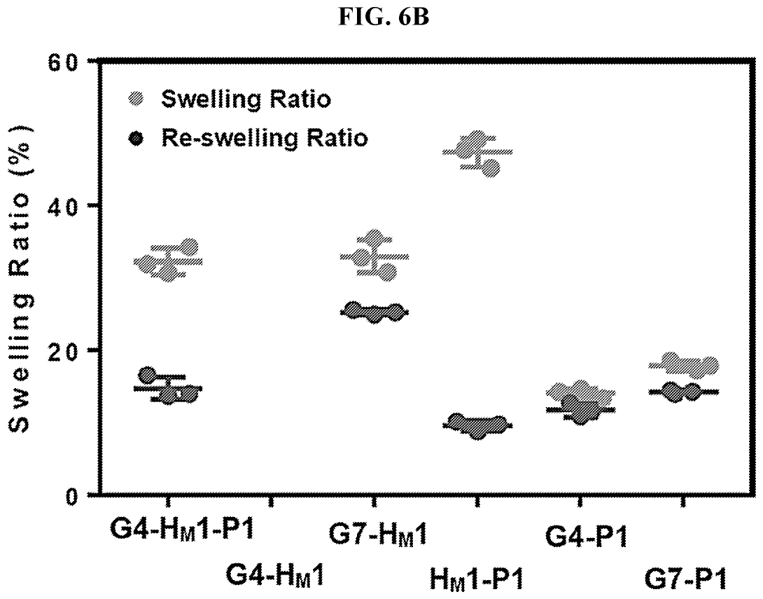

Results presented in FIG. 6A show hydrogels loaded with an active agent generally have a higher swelling ratio, likely due to the gel-network crosslinking disruptions and lower crosslinking density associated with incorporating an active agent into the gel network.

G4-H_{M}1-P1, G4-H_{M}1, G7-H_{M}1, H_{M}1-P1, G4-P1, and G7-P1 hydrogels (with active agents) were also studied for swelling/reswelling effects. Samples were dried and swelled, and then re-dried and re-swelled a second time. Results presented in FIG. 6B show that swelling ratios for hydrogels containing MeHA are notably decreased when the hydrogel is exposed to more than one drying/swelling cycle, while hydrogels that contain only GelMA+PEGDA have minimal effect from re-swelling.

### d) Enzymatic Degradation

Studies were completed to analyze enzymatic degradation stabilities of hydrogels having various GelMA, MeHA, and PEGDA concentrations.

G4-H_{G}3-P1, G4-H_{M}1-P0.67, G4-H_{G}3, G4-H_{M}1, G7-H_{G}3, G7-H_{M}1, H_{G}3-P1, and H_{M}1-P0.67 hydrogels (as described in Table 1) were prepared according to the general procedures of Example 3, with photocrosslinking times of 4 minutes. Samples were then enzymatically digested in hyaluronidase (Hy) and either Collagenase Type I (C_{I}) or Collagenase Type II (C_{II}) at either 20 U/mL or 2 U/mL. Resulting degradation times are shown in Table 2.

TABLE 2
Enzymatic Degradation Times

| | | 2 U/mL | 2 U/mL |
|---|---|---|---|
| Formulation | 20 U/mL | Hy + C_{I} | Hy + C_{I} |
| G4-H_{G}3-P1 | 1-2 days | 32 days | — |
| G4- H_{M}1-P0.67 | | — | 6 days |
| G7-H_{G}3 | | 6 days | — |
| G7-H_{M}1 | | — | 6 days |
| G4-H_{G}3 | | — | — |
| G4-H_{M}1 | | — | 7 days |
| H_{G}3-P1 | | 14 days | — |
| H_{M}1-P0.67 | | — | 12 days |

### e) Drug Release

Studies were completed to analyze drug release rate of hydrogels having various GelMA, MeHA, and PEGDA concentrations.

G4-H_{M}1-P1 and G4-H_{G}3-P1 hydrogels (as described in Table 1) were prepared with 13.2 mg/mL of a corticosteroid active agent according to the general procedures of Example 3, with photocrosslinking times of 4 minutes. Resulting hydrogel cylinders had diameter of 6 mm and a volume of 75 μL.

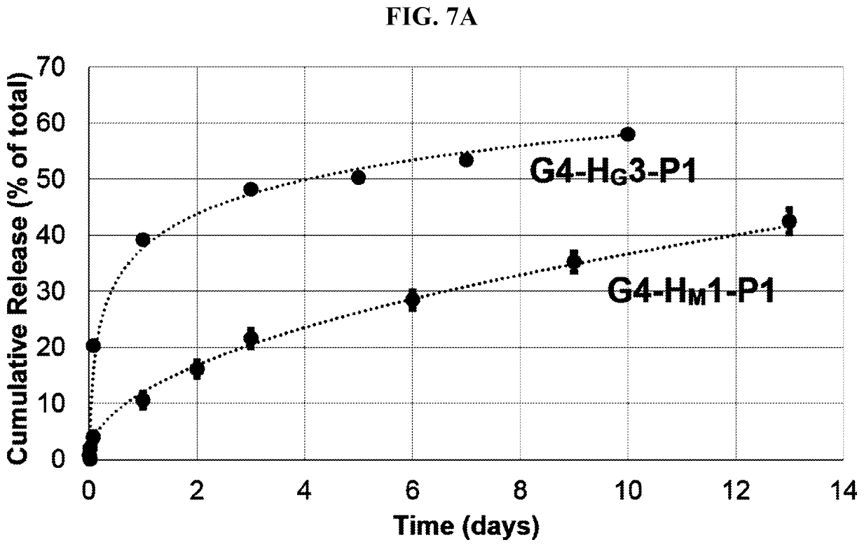

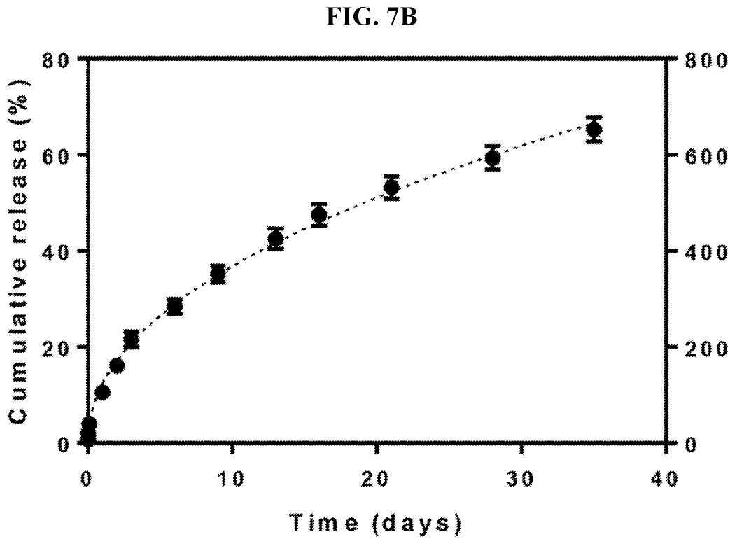

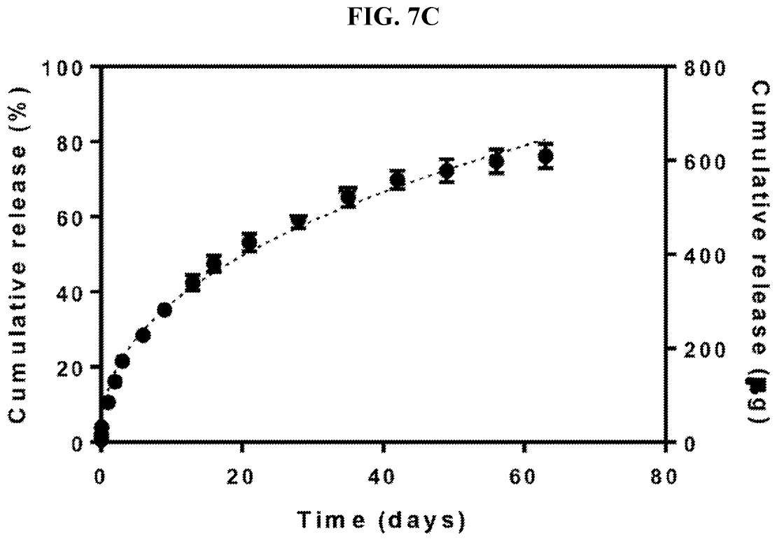

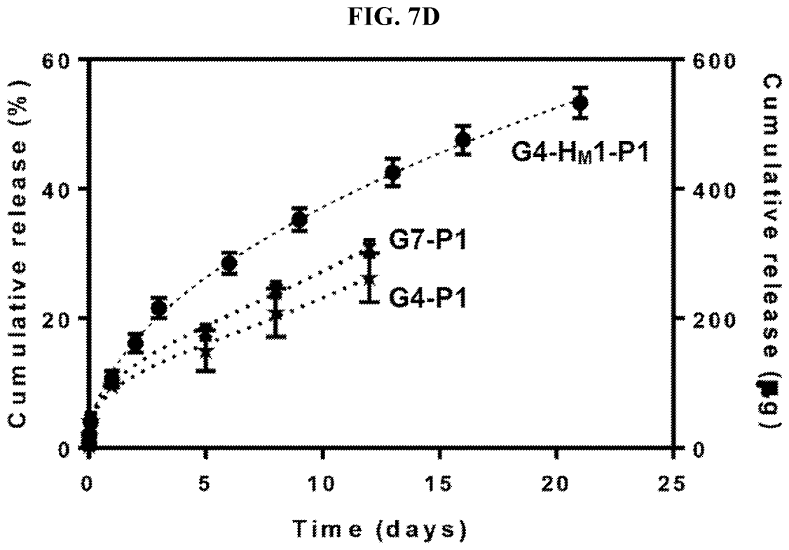

For release studies, hydrogels were statically (no physical agitation) incubated at 37° C. in 1 mL of 1×PBS supplemented with 2% Triton X-100 to simulate tear fluid. At each time point (over 10-13 days), the incubation solution was completely removed and replaced with fresh 1×PBS+2% Triton X-100. In order to quantify corticosteroid release, samples were diluted 1:2 in acetonitrile and analyzed using reverse phase liquid chromatography. An Agilent Zorbax Eclipse (XDB-C18) 4.6×250 mm, 5 μm analytical column was used on an Agilent 1290 HPLC system equipped with a diode array detector. The column was equilibrated at 70% acetonitrile, 30% water at 25° C. After injecting a 20 μL sample, the solvent gradient increased from 70% to 90% ACN during the time-span of 10 minutes. The corticosteroid eluted close to 5 minutes when the ACN gradient reaches approximately 80%. This peak was integrated and the area under the curve was used to determine concentration by comparing it to a standard curve for the corticosteroid. Results presented in FIG. 7A show that hydrogels containing a higher concentration of MeHA provide a more accelerated release profile. These results correlate with corresponding study results showing that higher concentrations of MeHA in a hydrogel result in increased hydrogel swelling.

Based on results in the Swelling Ratio studies, it is likely that higher concentrations of MeHA in the hydrogel result in increased hydrogel swelling, and thus resulting in a more accelerated burst release of the active agent. Higher concentrations of MeHA may also lead to a phase separation with GelMA within the precursor solution, which can cause gel network imperfections (i.e., areas of higher and lower crosslinking density) resulting in a higher initial burst release.

Release profiles for G4-H_{M}1-PI were continued through 35 days (FIG. 7B) and through 65 days (FIG. 7C). Release profile for G4-H_{M}1-P1 was also compared with G4-P1 and G7-P1 (FIG. 7D), again showing that the presence of MeHA in a hydrogel increases the release rate of the active agent from the hydrogel.

### f) Vacuum Drying

Studies were completed to analyze the effect of vacuum drying on the drug release rate of hydrogels having various GelMA, MeHA, and PEGDA concentrations.

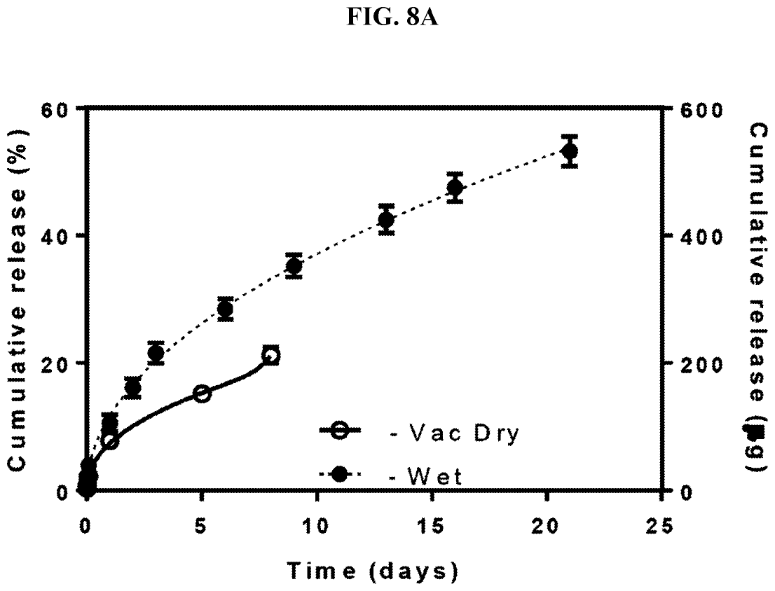

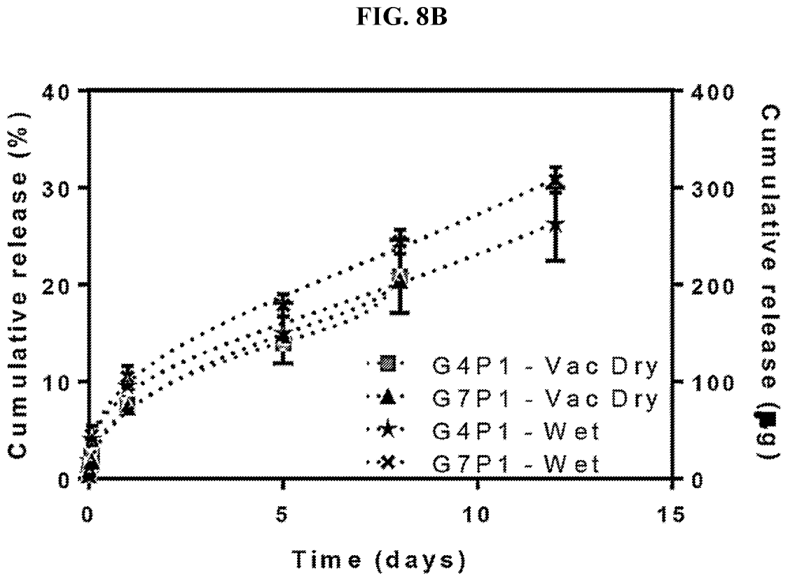

G4-H_{M}1-P1, G4-P1, and G7-P1 hydrogels (as described in Table 1) were prepared with 13.2 mg/mL of a corticosteroid active agent according to the general procedures of Example 3, with photocrosslinking times of 4 minutes. Samples from each hydrogel were then vacuum dried. Release studies using wet and dried samples for each hydrogel where then completed according to the general study procedures of Example 3(e). Results for G4-HMT-P1 hydrogels (FIG. 8A) show that release profiles for hydrogels containing MeHA can be reduced by vacuum drying the hydrogel, such that the inclusion of MeHA in a hydrogel formulation can reduce the release profile for dried samples, while alternatively increasing the swelling and corresponding release profiles in samples that would not be dried. Results for G4-P1 and G7-P1 hydrogels (FIG. 8B) show that release profiles for GelMA+PEGDA hydrogels that do not contain MeHA are generally not affected by vacuum drying the hydrogel.

2) Rods Vs. Disks

Studies were completed to analyze the effect of hydrogel shape (i.e., rods vs. disks) on the drug release rate of hydrogels comprising GelMA, MeHA, and PEGDA.

G4-HMI-P1 hydrogels (as described in Table 1) were prepared with 13.2 mg/mL of a corticosteroid active agent as both disks and rods, according to the general procedures of Example 3 and with photocrosslinking times of 4 minutes.

G4-H_{M}1-P1 hydrogel disks had a diameter (D) of 6 mm, a volume (V) of 75 μL, a surface area (SA) of 107 mm², and a SA:V ratio of 1.4.

G4-H_{M}1-P1 hydrogel rods had a diameter (D) of 2 mm, a volume (V) of 25 μL, a surface area (SA) of 56 mm², and a SA:V ratio of 2.2.

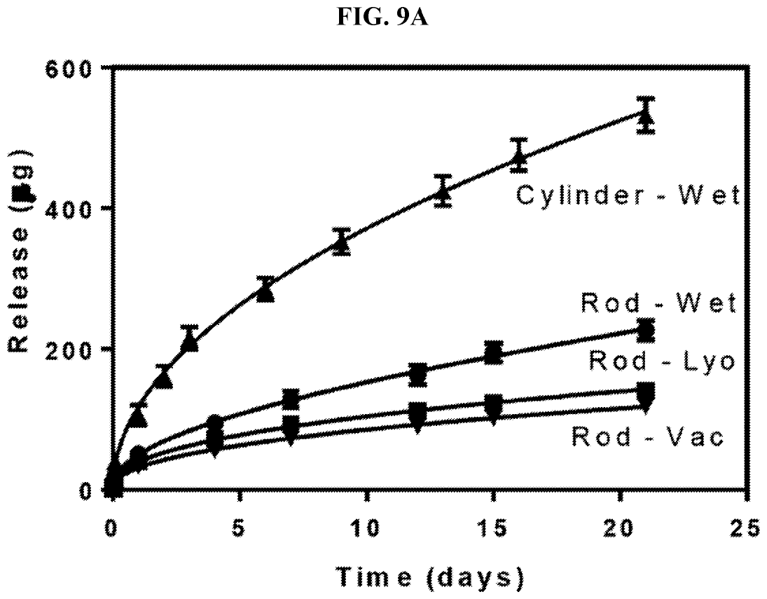

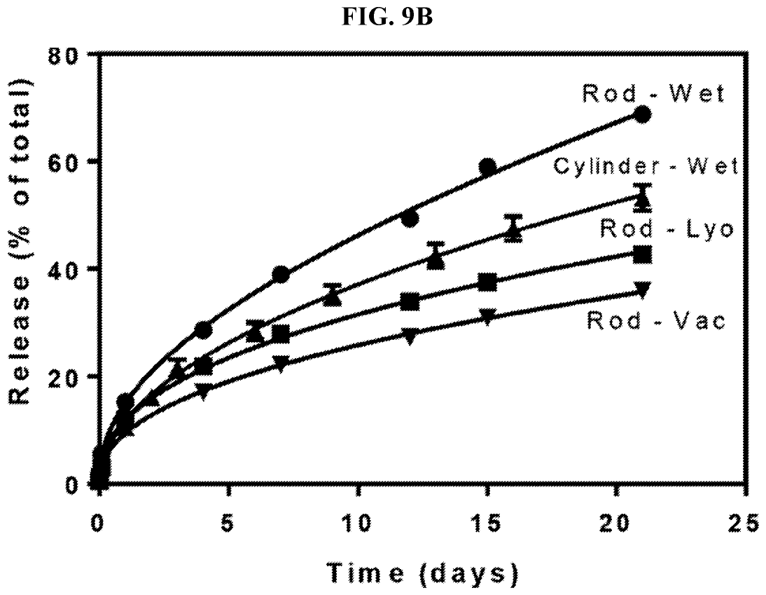

Samples from the rod hydrogels were then vacuum dried or freeze dried (i.e., lyophilized). Release studies using the resulting wet and dried samples where then completed according to the general study procedures of Example 3(e). Results for Total Drug Release (FIG. 9A) show that cylinder disks provide a larger total release of the active agent (likely as a result of a high surface area), with Rod_{wet}, Rod_{lyo}, and Rod_{dry} all having similar release totals. Results for Percentage Drug Release (FIG. 9B) show wet hydrogels (cylinder disk and rods) release a higher percentage of active agent than vacuum dried or freeze-dried rod hydrogels. Study results thus showed that the swelling properties, surface area (i.e., shape), and hydration state of a hydrogel play a role in the drug release profile of a hydrogel composition.

### H) Degree of Crosslinking—Degree of Methacrylation

Studies were completed to analyze the correlation between the release profile of a GelMA+PEGDA hydrogel and the degree of GelMA methacrylation within the hydrogels.

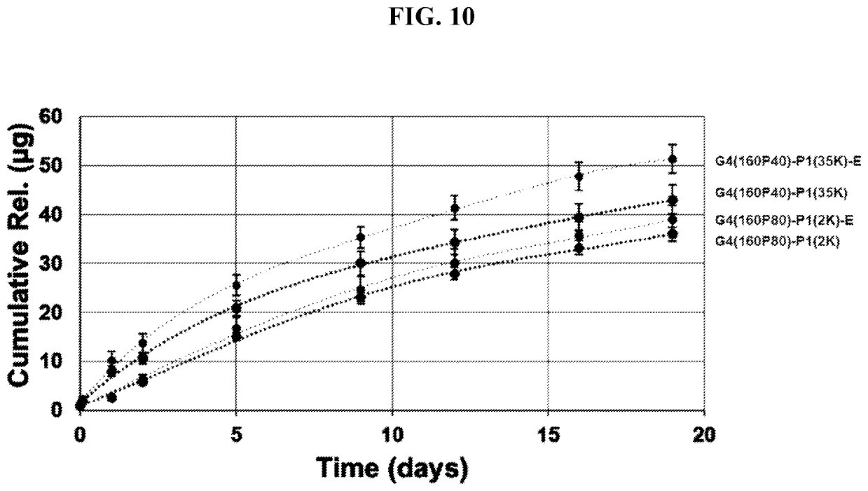

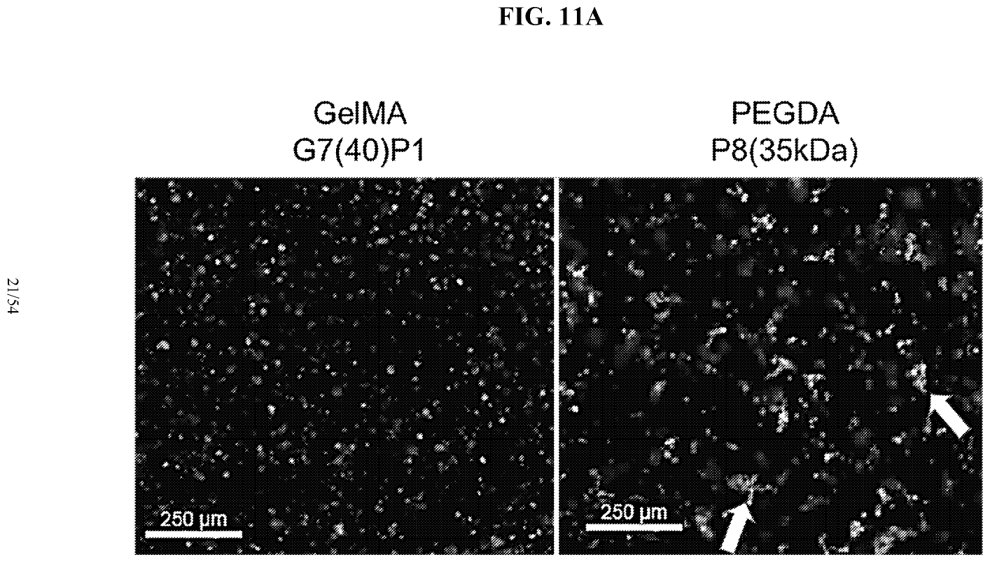

G4(160P80)-P1(2K) and G4(160P40)-P1(35K) hydrogels (as described in Table 1) were prepared with 13.2 mg/mL of a corticosteroid active agent according to the general procedures of Example 3 and with photocrosslinking times of 4 minutes. Release studies where then completed according to the general study procedures of Example 3(e), with each sample being exposed to Collagenase II 0.5 U/mL conditions and non-enzymatic standard conditions. Results for Total Drug Release (FIG. 10) show that a lower 40% DoM in the GelMA provide a faster release profile than the higher 80% DoM GelMA hydrogel.

### Example 5: Cellular Aggregation and Viability Study

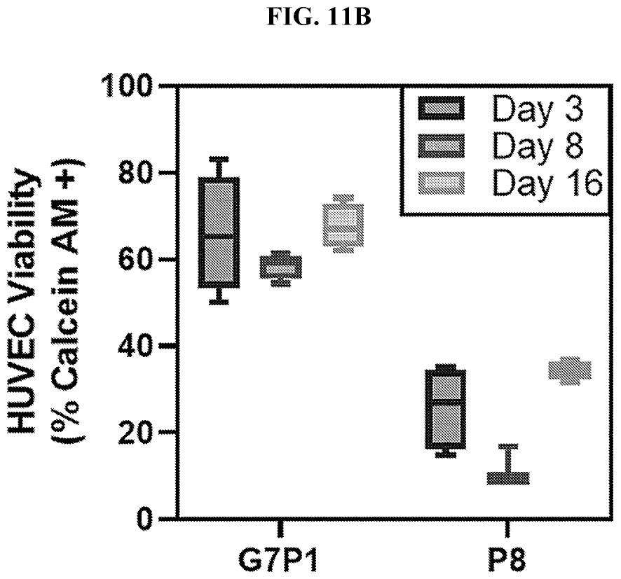

Populations of Human Umbilical Vein Endothelial Cells (HUVECs) were encapsulated in two separate hydrogel formulations: (i) G7(40)P1 [7% GelMA (160P40), 1% PEGDA (35 kDa)]; and (ii) P8(35 kDa) [8% PEGDA, 35 kDa]. After 16 days, hydrogel samples were stained with Calcein AM and ethidium homidmer-1 and imaged for cell aggregation and viability. Results are shown in FIG. 11A. As seen in FIG. 11A, hydrogels which included a combination of chemically modified gelatin (e.g., GelMA) with chemically modified PEG (e.g., PEGDA) reduced cell aggregation of 3D adherent cells (e.g., HUVEC cells) encapsulated within the hydrogel framework.

Both hydrogel formulations were also analyzed on Day 3, Day 8, and Day 16 for Calcein AM percentage (Live)/[Calcein AM percentage (Live)+ethidium homodimer-1 (Dead)] to quantify HUVEC cell viability in each hydrogel. Results are shown in FIG. 11B. As seen in FIG. 11B, hydrogels which included a combination of chemically modified gelatin (e.g., GelMA) with chemically modified PEG (e.g., PEGDA) increased the cell viability of 3D adherent cells (e.g., HUVEC cells) encapsulated within the hydrogel framework. The G7(40)P1 hydrogel maintained a mean cell viability from 60-80% for the duration of the 16 days, while P8(35 kDa) provided a mean cell viability from 20-40%.

### Example 6: Transwell Underside Cell Growth Study—HUVEC

Studies were completed to evaluate the ability of various hydrogel formulations to attach and deliver cells to a membrane with challenging gravitational requirements.

Populations of Human Umbilical Vein Endothelial Cells (HUVECs) were added to two hydrogel precursor formulations: (i) G5(80) [5% GelMA, 80% DoM]; and (ii) P5(35 kDa) [5% PEGDA, 35 kDa] as prepared in Example 3 in PBS. Cell concentrations were about 10 million HUVECs per mL (GFP+).

PET transwell inserts (0.4 μm pore size) were then incubated in serum containing endothelial growth media for 15 minutes. The inserts were dried with eye spears and 13.2 μL of each hydrogel precursor solution (containing HUVECs) was added to individual transwell undersides and exposed to 1 min of high intensity white light (Dolan-Jenner high-intensity LED illuminator MI-LED-US-B1 equipped with a dual-arm gooseneck configuration). The transwells were then inverted onto well plates, such that the hydrogels on each transwell underside was submerged in endothelial growth media supplemented with growth factors and serum. Free HUVECs (i.e., non-hydrogel encapsulated cells) were also added to endothelial growth media in the well plates as a control. Cell growth for each formulation was analyzed on after several days (e.g., Day 4 and Day 11) using GFP+ imaging, and using confocal imaging on Day 11.

### a) GFP+ Imaging Results

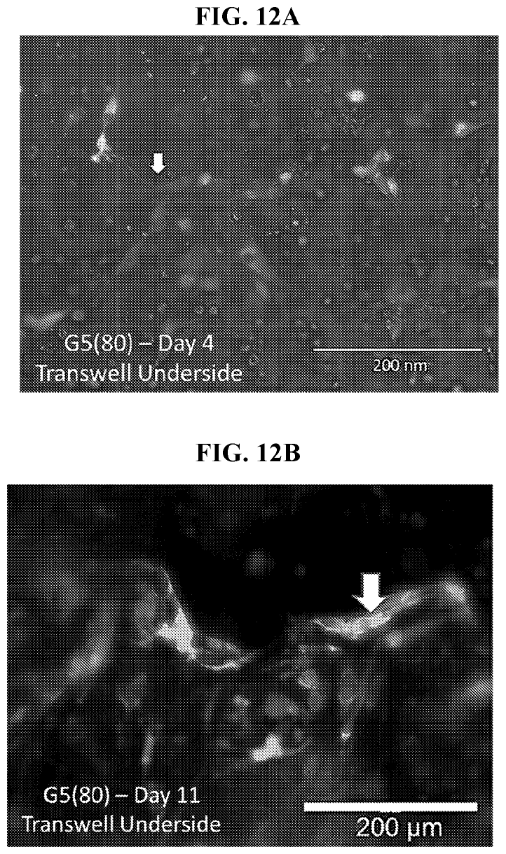

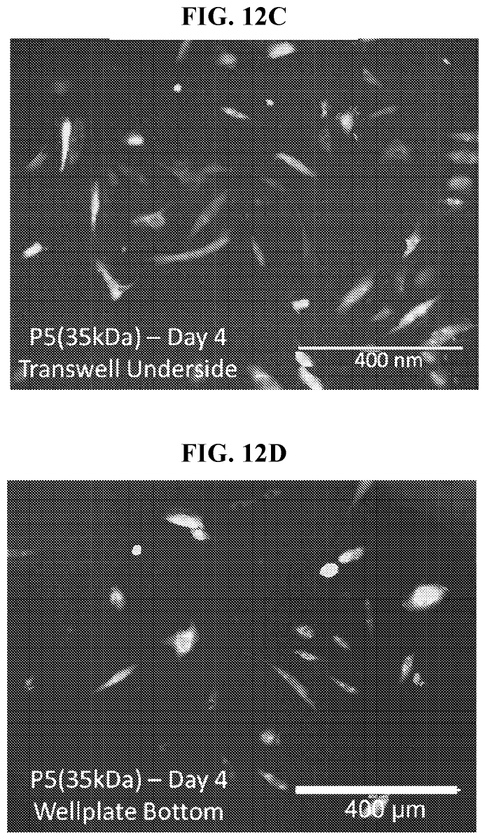

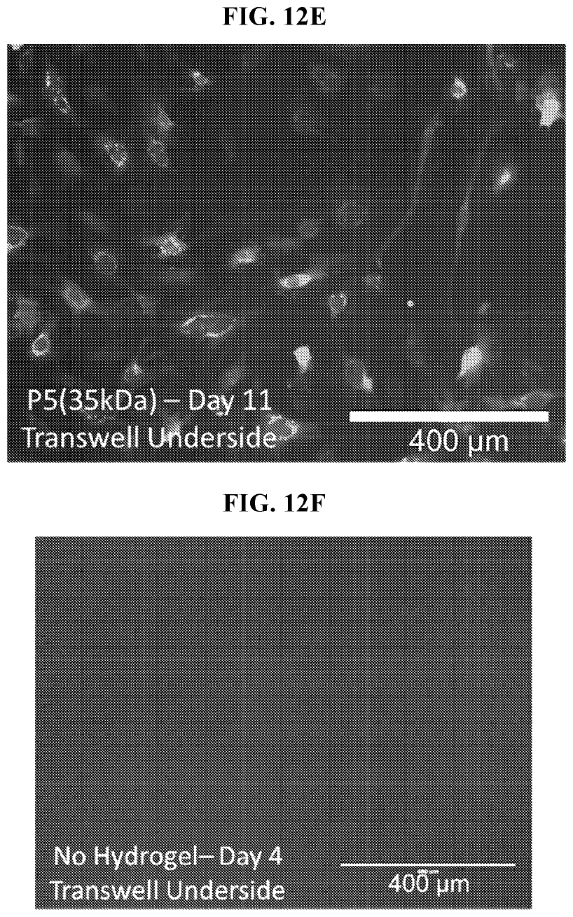

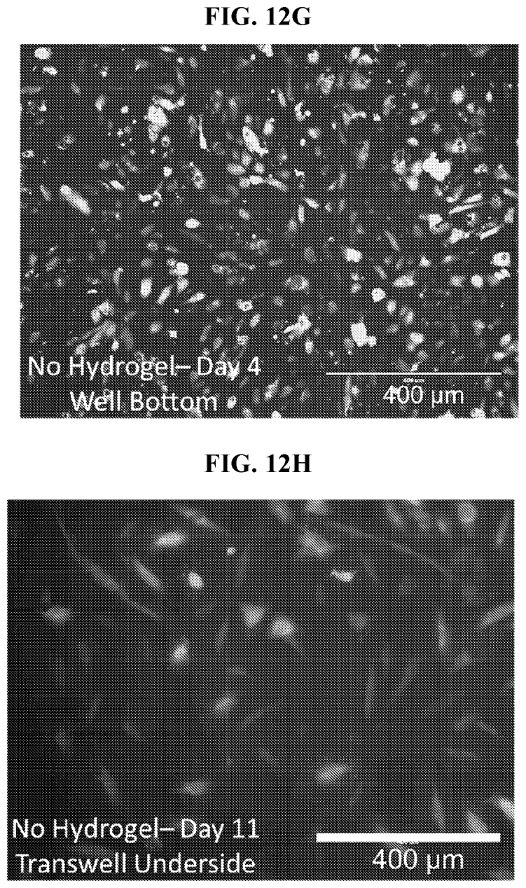

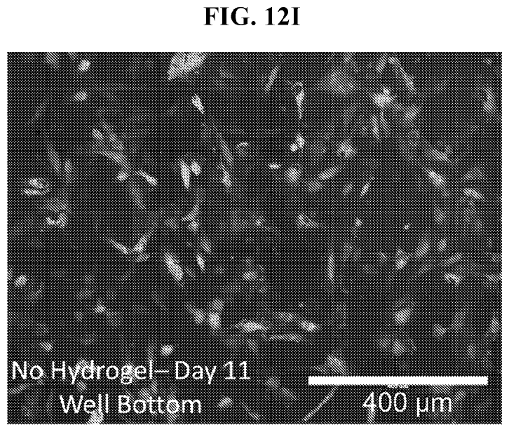

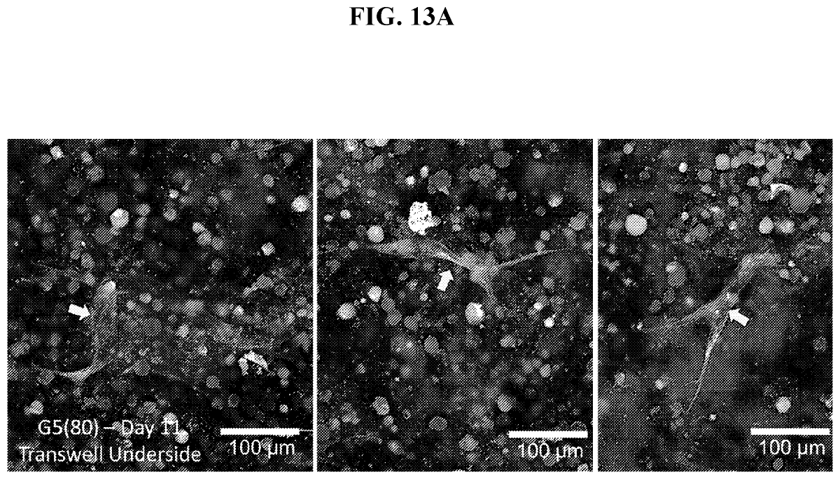

Results for GFP+ Imaging are shown in FIG. 12A-12I.

For G5(80)—After 4 days, samples showed HUVECs remained localized to the underside of the transwell (FIG. 12A), with strong endothelial cell viability and clear cellular network formation (see arrow in FIG. 12A), and only cell debris found localized on the well plate bottom. After 11 days, G5(80) samples continued to show HUVECs localized to the underside of the transwell (FIG. 12B), with continued strong endothelial cell viability and clear cellular network formation (see arrow in FIG. 12B), and only cell debris found localized on the well plate bottom.

For P5(35 kDa)—After 4 days, samples showed partial hydrogel detachment from the transwell underside, with some HUVECs remaining localized to the transwell underside (FIG. 12C), and other HUVECs migrating to the well plate bottom (FIG. 12D). Overall cell density was low in both locations. After 11 days, P5(35 kDa) samples continued to show low cell density and minimal cell network formation on the transwell underside (FIG. 12E), with few remaining viable cells and mostly cell debris found localized on the well plate bottom.

For the free cell control sample (i.e., no hydrogel)—After 4 days, control samples showed minimal HUVEC attachment to transwell underside (FIG. 12F), with most cells migrating to well bottom (FIG. 12G). Overall cell density was low. After II days, control samples continued to show low cell density and minimal cell network formation on the transwell underside (FIG. 12H), with viable cells remaining localized on the well plate bottom (FIG. 12I), with some cell elongation but minimal cell network formation.

### b) Confocal Imaging Results

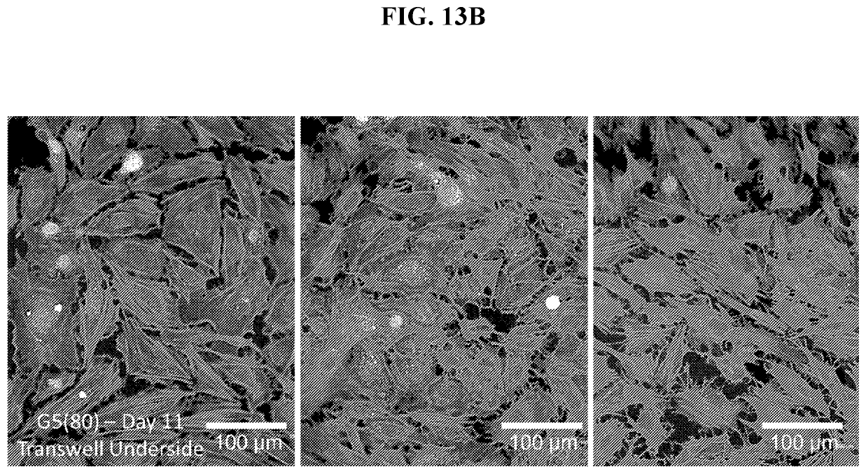

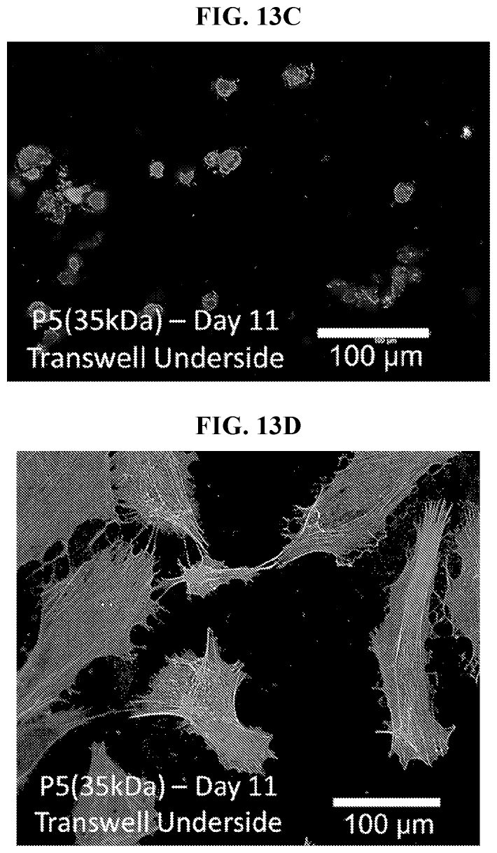

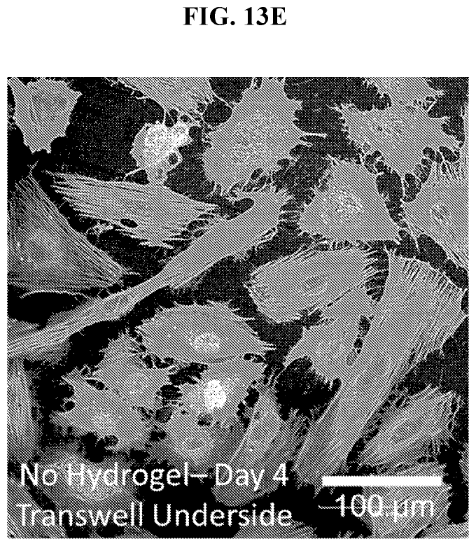

On Day 11, hydrogel samples for G5(80) and P8(35 kDa) from Example 6(a) were stained for the following: Actin (cytoskeleton showing cell spreading and network formation); PECAM/CD31 (endothelial marker); and DAPI (nuclei). Stained samples were then subjected to confocal imaging; results are shown in FIG. 13A-13E.

G5(80) samples showed high HUVEC deposition on the underside of the transwell (FIG. 13A and FIG. 13B), including formation of a dense cellular network with good cell distribution and no evidence of cell clumping.

P8(35 kDa) samples showed limited HUVEC deposition on the underside of the transwell (FIG. 13C and FIG. 13D), including lower cell density with patchy distribution, higher cell debris content, limited cellular spread and network formation, no CD 31 expression (likely related to poor endothelial cell function), and localization of actin filaments to the cellular surface.

Free cell (i.e., no hydrogel) control samples showed lower cell density and poor network formation (FIG. 13E) when compared to G5(80) samples.

### c) Additional Formulation Testing

Additional hydrogel formulations were analyzed according to the procedures of Example 6 (a). Results for transwell underside imaging and analysis are shown in Table 3.

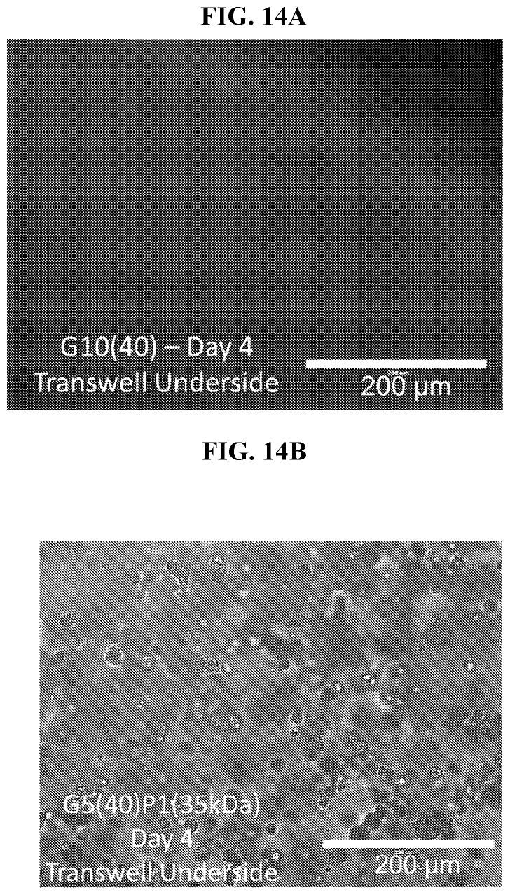

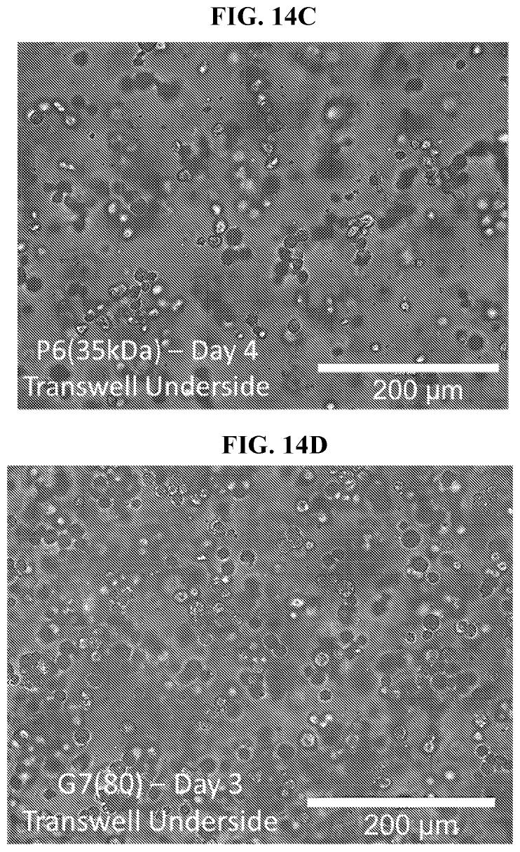

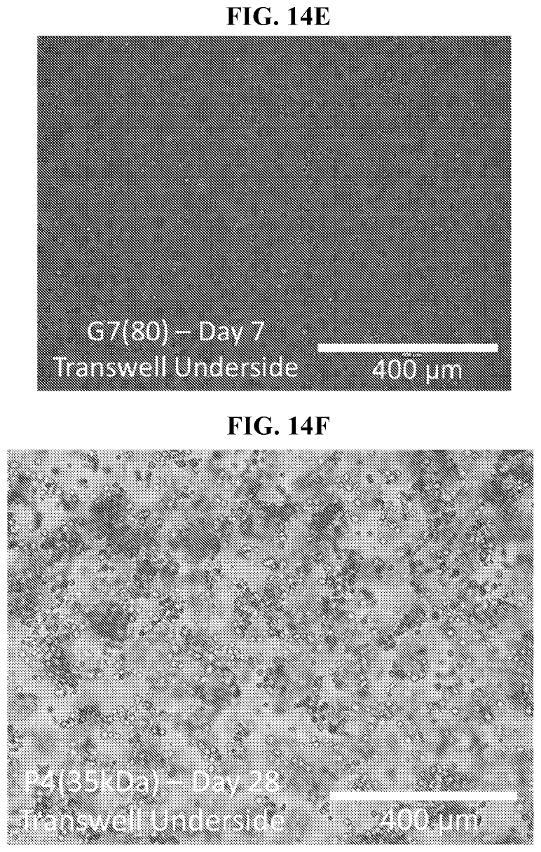

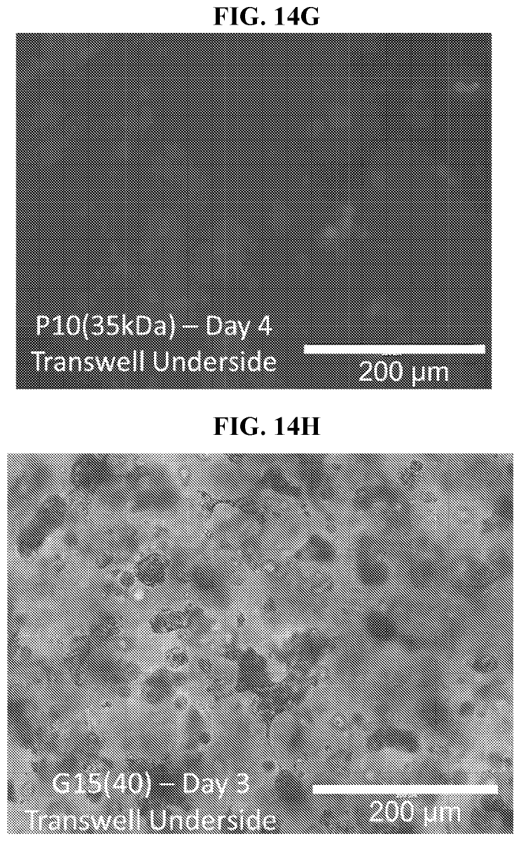

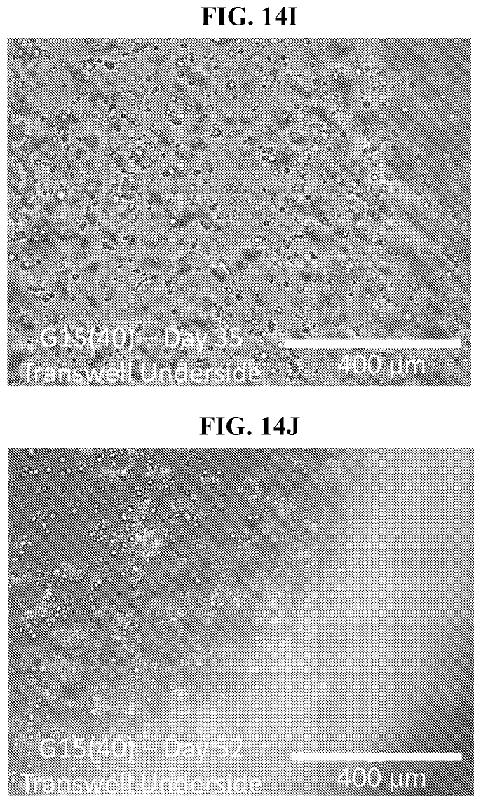

TABLE 3
GFP + HUVEC Analysis

| Formulation | Days | FIG. | Results |
|---|---|---|---|
| G10(40) | 4 | FIG. 14A | Low viability; no spreading or network formation |
| G5(40)P1(35 kDa) | 4 | FIG. 14B | Good viability; no cell spreading; no network |
| | | | formation; some cell aggregation |
| P6(35 kDa) | 4 | FIG. 14C | Good viability; no cell spreading; no network |
| | | | formation; some cell aggregation |
| G7(80) | 3 | FIG. 14D | Low viability; no spreading or network formation; |
| | | | no aggregation |
| G7(80) | 7 | FIG. 14E | After 3 days, VEGF was added to the media. |
| | | | Low viability; no spreading or network formation; |
| | | | no persisting aggregation |
| P4(35 kDa) | 28 | FIG. 14F | Very low viability; no spreading or network |
| | | | formation; increased aggregation |
| P10(35 kDa) | 4 | FIG. 14G | Low viability; no spreading or network formation; |
| | | | some aggregation |
| G15(40) | 3 | FIG. 14H | Good viability; minimal cell spreading; no network |
| | | | formation |
| G15(40) | 35 | FIG. 14I | Good viability; minimal cell spreading; no network |
| | | | formation; hydrogel degradation becomes evident |
| G15(40) | 52 | FIG. 14J | Good viability; minimal cell spreading; no network |
| | | | formation; hydrogel degradation is prominent in the |
| | | | center of the hydrogel |

### d) Study Observations

Study results showed that HUVECs spread more effectively, created stronger cellular networks, and aggregated less within GelMA-based formulations, as compared to PEGDA-based formulations. Study results also showed that higher crosslinking densities (e.g., higher polymer concentration, higher DOM) generally did not promote for strong cell spreading and network formation. GelMA-based materials also provided stronger adhesion to the test surface (i.e., transwell undersurface) and deposit cells more effectively to form a dense cell network, even in challenging environments and growth conditions (e.g., against gravity).

### Example 7: Transwell Underside Cell Growth Study—HRPECs

Studies were completed to evaluate the ability of various hydrogel formulations to attach and deliver cells to a membrane in challenged gravity environments mimicking subretinal space.

Populations of Human Retinal Pigment Epithelium Cells (HRPEC) were added to the following hydrogel precursor formulations: (i) G2(80)H2(500 kDa, 30) [2% GelMA, 80% DOM; 2% HAMA, 500 kDa, ˜30% DOM]; (ii) G5(80) [5% GelMA, 80% DoM]; (iii) G4(80) [5% GelMA, 80% DoM]; (iv) H2(500 kDa, 30) [2% HAMA, 500 kDa, ˜30% DOM]; (v) P5(35 kDa) [5% PEGDA, 35 kDa] as prepared in Example 3 in PBS. Cell concentrations were about 20 million HRPECs per mL.

PET transwell inserts (0.4 μm pore size) were then incubated in serum containing DMEM/F-12 media (supplemented with 10% FBS) for 15 minutes. The inserts were dried with eye spears and 13.2 μL of each hydrogel precursor solution (containing HRPECs) was added to individual transwell undersides and exposed to 1 min of light. The transwells were then inverted onto well plates, such that the hydrogels on each transwell underside was submerged in DMEM/F-12 media (supplemented with 10% FBS). Cell growth for each formulation was imaged and analyzed after several days (e.g., Day 3, Day 8, Day 10, Day 20), which included the use of Calcein AM staining and imaging.

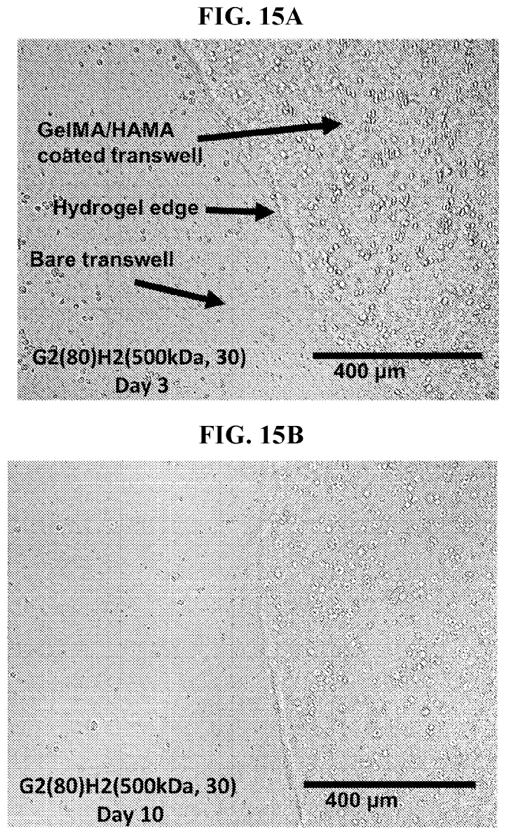

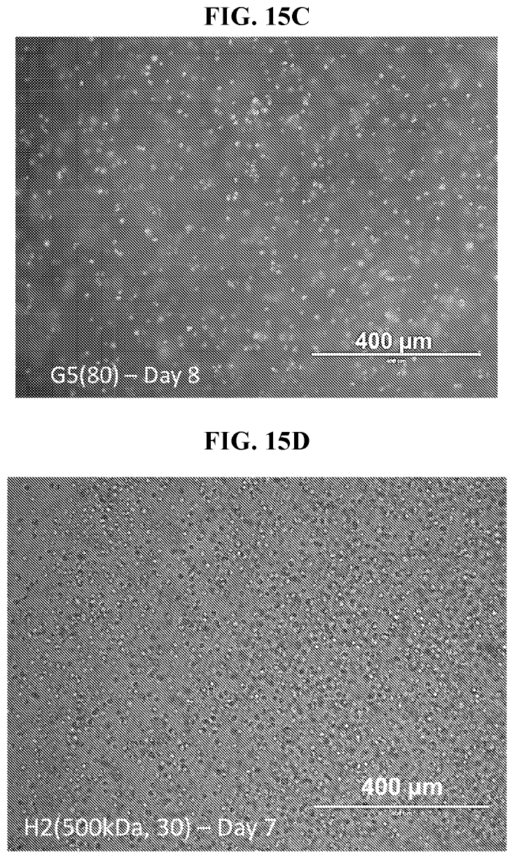

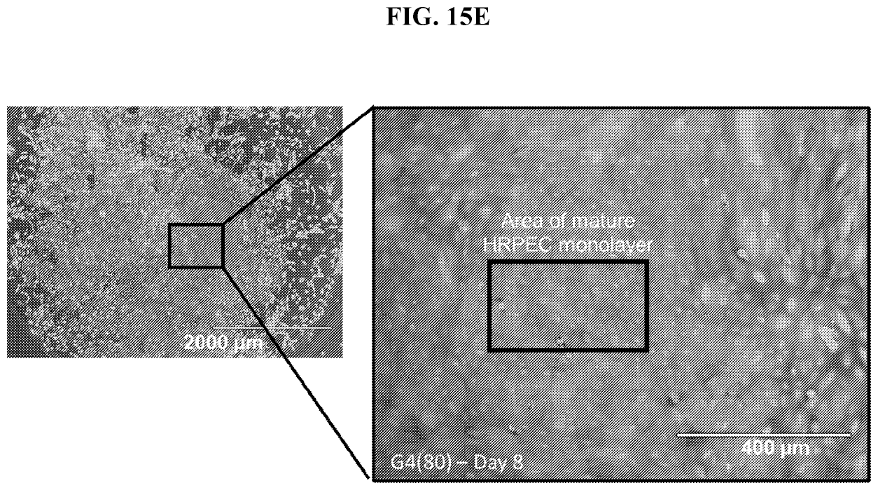

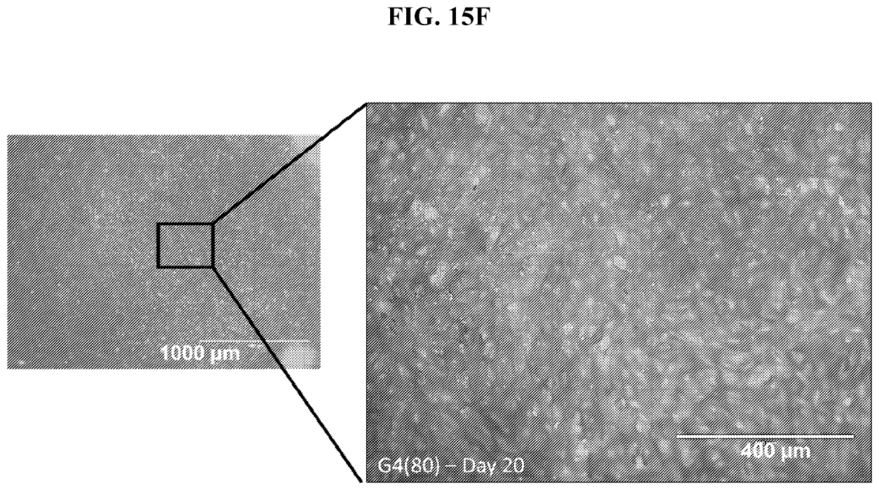

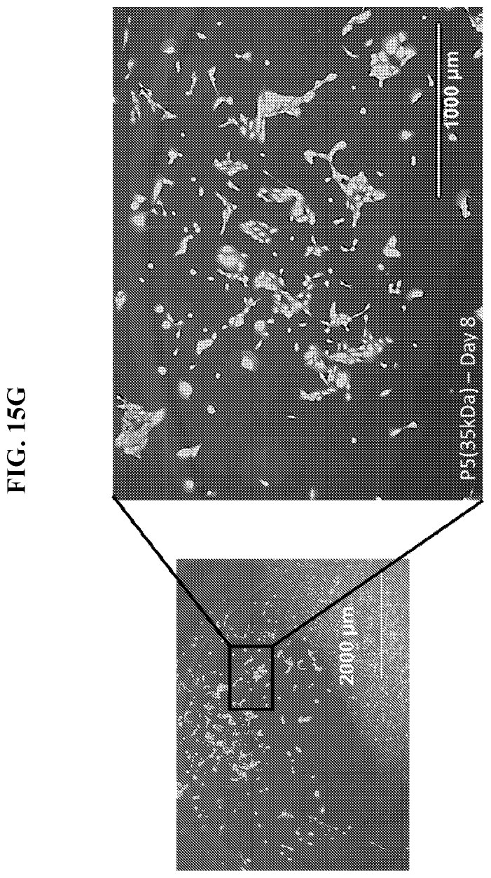

Results are shown in FIG. 15A-15G.

For G2(80)H2(500 kDa, 30)—After 3 days, samples showed HRPECs that remained localized within the hydrogel on to the underside of the transwell, and failed to form a cell monolayer across the surface of the transwell (FIG. 15A). Similar localized results were observed after 10 days, without HRPEC monolayer formation (FIG. 15B).

For G5(80)—After 8 days, samples showed well dispersed rounded cells, but also showed minimal cell spreading and no cell network formation (FIG. 15C).

For H2(500 kDa, 30)—After 7 days, samples showed well dispersed rounded cells, but also showed minimal cell spreading and no cell network formation (FIG. 15D).

For G4(80)—After 8 days, hydrogels were shown to have degraded/detached quickly, but were also shown to have provided a HRPEC monolayer with good coverage and viability, including zones of mature monolayer formation (FIG. 15E). After 20 days, mature HRPEC monolayer was shown to have formed over the entire transwell membrane surface (FIG. 15F).

For P5(35 kDa)—After 8 days, hydrogels were shown to have degraded/detached quickly, and were also shown to have provided a HRPEC monolayer with incomplete coverage and inconsistent monolayer formation (FIG. 15G).

### a) Study Observations

Study results showed that HRPECs have minimal spread and networking within hydrogel formulations that have a high degree of methacrylation, a high molecular weight, and/or a high polymer concentration of hydrogels (e.g., G2(80)H2(500 kDa, 30). HRPECs had improved spread and networking within hydrogel formulations that had a lower degree of methacrylation and lower polymer concentration of hydrogels, thus allowing for effective HRPEC deposition and monolayer formation.

Also, PEG-based hydrogels such as PEGDA were generally shown to have poor bioadhesion and poor biodegradability (often being detached from underneath transwells in as little as 24-48 hours). Remaining HRPECs on the transwell surface were believed to be those that were trapped on the surface of the PEGDA hydrogel during the photopolymerization process. Cells were not able to attach or migrate through the PEGDA polymeric network, remaining trapped and only able to attach to neighboring cells.

### Example 8: Transwell Topside Cell Growth Study—HRPECs

Studies were completed to evaluate the ability of various hydrogel formulations to degrade and deliver cells to a membrane.

Populations of Human Retinal Pigment Epithelium Cells (HRPEC) were added to two hydrogel precursor formulations: (i) G5(10) [5% GelMA, 10% DoM]; and (ii) i) G5(80) [5% GelMA, 80% DoM] as prepared in Example 3 in PBS. Cell concentrations were about 20 million HRPECs per mL.

PET transwell inserts (0.4 μm pore size) were then incubated in serum containing DMEM/F-12 media (supplemented with 10% FBS) for 15 minutes. The inserts were dried with eye spears and about 10 μL of each hydrogel precursor solution (containing HRPECs) was added to individual transwell topsides and exposed to 1 min of light. The transwells were then submerged in DMEM/F-12 media (supplemented with 10% FBS). Free HRPECs (i.e., no hydrogel) were also added to media in the well plates as a control.

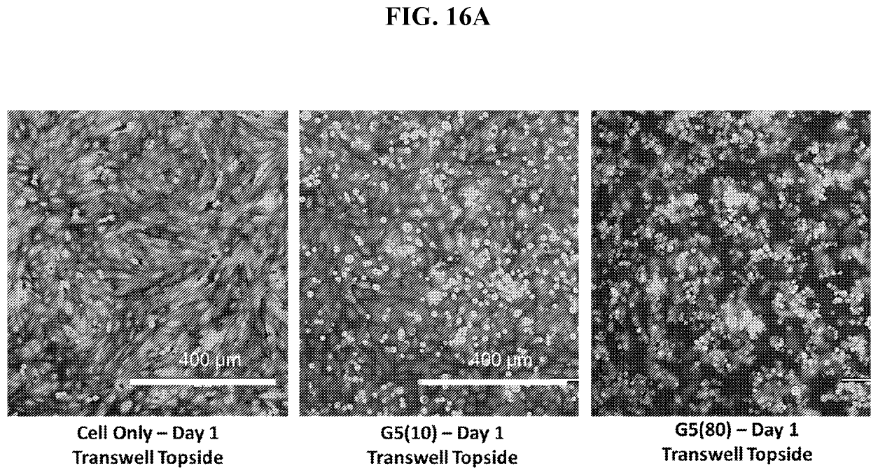

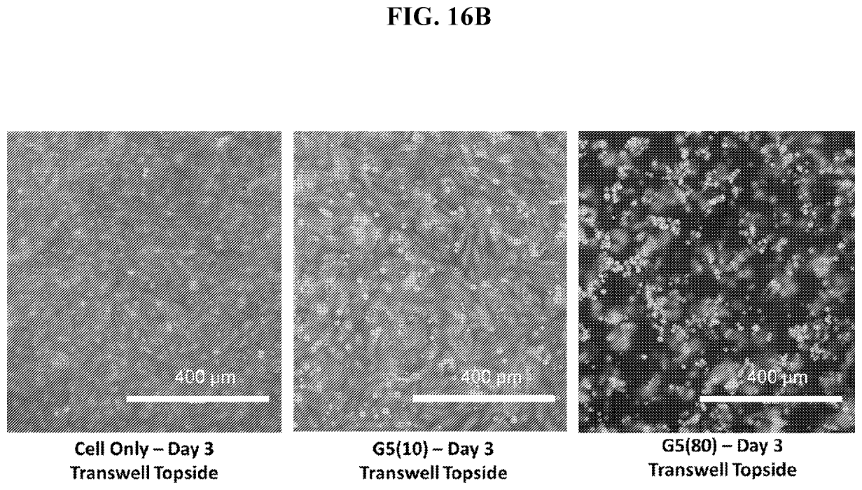

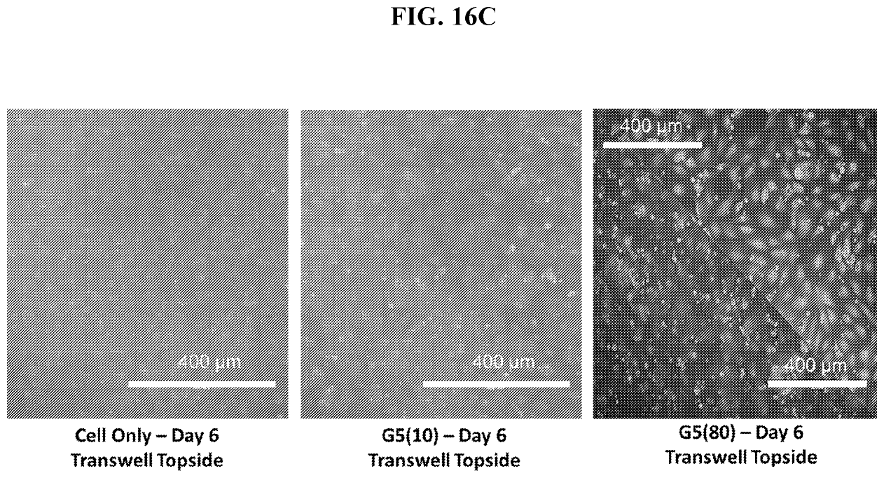

After 24 hours, hydrogel samples were stained with Calcein AM and imaged for cell aggregation and viability (FIG. 16A). Samples were likewise analyzed after 3 days (FIG. 16B), and after 6 days (FIG. 16C).

At 24 hours (FIG. 16A)—Cell-only control samples showed uninhibited cell growth and monolayer formation. G5(10) samples showed hydrogels beginning to degrade, depositing HRPECs, and beginning to form a monolayer networks. G5(80) samples showed no hydrogel degradation and no monolayer formation, with cells generally remaining rounded and suspended within the hydrogel.

At 3 days (FIG. 16B)—Cell-only control samples continued to show uninhibited cell growth and monolayer formation, with cells showing dense packing. G5(10) samples showed hydrogels mostly degraded, with deposited HRPECs forming a clear monolayer network similar to the control sample at 24 hours, and with few rounded, encapsulated cells remaining over the monolayer. G5(80) samples continued to show little hydrogel degradation and little monolayer formation, with cells generally remaining rounded and suspended within the hydrogel.

At 6 days (FIG. 16C)—Cell-only control samples continued to show uninhibited cell growth and monolayer formation, with packed cell density and networking. G5(10) samples showed degraded hydrogels, with a deposited HRPECs monolayer network similar in density and morphology to the control sample, and with few rounded cells remaining over the monolayer. G5(80) samples showed hydrogels beginning to degrade and early monolayer formation in certain regions (top right of FIG. 16C image for G5(80)), while other regions remained undegraded with no monolayer formation (lower left of FIG. 16C image for G5(80)), and with HRPECs remaining rounded and suspended within the hydrogel.

### Example 9: In Vitro Retinal Cell Growth Studies

Studies were completed to evaluate a G5(10) [5% GelMA, 10% DoM]hydrogel formulation for facilitating in vitro delivery and cell growth of stem-cell derived retinal cells, including retinal pigment epithelial (RPE) cells and rod photoreceptor cells.

### A) Monolayer Formation Study

A G5(10) [5% GelMA, 10% DoM]hydrogel precursor formulation was prepared according to the general procedures in Example 3, with reduced concentrations of photopolymerization initiator mixture. A population of Human Retinal Pigment Epithelium Cells (HRPEC), derived from induced pluripotent stem cells (iPSC), was then added to the G5(10) hydrogel precursor formulation (˜20 million cells/mL). iPSC-derived RPEs were also added to a saline solution as a control. About 200 μL of each formulation was injected (in PBS) through a MedOne Subretinal PolyTip® 38G (outer diameter) (41G inner diameter) subretinal cannula (with a standard 1 mL luer-lok plastic syringe) at a controlled rate of 200-300 μL/min (flow rate controlled with a syringe pump) onto 6-well transwell plates with 0.4 μm polyester (polyethylene terephthalate) membranes. The polyester membranes were included to mimic native Bruch's membrane; membrane is permeable to nutrients and proteins, but not to cells. The injected formulations were then photo crosslinked in 10 μL drops using white light.

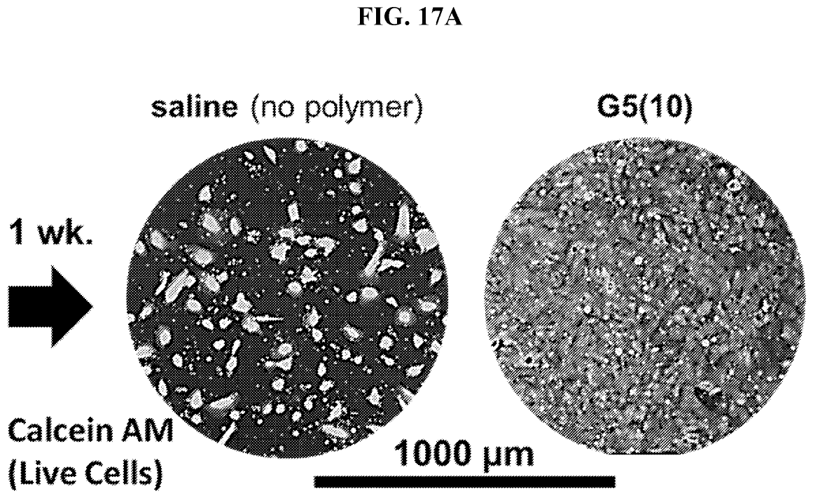

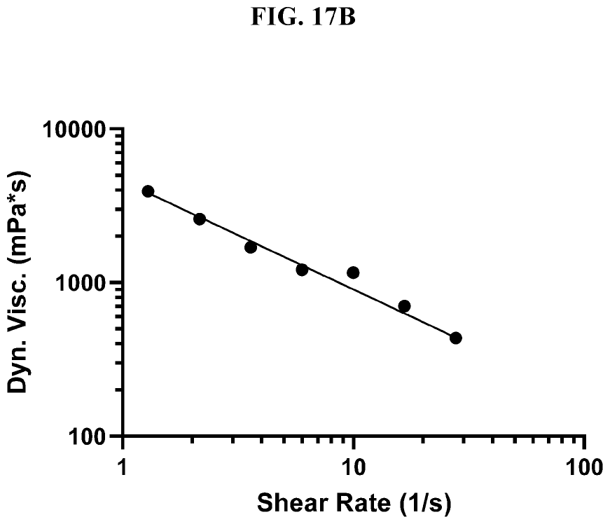

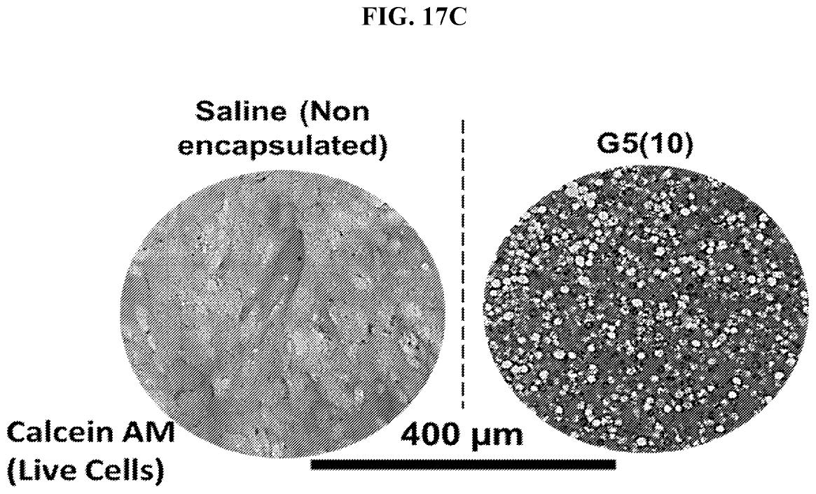

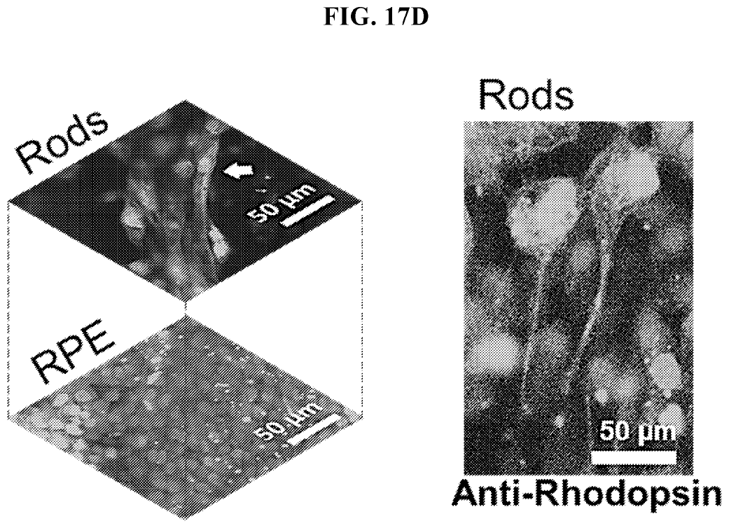

After one week, live-cell imaging was completed using Calcein AM. Results are shown in FIG. 17A. Study results showed that RPE cells were able to slowly degrade the G5(10) hydrogel substrate after crosslinking and then migrate to the transwell membrane. The RPE cells had formed a clear cellular monolayer after one week, mimicking target anatomic patterning of functional RPE cells (See FIG. 17A). The RPE cells delivered in the saline (control) formulation failed to form observable cellular monolayers after one week, despite being delivered at the same seeding density as the cells in the hydrogel formulation.

Fluid shear rates related to extrusion through the 38G (41G inner) small-diameter needle were measured during the sampled delivery. Results (see FIG. 17B) showed that GelMA pre-polymers can be shear thinning in precursor polymer solutions, thus enabling protective plug-flow dynamics through small diameter needles that protects the passenger cells.

B) iPSC De-Differentiation Study

Precursor formulations from Example 9a were prepared at ˜1 million cells/mL, and then injected (in PBS) onto 6-well transwell plates with 0.4 μm polyester (polyethylene terephthalate) membranes using the same delivery system and conditions as Example 9A. The injected formulations were then photo crosslinked in 10 μL drops using white light. After one week, live-cell imaging was completed using Calcein AM. Results are shown in FIG. 17C. Study results showed that RPE cells had formed a monolayer, with cells retaining a cuboidal morphology and pigmentation patterns that are characteristics of standard, healthy RPE morphology. The RPE cells delivered in the saline (control) formulation became elongated and highly proliferative, and failed to retain their cuboidal morphology and RPE pigmentation patterns indicating de-differentiation.

Without being bound by theory, study observations show that the hydrogel substrate provides a localized and condensed environment for the RPE cells, thus enabling monolayer formation with the characteristic RPE morphology by reducing RPE de-differentiation due to environmental factors.

### C) Rod Photoreceptor Delivery Study

A G5(10) [5% GelMA, 10% DoM]hydrogel precursor formulation was prepared according to the general procedures in Example 3, with reduced concentrations of photopolymerization initiator mixture. A population of rod photoreceptor cells, derived from human retinal organoids (Lako Lab, Newcastle University), was then added to the G5(10) hydrogel precursor formulation. The formulations were then injected (in PBS) onto RPE monolayers in 6-well transwell plates with 0.4 μm polyester (polyethylene terephthalate) membranes, using the same delivery system and conditions as Example 9A. The injected formulations were then photo crosslinked using white light.

After two weeks, live-cell imaging was completed using anti-CD73 and anti-rhodopsin imaging. Results are shown in FIG. 17D. Study results showed that rod photoreceptor cells (left, top of FIG. 17D) were localized over the RPE monolayer (left, bottom of FIG. 17D) and remained viable at two weeks, with both the rod photoreceptor cells and RPE cells expressing CD73. Only the rod photoreceptor cells were shown to express rhodopsin (right of FIG. 17D).

### Example 10: In Vivo Retinal Cell Growth Studies

Studies were completed to evaluate cell localization and viability of retinal pigment epithelial cells (RPE) after a single subretinal (SR) injection in swine in one of three G5 [5% GelMA]hydrogel formulations polymerized in situ via intraocular procedure light.

Four test groups included 200 μl injections into 6 eyes (for each group, 24 eyes and 12 animals total):

Group 1 (Control): Saline+200 k RPE cells; Group 2: G5(10) [5% GelMA, 10% DoM]+200 k RPE cells; Group 3: G5(80) [5% GelMA, 80% DoM]+200 k RPE cells; Group 4: [5% GelMA, 80% DoM]G5(80)+1 m RPE cells.

Animals in Groups 2-4 received a single SR injection of RPE with the designated hydrogel formulation. Group 1 was administered a single injection of RPE in saline as control. Prednisone oral was given pre-op and throughout the study. For the injection, 38G/41G MedOne subretinal needle equipped with 6-7 in extension tubing was used. Hydrogel precursors and cell mixtures were prepared fresh prior to each group. Constellation 23G light pipe was used for radiation, which at max power emitted approximately 27 mW of light power (as measured at 517 nm).

All animals had ophthalmic examinations, color fundoscopy, and ocular coherence tomography (OCT) post-injection and on Days 1, Day 3, 7, 14, 21, 28 post-injection. All enrolled eyes were stained for STEM121 (transplanted human cells), Iba-1 (microglia/white blood cells), RPE65 (RPE cells), and DAPI (all nuclei).

Two eyes from each group were also selected for immunofluorescent staining. Blocks were sectioned sagittally (14 μm) at 5 levels across the eye: nasal, peripheral, midway between nasal and optic nerve head (ONH), ONH, midway between the ONH and temporal, and temporal peripheral. At least 10 slides/level were collected. Slides were scanned during acquisition for the cell depot. A series of 8-10 slides/eye was selected for IHC.

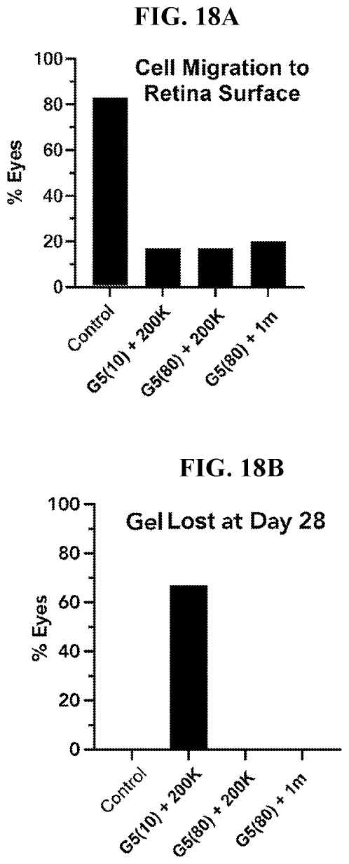

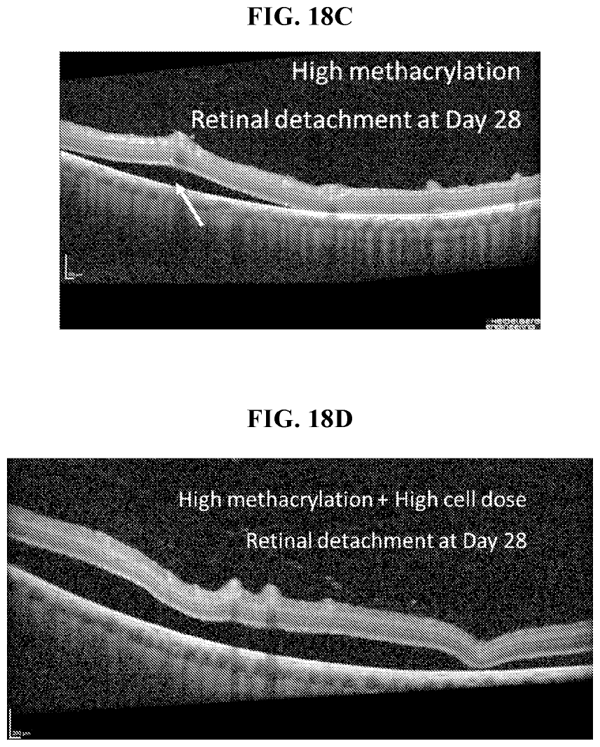

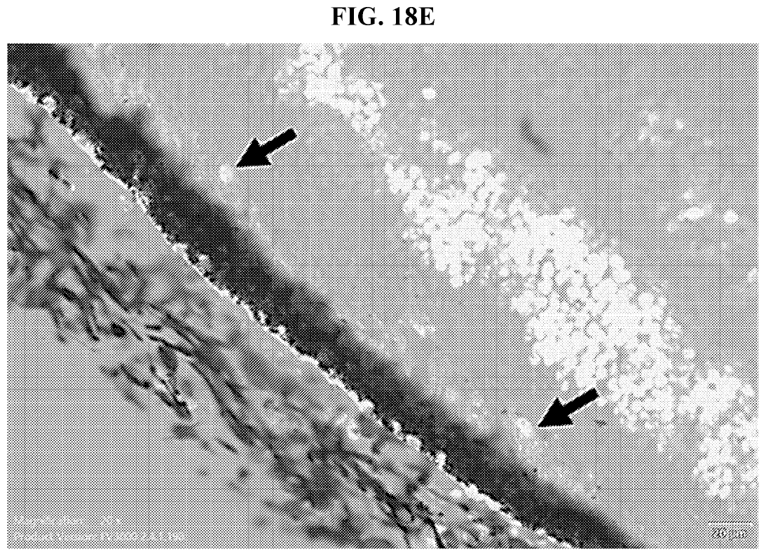

Results of observations related to cell migration to retina surface are shown in FIG. 18A. The saline control formulations exhibited more cell migration (above 80%) and corresponding epiretinal adverse events due to poor cell localization (i.e., cells outside of subretinal area). This finding was confirmed by histological examination of the eye after day 28, in which vitreous cells were found, and subretinal hydrogel formulations exhibited minimal cell migrations of about 20% or below.

Results of observations related to hydrogel degradation after 28 days are shown in FIG. 18B. The Group 1 (control) did not include hydrogel. For Group 2, only about 33% of hydrogel remained after 28 says (i.e., about 67% lost). Group 3 and Group 4 had about 100% hydrogel remaining after 28 days (i.e., about 0% lost). After 28 days, hydrogels with high 80% methacrylation (Group 3 and Group 4) showed retinal detachment from underlying layers, as seen in FIG. 18C and FIG. 18D. Staining results did show successful human stem cell transplant onto native pig RPE layers, which were not disrupted (see FIG. 18E, wherein the arrows represent human cells).

### Example 11: Radical Propagator Studies

Studies were completed to evaluate N-vinylcaprolactam (NVC), N-Vinylpyrrolidone (NVP), and Ethylene Glycol Diacrylate (EGDA) in hydrogel formation.

Each of the three propagators (NVC, NVP, EGDA) were combined with G5(80) [5% GelMA, 80% DoM] at 10 uL/mL propagator concentration, and then gelled. Average Young's Modulus (kPa) for resulting hydrogels were as follows: EGDA [7.2494 kPa]; NVP [6.4995 kPa]; NVC [3.4744 kPa]. NVP and EGDA were also tested for a minimum concentration in G5 (10) and G5(80) which would still allow for hydrogel formation (1 minute crosslinking at ˜0.43 W). Minimum propagator concentrations for hydrogel formation were as follows: G5 (10) [NVP—5 uL/mL; EGDA—1.5 uL/mL]; G5 (80) [NVP—0.3 uL/mL; EGDA—0.1 uL/mL]

G5(10)—1.5 uL/mL EGDA and G5(10)—5 uL/mL NVP were then studied for RPE cell encapsulation (with PBS as a control) through needle extrusion. After initial extrusion, all three samples showed cells that retained pigmentation and morphology. After 24 hours, PBS cells lose pigmentation and show early signs of epithelial-to-mesenchymal transition (EMT). Both hydrogel formulations showed cells that retained pigmentation and morphology. The same results were observed at Day 6.

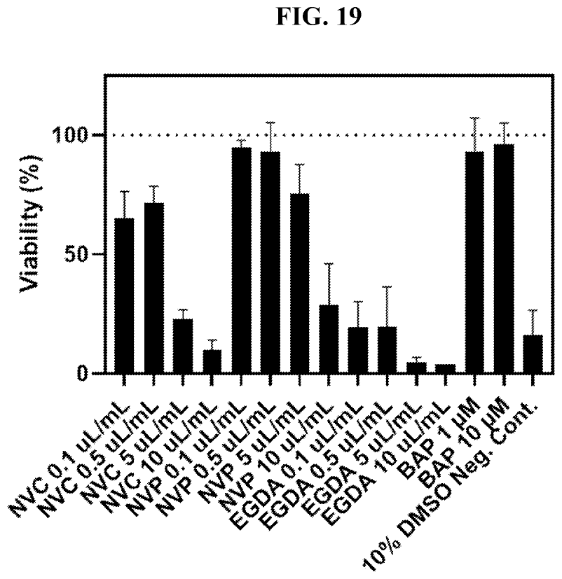

Cytotoxicity studies were also completed with RPE cells for: NVC, NVP, EGDA, and phenylacetyl bromide (BAP), with 10% DMSO as a control. Cell viability results after 18 hours of cell incubation in each of the four propagators is shown in FIG. 19. Results showed that NVP and BAP had higher average cell viability than NVC at all relative concentrations, and the EGDA had low cell viability results.

### Example 12: G5 Formulation Studies with N-Vinylpyrrolidone (NVP)

G5 formulations [5% GelMA, varying DoM and MW] with different average molecular weights was studied for gelation and cell encapsulation properties. Specifically, G5(10) 160 kDa, G5(60) 90 kDa, and G5(45) 160 kDa were mixed with standard photoinitiator concentration (but with 1% NVP instead of NVC) and 5 M ARPE-19 cells/mL of formulation (˜200 k cells total per gel). Samples (including extruded saline with cells) were injected into sample containers using a MedOne 38/41G Subretinal Needle (300 L/min injection rate), and a light pipe (set to 26-27 mW) was used to mimic Constellation vitrectomy set-up (30 second exposure). Saline (non-extruded) was used as a normalizing control.

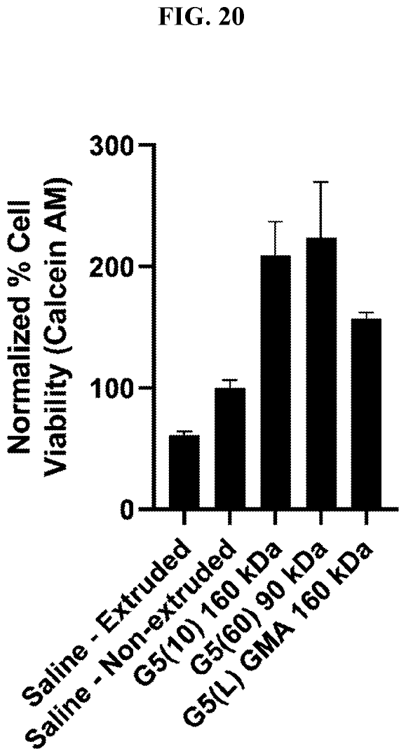

Cell viability results using Calcein AM (normalized to non-extruded saline) are shown in FIG. 20. All formulations showed normalized cell viability of at least 150%, with G5(10) 160 kDa and G5(60) 90 kDa showing over 200% normalized cell viability. After 48 hours, gel degradation was evident for all three hydrogel samples, with cells attached to the transwell surface and early monolayer formation. Few cells remain rounded and unattached.

Further studies were completed to analyze gelation properties of various G4 formulations [4% GelMA, varying DoM and MW] and G5 formulations [5% GelMA, varying DoM and MW] using NVP as a radical propagator. Table 4 presents study conditions and results for the various formulations.

TABLE 4
NVP Gelation Studies

| | | Autoclaved | | Optical | | |
|---|---|---|---|---|---|---|
| | Approx. | (30 min at | | Power | Gelation | Hydrolytic |
| Formulation | MW (kDa) | 121° C.) | Propagator | (mW) | (after 2 min) | Degradation |
| G5(40) | 160 | Yes | 0.1% NVP | 26 | No | |
| G5(40) | 160 | Yes | 1% NVP | 26 | Soft gel | 2 weeks |
| G5(40) | 160 | No | 0.1% NVP | 26 | No | |
| | | | | 100 | Soft gel | |
| G5(40) | 160 | No | 1% NVP | 26 | Yes | 2 weeks |
| G5(10) | 160 | No | 1% NVP | 26 | No | |
| G5(60) | 90 | No | 1% NVP | 26 | No | |
| G5(60) | 90 | No | 1% NVP | 26 | (After 30 | 5 days |
| | | | | | seconds) - | |
| | | | | | Soft, elastic, | |
| | | | | | viscous | |

Additional polymer blends using GelMA concentrations from 0.5% to 3.0% were also tested for gelation and cell encapsulation properties. Table 5 presents study conditions and results for the various formulations.

TABLE 5
NVP Gelation Studies - Low GelMA %

| | | Optical | Gelation | Hydrolytic |
|---|---|---|---|---|
| Formulation | Propagator | Power (mW) | (after 1 min) | Degradation |
| G3(10)G0.5(60) | 1% NVP | 26 mW | No | |
| G3(10)G1(60) | | | | |
| G3.5(10)G1(60) | | | | |
| G3.5(10)G1.5(60) | | | | |
| G3(10)G2(60) | | | | |
| G2(10)G1.5(60) | | | | |
| G1.5(10)G2(60) | | | | |
| G1(10)G2.5(60) | | | Soft Gel | Less than 3 days |

### Example 13: Combination Formulation Study

A G1(160/10)G2.5(90/60) blend was studied with various photoinitiator formulations (including triethanolamine (TEOA) at 1.5% v/v) and polymerization conditions. Table 6 presents study conditions and results for the various formulations.

TABLE 6
Combination Formulation Study

| | | | | Gel Time | | |
|---|---|---|---|---|---|---|
| Reaction | | EYDS | NVP | (at 26 | | |
| Conditions | Formulation | Concentration | Concentration | mW) | | Gel Properties |
| Rxn 1 | G1(160/10)G2.5(90/60) | 10 μM | 3.5 μM/mL | 2 | minutes | Elastic, viscous |
| | | | | 4 | minutes | Soft hydrogel |
| Rxn 2 | | 50 μM | 3.5 μM/mL | 2 | minutes | Elastic, viscous |
| | | | | 4 | minutes | Soft hydrogel |
| Rxn 3 | | 50 μM | 5 μM/mL | 1 | minute | Elastic, viscous |
| | | | | 2 | minutes | Soft hydrogel |
| Rxn 4 | | 50 μM | 5 μM/mL + | 30 | seconds | Highly viscous |
| | | | 0.5% 2K | 40 | seconds | Soft hydrogel |
| | | | PEGDA | | | |

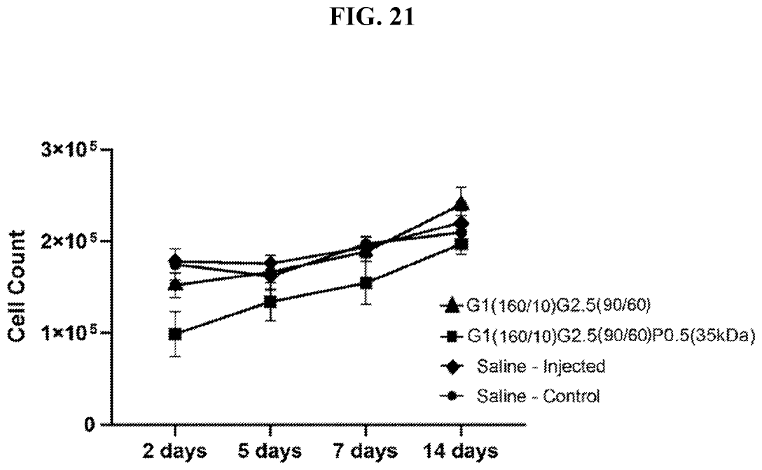

Rxn 3 and Rxn 4 were further studied. 10 M cells/mL were added to each of Rxn 3, Rxn4, and PBS (as control). The Rxn 3 sample, Rxn 4 sample, and a portion of the PBS control were extruded onto a transwell surface, as well as unextruded PBS. After two weeks, both Rxn 3 and Rxn 4 showed healthy RPE monolayer growth, similar to the PBS controls, with the Rxn 3 sample providing a high cell count than either PBS control sample. Cell count results are show in FIG. 21.

We claim:

1. A polymer composition, comprising:
(i) between about 0.5% to about 5.0% w/v of chemically modified gelatin;
(ii) at least one polymer crosslinking initiator; and
(iii) at least one cell.

2. The polymer composition of claim 1, wherein the polymer crosslinking initiator comprises one or more light-activated photo-initiators; optionally one or more photo-initiators activated by visible light.

3. The polymer composition of claim 2, wherein the polymer crosslinking initiator comprises: (i) eosin Y, N-vinylcaprolactam (NVC), triethanolamine, or any combination thereof; (ii) eosin Y disodium salt (EYDS), N-vinylcaprolactam (NVC), triethanolamine, or any combination thereof; or (iii) eosin Y disodium salt (EYDS), N-Vinylpyrrolidone (NVP), triethanolamine, or any combination thereof.

4. The polymer composition of claim 2, wherein the polymer crosslinking initiator comprises: (i) about 50 μM eosin Y or eosin Y disodium salt (EYDS); (ii) from about 3.5 to about 5.0 μL/mL of N-vinylcaprolactam (NVC) or N-Vinylpyrrolidone (NVP); and (iii) triethanolamine.

5. The polymer composition of claim 2, wherein the polymer crosslinking initiator comprises: (i) about 50 μM eosin Y disodium salt (EYDS); (ii) about 5.0 μL/mL N-Vinylpyrrolidone (NVP); and (iii) about 1.5% v/v of triethanolamine.

6. The polymer composition of any one of claims 1-5, wherein the chemically modified gelatin is acrylated gelatin.

7. The polymer composition of claim 6, wherein the acrylated gelatin has a degree of acrylation from about 5-40%; optionally from 5-20%; optionally about 5%, about 10%, or about 15%.

8. The polymer composition of any one of claims 1-5, wherein the chemically modified gelatin is methacrylated gelatin (GelMA).

9. The polymer composition of claim 8, wherein the GelMA has a degree of methacrylation from about 5-40%; optionally from 5-20%; optionally about 5%, about 10%, or about 15%.

10. The polymer composition of any one of claims 1-9, wherein the polymer composition comprises from about 2% to about 5% w/v of the chemically modified gelatin; optionally from about 3% to about 5% w/v of the chemically modified gelatin.

11. The polymer composition of claim 10, wherein the polymer composition comprises from about 3% to about 4% w/v of the chemically modified gelatin; optionally about 3.5% w/v.

12. The polymer composition of claim 10, wherein the polymer composition comprises from about 3% to about 4% w/v GelMA; optionally about 3.5% w/v GelMA.

13. The polymer composition of any one of claims 1-10, wherein the polymer composition comprises a combination of a first GelMA mixture and a second GelMA mixture.

14. The polymer composition of claim 13, wherein the polymer composition comprises from about 0.5% to about 3% w/v of the first GelMA mixture, and about 0.5% to about 3% w/v of the second GelMA mixture; optionally from about 0.5% to about 1.5% w/v of the first GelMA mixture and about 1.5% to about 3% w/v of the second GelMA mixture; optionally about 1% w/v of the first GelMA mixture and about 2.5% w/v of the second GelMA mixture.

15. The polymer composition of claim 13 or claim 14, wherein the first GelMA mixture includes GelMA having a high average molecular weight and a low degree of methacrylation (DOM); and the second GelMA mixture includes GelMA having a low average molecular weight and a high (DOM); optionally wherein the first GelMA mixture includes GelMA having an average molecular weight from 140-180 kDa and a DOM from 5% to 40%, and the second GelMA mixture includes GelMA having an average molecular weight from 75-115 kDa and a DOM from 50% to 80%; optionally wherein the first GelMA mixture includes GelMA having an average molecular weight from 140-180 kDa and a DOM from 5% to 20%; and the second GelMA mixture includes GelMA having an average molecular weight from 80-100 kDa and a DOM from 50% to 70%.

16. The polymer composition of claim 13 or claim 14, wherein the first GelMA mixture includes GelMA having an average molecular weight of about 160 kDa and a DOM of about 10%; and the second GelMA mixture includes GelMA having an average molecular weight of about 90 kDa and a DOM of about 60%.

17. The polymer composition of claim 13, wherein the polymer composition comprises: about 1% w/v of a first GelMA mixture which includes GelMA having an average molecular weight of about 160 kDa and a DOM of about 10%; and about 2.5% w/v of a second GelMA mixture which includes GelMA having an average molecular weight of about 90 kDa and a DOM of about 60%.

18. The polymer composition of any one of claims 1-17, wherein the at least one cell comprises an endothelial cell; optionally a human umbilical vein endothelial cell (HUVEC).

19. The polymer composition of any one of claims 1-17, wherein the at least one cell comprises an epithelial cell; optionally a human retinal pigment epithelium cell (HRPEC); optionally an HRPEC derived from embryonic stem cells or induced pluripotent stem cells (iPSC).

20. The polymer composition of any one of claims 1-17, wherein the at least one cell comprises an ocular cell; optionally an ocular cell derived from a pluripotent stem cell or an embryonic stem cell.

21. The polymer composition of any one of claims 1-20, further comprising at least 0.1% (w/v) of a hydrophilic non-ionic surfactant; optionally wherein the hydrophilic non-ionic surfactant comprises at least one poloxamer surfactant such as Poloxamer 407; optionally where in the composition comprises about 0.2% (w/v) of a poloxamer surfactant such as Poloxamer 407.

22. The polymer composition of any one of claims 1-21, further comprising one or more non-cell therapeutic agents; optionally wherein the one or more non-cell therapeutic agents comprises a small molecule or protein therapy.

23. A precursor polymer composition, comprising the polymer composition of any one of claims 1-22.

24. A gel polymer composition, wherein the gel polymer composition is formed by photocrosslinking a precursor polymer composition of claim 23; optionally wherein the gel polymer composition is a hydrogel.

25. The gel polymer composition of claim 24, wherein the shape of the polymer composition is conformed to the shape of the target surface; optionally wherein the polymer composition is conformed to the convex, concave, or curved shape of the target surface.

26. The gel polymer composition of claim 24, wherein the polymer composition is in the shape of a cylinder; optionally wherein the polymer composition is in the shape of a disk cylinder or a rod cylinder; optionally wherein the polymer composition is in the shape of rod cylinder having a diameter of about 0.75 mm and a length about 3 mm, or having a diameter of about 0.75 mm and a length about 6 mm.

27. A method for treating and/or repairing a defect, injury, and/or disease in a target soft tissue of a subject, said method comprising:
providing a precursor polymer composition of claim 23;
administering the precursor polymer composition onto or under a surface of the target soft tissue of the subject, optionally the location of the soft tissue defect, injury, and/or disease; and
crosslinking the precursor polymer composition by exposing the polymer crosslinking initiator in the polymer composition to crosslinking conditions, wherein the crosslinking of the precursor polymer composition produces a gel polymer composition.

28. A method for treating a defect, injury, and/or disease in a target soft tissue of a subject, said method comprising:
providing a gel polymer composition of any one of claims 24-26; and
administering the gel polymer composition onto, under, or near a surface of the target soft tissue of the subject; optionally at the location of the soft tissue defect, injury, and/or disease.

29. The method of any one of claims 27-28, wherein target soft tissue is ocular tissue; optionally subconjunctival ocular tissue or retinal ocular tissue.

30. The method of claim 29, wherein the polymer composition is applied onto or under the surface of the ocular tissue by subconjunctival injection, subretinal injection, or suprachoroidal injection.

31. The method of any one of claims 27-30, wherein the defect, injury, and/or disease of the target soft tissue comprises an ocular defect, injury and/or disease; optionally an ocular ulcer; optionally a corneal ulcer from infections, injuries, perforations, or other defects.

32. The method of claim 27-30, wherein the ocular defect, injury and/or disease comprises a retinal degeneration disease; optionally age-related macular degeneration (AMD) or retinitis pigmentosa.
